(19) United States
(12) Patent Application Publication
(10) Pub. No.: US 2024/0158448 A1
(43) Pub. Date: May 16, 2024

(54) MULTIVALENT NANO-'SELF' PEPTIDES AND USES THEREOF

(71) Applicant: THE TRUSTEES OF THE UNIVERSITY OF PENNSYLVANIA, Philadelphia, PA (US)

(72) Inventors: Dennis Discher, Philadelphia, PA (US); Abdelaziz Jalil, Philadelphia, PA (US)

(21) Appl. No.: 18/546,695

(22) PCT Filed: Feb. 18, 2022
(86) PCT No.: PCT/US22/17000

§ 371 Date: Aug. 16, 2023

Related U.S. Application Data
(60) Provisional application No. 63/151,194, filed on Feb. 19, 2021.

(51) Int. Cl. C07K 14/00; A61P 37/06

(52) U.S. Cl. CPC C07K 14/001; A61P 37/06; C07K 2319/00

(57) ABSTRACT

The present disclosure includes methods and compositions that can efficiently enhance macrophage-mediated phagocytosis of target cells by inhibiting the SIRPα/CD47 interaction in a way that minimizes off-target toxicity.

### CROSS-REFERENCE TO RELATED APPLICATION

The present application claims priority under 35 U.S.C. § 119(e) to U.S. Provisional Patent Application No. 63/151,194, filed Feb. 19, 2021, which is hereby incorporated by reference in its entirety herein.

### STATEMENT REGARDING FEDERALLY SPONSORED RESEARCH OR DEVELOPMENT

This invention was made with government support under R01-HL124106-06 awarded by the National Institutes of Health and DMR-1120901 awarded by the National Science Foundation. The government has certain rights in the invention.

### BACKGROUND OF THE INVENTION

Clearance of cancer cells, or any other foreign cell, by immune cells requires a balanced response of competing inhibitory and activating signals. Cancer cells exploit anti-phagocytic proteins in order to avoid macrophage clearance.

One well-known interaction that suppresses macrophage responses against cancer cells occurs between CD47 and the immunoreceptor signal regulatory protein-α (SIRPα). CD47 is a membrane protein ubiquitously expressed on all human cells and is the main ligand for SIRPα on macrophages among other cell types. This interaction initiates a cascade of protein phosphorylation, dephosphorylation, and cytoskeleton rearrangement leading to inhibition of phagocytosis. CD47 blockade on human tumor cells with antibodies, such as B6H12 in mouse models and Hu5F9 in the clinic, serves as the predominant therapeutic approach in inhibiting the CD47-SIRPα axis. However, antibodies are very large proteins, often comprising over 1,000 amino acids, that possess additional activities, including possible interactions with Fc receptors on macrophages and with proteins in the complement cascade, which add complexity to safety and efficacy profiles. In addition, anti-CD47 binds CD47 that is expressed on all cells (for example, red blood cells), resulting in the need for very high doses of antibody infusions and resulting in toxic side effects (for example, anemia).

SIRPα is more restricted in expression than CD47, and SIRPα blockade is a potentially safer alternative relative to CD47 blockade. Indeed, data from mice demonstrate that anti-SIRPα blockade with antibodies can be as effective against human tumor growth as CD47 depletion. However, antibodies directed towards SIRPα have general limitations such as those cited above.

Thus, there remains a need in the art for methods and compositions that can efficiently enhance macrophage-mediated phagocytosis of target cells by inhibiting the SIRPα/CD47 interaction in a way that maximizes efficacy and minimizes off-target interactions and side effects including toxicity. The present invention addresses this need.

### BRIEF SUMMARY OF THE DISCLOSURE

The invention of the present disclosure includes methods and compositions that can efficiently enhance macrophage-mediated phagocytosis of target cells by inhibiting the SIRPα/CD47 interaction in a way that minimizes off-target toxicity.

As such, in one aspect, the invention includes a compound of Formula (I), or a salt or solvate thereof:

(BINDERi-LINKERi)iCORE (I)

wherein:
each occurrence of BINDERi is independently a P1 peptide, which comprises the amino acid sequence of SEQ ID NO:1, wherein Xaa5 is Thr or a natural or synthetic amino acid that is not Thr:

Val Thr Glu Leu Xaa5 Arg Glu Gly (SEQ ID NO:1)
each occurrence of LINKERi is independently a linker;
CORE is a moiety independently covalently bound to each (BINDERi-LINKERi) through LINKERi;
‘i’ is an integer equal to or greater than 2.

In certain embodiments, ‘i’ is 2, 3, 4, 5, 6, 7, 8, 9, 10, 11, or 12.

In certain embodiments ‘i’ is 2 and the compound, or a salt or solvate thereof, is:

(BINDER1-LINKER1)-CORE-(LINKER2-BINDER2) (I-1).

In certain embodiments, ‘i’ is 3 and the compound, or a salt or solvate thereof, is:

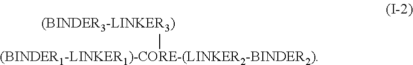

In certain embodiments, ‘i’ is 4 and the compound, or a salt or solvate thereof, is:

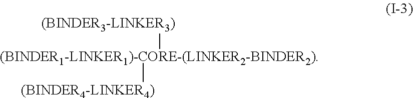

In certain embodiments, Xaa5 is less polar than Thr.

In certain embodiments, Xaa5 is Thr, Ser, Phe, Val, Ala, Leu, Ile, Pro, or Met.

In certain embodiments, Xaa5 is not Thr.

In certain embodiments, in P1 the N-terminus of the amino sequence of SEQ ID NO: 1 is directly coupled with the C-terminus of an amino acid or peptide that is not Gly-Asn-Tyr-Thr-Cys-Glu, Asn-Tyr-Thr-Cys-Glu, Tyr-Thr-Cys-Glu, Thr-Cys-Glu, Cys-Glu, Thr-Glu, or Glu.

In certain embodiments, in P1 the C-terminus of the amino sequence of SEQ ID NO:1 is directly coupled with the N-terminus of an amino acid or peptide that is not Glu-Thr-Ile-Ile-Glu, Glu-Thr-Ile-Ile, Glu-Thr-Ile, Glu-Thr, Glu-Cys, or Glu.

In certain embodiments, at least one BINDERi comprises the amino acid sequence of SEQ ID NOs: 2-4.

In certain embodiments, at least one BINDERi comprises the amino acid sequence of SEQ ID NOs: 6-8.

In certain embodiments, each LINKERi independently comprises a group of Formula (II):

*-(CHR′)m1-X1-(CHR′-CHR′-X2)m2-(CHR′)m3-X3- (II)

wherein:
* indicates the bond between the LINKERi and the BINDERi;
m1, m2, and m3 are independently an integer ranging from 0-100;
each occurrence of X1 and X2 are independently selected from the group consisting of absent (a bond), O, and N(R′);
X3 forms a covalent bond to the CORE and is selected from the group consisting of absent (a bond), C(═O), O, S, and N(R′);
each occurrence of R′is independently selected from the group consisting of hydrogen, optionally substituted C1-C6 alkyl, optionally substituted C3-C8 cycloalkyl, and optionally substituted C3-C8 cycloheteroalkyl.

In certain embodiments, each LINKERi is independently about 5 Å, 6 Å, 7 Å, 8 Å, 9 Å, 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, or 25 Å in length.

In certain embodiments, the CORE is a chemical moiety comprising ‘i’ groups, wherein each group is covalently linked to an individual LINKERi, and wherein each group is independently a carboxylic acid, a primary or secondary amine, a hydroxyl group, a thiol group, or an alkene group.

In certain embodiments, the CORE is an amino acid or a (poly)peptide.

In certain embodiments, the CORE is an amino acid or (poly)peptide comprising at least one of Asp, Glu, Lys, Arg, Ser, Thr, Orn, and Cys.

In certain embodiments, the compound of any of the above aspects or any aspect or embodiment disclosed herein is nS-FF, nS-VV, or nS-F4.

In another aspect, the invention includes a pharmaceutical composition comprising the compound of any of the aspects or embodiments disclosed herein and at least one pharmaceutically acceptable carrier.

In another aspect, the invention includes method of inhibiting biological activity of a signal regulatory protein alpha (SIRPα) , the method comprising contacting the SIRPα with the compound of any of above aspects or any aspect or embodiment disclosed herein.

In certain embodiments, the SIRPα is expressed on the surface of a macrophage.

In certain embodiments, the macrophage is in vivo in a mammal.

In another aspect, the invention includes a method of enhancing phagocytosis by a macrophage, the method comprising contacting the macrophage with the compound of any of the above aspects or any aspect or embodiment disclosed herein.

In certain embodiments, the contacting inhibits at least in part the biological activity of a signal regulatory protein alpha (SIRPα) expressed on the surface of the macrophage.

In certain embodiments, the contacting takes place in the vicinity of a cell to be phagocytized by the macrophage.

In certain embodiments, the cell is at least partially coated by opsonizing antibodies.

In certain embodiments, the cell to be phagocytized is cancerous.

In certain embodiments, the method of any of the above aspects or any aspect or embodiment disclose herein is performed in vivo in a subject suffering from cancer.

In certain embodiments, the compound is administered systemically to the subject.

In certain embodiments, the compound is administered to the vicinity of the cancer and/or intratumorally in the subject.

In certain embodiments, the subject is human.

In another aspect, the invention includes a compound of Formula (I), or a salt or solvate thereof:

(BINDERi-LINKERi)iCORE (I)

wherein:
each occurrence of BINDERi is independently a P1 peptide, wherein P1 comprises Cysª-SEQ ID NO: 1-Cysb, wherein Cysª and Cysb are bridged by a disulfide bond resulting in a cyclic peptide and wherein Xaa5 is Thr or a natural or synthetic amino acid that is not Thr;

Val Thr Glu Leu Xaa5 Arg Glu Gly (SEQ ID NO:1)
each occurrence of LINKERi is independently a linker;
CORE is a moiety independently covalently bound to each (BINDERi-LINKERi) through LINKERi;
‘i’ is an integer equal to or greater than 2.

In certain embodiments, ‘i’ is 2, 3, 4, 5, 6, 7, 8, 9, 10, 11, or 12.

In certain embodiments, ‘i’ is 2 and the compound, or a salt or solvate thereof, is:

(BINDER1-LINKER1)-CORE-(LINKER2-BINDER2) (I-1).

In certain preferred embodiments, ‘i’ is 3 and the compound, or a salt or solvate thereof, is:

(BINDER₃-LINKER₃) (BINDER₁-LINKER))-CORE-(LINKER₂-BINDER₂) (I-2).

In certain preferred embodiments, ‘i’ is 4 and the compound, or a salt or solvate thereof, is:

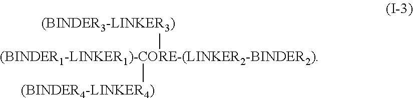

In certain embodiments, Xaa5 is less polar than Thr.

In certain embodiments, Xaa5 is Thr, Ser, Phe, Val, Ala, Leu, Ile, Pro, or Met.

In certain embodiments, Xaa5 is not Thr.

In certain embodiments, in P1 the N-terminus of the amino sequence of SEQ ID NO:1 is directly coupled with the C-terminus of an amino acid or peptide that is not Gly-Asn-Tyr-Thr-Cys-Glu, Asn-Tyr-Thr-Cys-Glu, Tyr-Thr-Cys-Glu, Thr-Cys-Glu, Cys-Glu, Thr-Glu, or Glu.

In certain embodiments, in P1 the C-terminus of the amino sequence of SEQ ID NO: 1 is directly coupled with the N-terminus of an amino acid or peptide that is not Glu-Thr-Ile-Ile-Glu, Glu-Thr-Ile-Ile, Glu-Thr-Ile, Glu-Thr, Glu-Cys, or Glu.

In certain embodiments, at least one BINDERi comprises the amino acid sequence of SEQ ID NOs: 2-4.

In certain embodiments, each LINKERi independently comprises a group of Formula (II):

*-(CHR′)m1-X1-(CHR′-CHR′-X2)m2-(CHR′)m3-X3- (II)

wherein:
* indicates the bond between the LINKERi and the BINDERi;
m1, m2, and m3 are independently an integer ranging from 0-100;
each occurrence of X1 and X2 are independently selected from the group consisting of absent (a bond), O, and N(R′);
X3 forms a covalent bond to the CORE and is selected from the group consisting of absent (a bond), C(═O), O, S, and N(R′);
each occurrence of R′is independently selected from the group consisting of hydrogen, optionally substituted C1-C6 alkyl, optionally substituted C3-C8 cycloalkyl, and optionally substituted C3-C8 cycloheteroalkyl.

In certain embodiments, at least one BINDERi comprises the amino acid sequence of SEQ ID NOs: 6-8.

In certain embodiments, each LINKERi is independently about 5 Å, 6 Å, 7 Å, 8 Å, 9 Å, 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, or 25 Å in length.

In certain embodiments, the CORE is a chemical moiety comprising ‘i’ groups, wherein each group is covalently linked to an individual LINKERi, and wherein each group is independently a carboxylic acid, a primary or secondary amine, a hydroxyl group, a thiol group, or an alkene group.

In certain embodiments, the CORE is an amino acid or a (poly)peptide.

In certain embodiments, the CORE is an amino acid or (poly)peptide comprising at least one of Asp, Glu, Lys, Arg, Ser, Thr, Orn, and Cys.

In certain embodiments, the compound of the above aspects or any aspect or embodiment disclosed herein is nS-FF, nS-VV, or nS-F4.

In another aspect, the invention includes a pharmaceutical composition comprising the compound of any of the above aspects or any aspect or embodiment disclosed herein and at least one pharmaceutically acceptable carrier.

In another aspect, the invention includes a method of inhibiting biological activity of a signal regulatory protein alpha (SIRPα), the method comprising contacting the SIRPα with the compound of any of the above aspects or any aspect or embodiment disclosed herein.

In certain embodiments, the SIRPα is expressed on the surface of a macrophage.

In certain embodiments, the method of claim 52, wherein the macrophage is in vivo in a mammal.

In another aspect, the invention includes a method of enhancing phagocytosis by a macrophage, the method comprising contacting the macrophage with the compound of any of the above aspects or any aspect or embodiment disclosed herein.

In certain embodiments, the contacting inhibits at least in part the biological activity of a signal regulatory protein alpha (SIRPα) expressed on the surface of the macrophage.

In certain embodiments, the contacting takes place in the vicinity of a cell to be phagocytized by the macrophage.

In certain embodiments, the cell is at least partially coated by opsonizing antibodies.

In certain embodiments, the cell to be phagocytized is cancerous.

In certain embodiments, the method of the above aspects or any aspect or embodiment disclosed herein is performed in vivo in a subject suffering from cancer.

In certain embodiments, the compound is administered systemically to the subject.

In certain embodiments, the compound is administered to the vicinity of the cancer and/or intratumorally in the subject.

In certain embodiments, the subject is human.

### BRIEF DESCRIPTION OF THE DRAWINGS

The following detailed description of specific embodiments of the invention will be better understood when read in conjunction with the appended drawings. For the purpose of illustrating the invention, exemplary embodiments are shown in the drawings. It should be understood, however, that the invention is not limited to the precise arrangements and instrumentalities of the embodiments shown in the drawings.

FIG. 1 illustrates nano-Self (nS) peptides inspired by CD47′s site of interaction with SIRPα. The nS-wt peptide consists of 8 amino acid residues, and point mutations at a Thr residue (red in sequence) generate nS peptides as potential antagonists. Bivalent peptides were constructed by linking their monomer counterparts through their respective C-termini. Table: Names and sequences of nS peptides.

FIGS. 2A-2G demonstrate that multivalent nano-Self peptides enhance human macrophage phagocytosis of opsonized targets. FIG. 2A: Schematic of the phagocytosis assay. RBC and K562 erythroleukemia cell opsonization and the treatment of the macrophages with the nS peptides are done separately. Afterwards, opsonized cells are added to the macrophages, and then phagocytosis is measured by counting macrophages with internalized target cells. Addition of nS peptides effectively increases phagocytosis of opsonized cells per fluorescence microscopy images (scale bar: 25 μm). FIG. 2B: Sketch of the tetravalent nS-F4 which consists of a core lysine coupled through both amine functional groups to two lysine residues giving rise to four reactive amine groups. Four simultaneous couplings of the nS-F sequence results with the tetravalent scaffold. FIG. 2C: Incubating various concentrations of the nS peptides with macrophages results in varying levels of macrophages that internalize at least one opsonized RBC. Relative to nSwt, substitutions of the key Thr enhanced phagocytosis as did multivalency. Scrambled nSX or nS-XX peptides do not have effects on phagocytosis. Phagocytosis by mouse macrophages is also affected by nS-wt, albeit not as much as with human macrophages. Baseline phagocytosis of opsonized red blood cells is represented by the red data points. Microscopy fields were selected randomly and at least 200 macrophages were analyzed per condition (n=3±SEM). FIG. 2D: Saturating macrophages with nS-FF, nS-F and nS-VV enhances phagocytosis of opsonized RBCs significantly by an additional ˜10-20% relative to anti-CD47 treatment of opsonized RBCs. nS-wt is least effective but gives the same result as anti-CD47 and is greater than just opsonized RBCs. Microscopy fields were selected randomly and at least 200 macrophages were analyzed per condition (n=3±SEM; * denotes p<0.05 relative to CD47-blocked and opsonized RBCs). FIG. 2E: Effects on phagocytosis of K562 erythroleukemia cells are enhanced by nS-FF and nSF4. The increase in valency appears to have a slight effect at high concentration of peptide but phagocytosis levels were largely similar between bivalent and tetravalent peptides. FIG. 2F: Saturating macrophages with nS-FF and nS-F4 significantly increases phagocytosis of opsonized K562 cancer cells by about two-fold relative to opsonized and CD47 blocked cells with only opsonization effect observed with nS-XX addition. FIG. 2G: Schematic of potential mechanism by which the nS peptides engage and enhance phagocytosis. Left panel: CD47-expressing cells signal ‘self’ to macrophage through engagement of CD47 on the cell surface with SIRPα on the macrophage, increasing pTyr signals, and overriding pro-phagocytic signaling from the opsonizing anti-RBC IgG antibody ultimately inhibiting phagocytosis. Right panel: nS peptides engage with SIRPα, inhibiting trans binding of CD47 on opsonized cells and inhibiting cis binding on the macrophages, leading to increased phagocytosis.

FIGS. 3A-3B demonstrate that the binding and signaling of nano-Self peptides is consistent with SIRPα inhibition. FIG. 3A: Representative fluorescent images of nS-F-FAM fluorescence inhibition after addition of multivalent nS peptide inhibitors. FIG. 3B: Bivalent peptides show higher affinity towards macrophages by outcompeting the binding of monovalent nS-F-FAM (n=2±SEM; * denotes p<0.05 relative to control).

FIGS. 4A-4B are a series of charts illustrating that the disordered structure with some hairpin folding suggests induced fit mechanism into SIRPα binding pocket. FIG. 4A: CD spectra of nS-FF, nS-VV, nS-XX at 5 and 90 ° C. Arrows indicate ellipticity points at 215-220 nm and 195 nm, respectively, suggestive of some β-hairpin fold which is lost in nS-XX. FIG. 4B: Difference plots show nS-FF and nS-VV are in agreement in terms of structure consistent with phagocytosis results.

FIGS. 5A-5H are a series of graphs demonstrating the synthesis and purity of nano-Self peptides are characterized and confirmed by analytical HPLC and MALDI-TOF mass spectrometry. All peptides used in this assay were run on an analytical HPLC to determine purity the nS peptides. All analytical traces show one major peak for each peptide. The peptides were all characterized by MALDI-TOF mass spectrometry. Single main peaks appear for the correct mass for all peptides.

FIGS. 6A-6B are a series of graphs illustrating that opsonization titer determines optimal amount of opsonin needed for assays. FIG. 6A: Varying concentrations of opsonin were incubated with CD47-blocked RBCs to determine the optimal amount of opsonization necessary for optimal phagocytosis. J774A.1 and THP-1 macrophages responded differently to RBC opsonization which may be a result of the polyclonal antibody and the way it engages with FcRs on the respective macrophages. The red point indicates the concentration of opsonin used in the phagocytosis assays which is 133 nM for both. RBCs are engulfed in an opsonin concentration-dependent manner even in the absence CD47 blockade (shown in green) suggesting some nonspecific FcR activation by the anti-CD47 antibody. FIG. 6B: Similar to trends observed with THP-1 phagocytosis, treating mouse J774A.1 macrophages with nS peptides results in enhanced engulfment of opsonized RBCs.

FIG. 7 is a series of micrographs illustrating that nano-Self peptides bind to soluble SIRPα. Polystyrene beads coated with streptavidin incubated with or without biotinylated nS-wt or nS-V. SIRPα was stained with FITC-labeled primary antibody and fluorescence is only observed in the presence of biotinylated peptide.

FIG. 8 is a series of micrographs illustrating that nano-Self peptides bind to membrane bound SIRPα and are also internalized by human and mouse macrophages. Fluorescence images of peptides bound to J774A. 1 and THP-1 macrophages. Fluorescence is observed on the edges of the macrophages but not on the entire membrane. Low signal of nS-X-FAM is indicative of internalization. High signal resulting from the remaining peptides is cumulative signal from binding and internalization.

FIG. 9 is a graph demonstrating the binding of nano-Self peptides to SIRPα is considerably weak. Peptide binding affinities to membrane expressed SIRPα on fixed THP-1 macrophages. Fluorescence of FAM-labelled peptides was measured using fluorescence microscopy. Binding affinities (kd) were obtained by fitting one site—specific binding saturation plots.

FIGS. 10A-10C are a series of graphs showing the apparent affinities of nano-Self peptides decrease on live cells due to macrophage internalization. Peptide binding affinities to membrane expressed SIRPα on live (FIG. 10A) J774A.1 and (FIG. 10B) THP-1 macrophages. (FIG. 10C) The fluorescence resulting from internalized peptides was accounted for by subtracting the fluorescence of the scrambled nS-X-FAM peptide, resulting with corrected binding affinities. Fluorescence of FAM-labelled peptides was measured using fluorescence microscopy. Binding affinities (kd) were obtained by fitting one site - specific binding saturation plots.

FIG. 11 is a series of micrographs that illustrates the observation that nano-Self peptides are internalized by human and mouse macrophages at high concentrations. Confocal z-stack images of J774A.1 and THP-1 macrophages incubated with FAM-labelled peptides. In addition to membrane binding, internalization of nS-wt occurs more readily at higher concentrations leading to higher fluorescence intensity. At low (1 μM) concentrations, the peptide is mainly internalized with minimal membrane staining hence the decrease in fluorescence.

FIGS. 12A-12B are a series of graphs illustrating that nano-Self peptides conform to some β-hairpin structure but mainly random coil. FIG. 12A: CD spectra of monovalent nS peptides. FIG. 12B: FT-IR spectra of bivalent nS peptides. Main peak appears near 1650 cm-1 which is a characteristic peak shared by both random coil and B-turn peptides.

FIG. 13 is a non-limiting illustration of a tetravalent compound of the invention . Bivalent peptides were linked by their respective C-terminal Lys residues to an anchored Lys residue off-resin.

FIG. 14 is a series of graphs demonstrating the synthesis and purity of the tetravalent nano-Self peptide The analytical trace and MALDI-TOF spectrum each show one major peak for nS-F4 indicating successful and pure synthesis.

FIG. 15 illustrates that PMA differentiated THP-1 cells share identical gene profiles as primary macrophages for key, pathway-relevant factors (Top) Microarray gene expression analysis verification that PMA differentiated THP-1 macrophages express several key macrophage factors at similar levels as primary mouse macrophages (i.e. Sirpa, the Integrin (Itg) genes, Fcgr genes, and SHP1 gene Ptpn6), while differing from other hematopoietic and non-hematpoietic cells (K562 and HEK, respectively). Positive control genes are the ubiquitous Cd47 and the widely expressed nonmuscle myosin-II gene, Myh9. Negative control genes are skeletal muscle myosin, Myh1, and melanin synthesis gene, Tyrp1. (Bottom) Dendrogram from hierarchical clustering analysis validating similar profiles between marrow-derived mouse macrophages and PMA-differentiated THP-1 macrophages while clearly distinct from other cell types.

FIG. 16 is a table illustrating that the Treatment of human macrophages with multivalent nano-Self peptides enhances phagocytosis levels significantly relative to peptide and antibody controls All conditions were compared with each other to determine which conditions were significantly different (* denotes p<0.05 for statistical significance between conditions). Both multivalent peptides enhanced macrophage phagocytosis significantly compared to all control conditions.

FIGS. 17A-17B illustrates that using optimal concentration of opsonin allows for comparison of macrophage phagocytosis. Varying concentrations of anti-RBC opsonin incubated with K562 cells (FIG. 17A) to determine the optimal amount of opsonization necessary for optimal phagocytosis. Human and mouse macrophages responded differently to RBC opsonization, which may be a result of the polyclonal antibody and how it engages with FcRs on the respective macrophages. The optimal concentration for phagocytosis assays was selected to be 133 nM (red point) because it gave a reasonable phagocytosis response far from baseline and saturation. FIG. 17B. Treating mouse macrophages with nS peptides results in enhanced engulfment of opsonized RBCs.

FIG. 18 shows CD47 expression on RBCs and K562 cancer cells Flow cytometry quantitation of primary fluorescent anti-CD47 antibody. Samples were analyzed the same day using the same voltage settings on the flow cytometer. Higher numbers of CD47 molecules are present on the surface of K562 cells when compared to RBCs.

FIG. 19 shows a CD47-Fc binding curve. Varying concentrations of CD47-Fc incubated with human THP-1 macrophages and measured by anti-Fc fluorescence. Apparent Kd was determined to be 0.46 μg/mL.

FIG. 20 illustrates that binding of nano-Self peptides is consistent with SIRPα inhibition Representative fluorescence microscopy images of CD47-Fc inhibition by multivalent nSFF and nS-F4 peptides. Anti-CD47 and CD47-Fc were incubated together prior to their addition to macrophages. All conditions were compared to saturating concentration of CD47-Fc.

Quantitation was done by measuring anti-Fc fluorescence (n=2±SEM; * denotes p<0.05; scale bar: 50 μm).

FIGS. 21A-21B illustrate that bivalent nano-Self peptides suppress macrophage phosphotyrosine levels consistent with disruption of ‘Self’ signaling in cis. FIG. 21A. Basal levels of pTyr signal are observed in isolated macrophages. pTyr signal is suppressed upon the addition of nS-FF. pTyr levels decrease in isolated macrophages when either CD47 is blocked or nS peptides are added. This inhibition of phosphorylation is not observed when macrophages are treated with nS-X, supporting that the loss of phosphorylation signal is due to inhibition of CD47-SIRPα binding. Multivalent nS-FF suppressed pTyr at nanomolar concentrations, whereas no effect was observed with monovalent nS-F, consistent with higher affinity of multivalent nS peptides (n=3±SEM; * denotes p <0.05 relative to control; scale bar: 25 μm). FIG. 21B. Schematic representing potential mechanism of nS peptides antagonizing the macrophage checkpoint. Anti-CD47 binds CD47 on the surface of macrophages, inhibiting its binding to SIRPα binding thus suppressing pTyr. Addition of nS peptides replicates the same effect of suppressing pTyr to similar levels as anti-CD47 blockade, consistent with -and providing an explanation for -the phagocytosis results. This suggests disruption of the CD47-SIRPα axis.

FIG. 22 illustrates that avidity of SIRPα appears to scale with the increase of nano-Self Multivalency Tetravalent nS-F4 was more potent in inhibiting monovalent nS-F-fluor association with macrophages than bivalent nS-FF suggesting multivalency increases avidity of the nS peptides to SIRPα.

FIGS. 23A-23D illustrate that pre-clinical assessments indicate nS-FF is safe in vivo Phase 1 pre-clinical trial: Intravenous injections of nS-FF were done for four consecutive days followed by blood withdrawal 24 hours after last injection. Blood parameters show that nS-FF at 1 mg/kg is safe, with no anemia or weight loss.

FIGS. 24A-24B demonstrate that the sequence of the cyclic nano-Self peptide is not found in nature suggesting low immunogenicity FIG. 24A: The chemical structure of nS-Cyc consists of the 8-amino acid sequence of nS-wt bridged through a disulfide bond at the termini. Synthesis of the construct was verified by MALDI-TOF mass spectrometry indicating the oxidation of the cysteine thiols forming a disulfide bond. Inset table: The sequences of the linear and cyclic nS-peptides are shown. Although the linear peptides are found primarily in bacteria, the nS-peptides are not likely to elicit immune responses. Immunogenicity scores were generated using the IEDB Analysis Resource. FIG. 24B: Sequence search analysis reveals that there are no proteins expressed by any organism that contain the exact sequence of the cyclic nS peptides. The closest sequence to nS-Cyc is found in a toxic plant; however, the aligned protein seems unrelated to the toxicity of the plant. The residues that do not match the nS-Cyc sequences the subject protein sequence are highlighted in red.

FIGS. 25-25C illustrate that RBC phagocytosis by human and mouse macrophages increases with the addition of nS-Cyc A. FIG. 25A cartoon representation of the phagocytosis assay performed. Briefly, adherent human or mouse macrophages are incubated with either linear or cyclic nS-peptides and then fed opsonized human RBCs. Internalization of RBCs is analyzed by fluorescence microscopy. FIG. 25B: Phagocytosis levels in human (i) and mouse (ii) macrophages are enhanced with the addition of linear and cyclic nS-peptides. nS-Cyc appears to have a greater effect in perturbing the CD47-SIRPα interaction compared to linear nS-wt in human macrophages, but is not as potent as nS-F. This suggests that cyclization increases potency, but the sequence is also an important component of activity. Values are all normalized relative to anti-CD47 blockade and anti-RBC opsonization (+,+). FIG. 25C: Addition of low concentration of nS-Cyc results with macrophages internalizing at least one opsonized RBC, with levels of phagocytic macrophages slightly greater than linear nSwt. Although efficacy of nS-Cyc is less in mouse macrophages than in human, it is still roughly 20-fold more active than the linear counterpart.

FIG. 26 illustrates a non-exclusive example of an nS-F-Cyc peptide in addition to a bivalent variant.

FIGS. 27A-27B illustrate that successful transduction of CD47-GFP variants can be used for binding assays. FIG. 27A: Viral transduction and expression of wild type CD47-GFP and various mutants on HEK-293 cells was confirmed with fluorescence microscopy. The T102S mutation is based on the single point mutation found in the mouse CD47 β-hairpin that interacts with SIRPα. T102F and T102V are based the nS-peptides that showed the most potent activity in prior phagocytosis and inhibition studies. FIG. 297: Binding of SIRPα-Fc fusion protein to the mutated CD47 variants will be evaluated as well as efficacy of the nS-Cyc peptides to inhibit SIRPα binding to CD47.

FIGS. 28A-28B illustrate that nano-Self peptides which are designed based on the human CD47-SIRPα interaction with cyclic variants not found in nature. FIG. 28A. Derived from CD47, the 8-amino acid nano-Self peptide region in CD47 (blue) interacts with 3 (red) out of the 4 loops in SIRPα (grey) that make up the binding pocket which amounts to roughly 40% of all contact residues between the two proteins. PDB: 2JJS. FIG. 28B. The critical polar Thr residue in nS-wt (blue) is inserted into a hydrophobic core in the SIRPα binding pocket (red). Removing the polar Thr to a hydrophobic Phe, which can also engage in pi-stacking interactions with Phe⁷⁴ away from the SIRPα binding pocket, may potentially enhance binding efficiency of the peptide.

FIGS. 29A-29D illustrate that phagocytosis of antibody-opsonized mouse and human target cells is enhanced in the presence of linear and cyclic nano-Self peptides. FIG. 29A. SIRPα knockout (KO) or parental (WT) mouse melanoma B16 cells are anti-TYRPI opsonized and phagocytosed by mouse bone marrow derived macrophages in the presence of the nano-Self peptides (top). Internalization of B16 cells (green) is distinguished from adherent cells by the physical rounding up the cells inside the macrophages since B16 cells can attach and spread on plastic (bottom) (scale bar=25 μm). FIG. 29B. Phagocytosis of both WT and SIRPα KO B16 cells is enhanced significantly by the linear and cyclic nS-F peptides. nS-wt had similar effects as anti-mCD47 with no effect of nS-X on phagocytosis. Cis interactions between CD47 and SIRPα on B16s effects phagocytosis levels with less phagocytic macrophages against SIRPα KO B16s due to more accessible CD47 molecules on their membranes. Hyper-phagocytosis is also observed when BMDMs are treated with potent nS-F and cyclic variants. At least 200 macrophages were analyzed per condition. (* denotes p<0.05 relative to anti-mCD47 and anti-TYRP1 treated B16s). FIG. 29C. The in-vitro phagocytosis of red blood cells is achieved by disrupting the CD47-SIRPα axis with nano-Self peptides and supplying a strong phagocytic stimulus via macrophage FcR-binding to IgG antibodies (top). Phagocytosed (red) and uninternalized red blood cells (green) are visualized using fluorescence microscopy as seen in the representative overlays (bottom) (scale bar=25 μm). FIG. 29D. Phagocytosis levels are enhanced when macrophages are incubated with saturating concentrations of nano-Self peptides as does CD47-blockade of red blood cells. Mutating the critical Thr residue to Phe in the linear (nS-F) and cyclic (nS-F-Cyc) variants increases macrophage phagocytosis by roughly 25% relative to nS-wt, while no effect is observed in the presence of scrambled nS-X. Hyper-phagocytosis is observed for F-variants due to simultaneous cis and trans inhibition of the CD47-SIRPα axis. At least 200 macrophages were analyzed per condition. (* denotes p<0.05 relative to anti-CD47 and anti-RBC treated RBCs).

FIGS. 30A-30B illustrate that nano-Self peptides minimize target cell adhesion to macrophage leading to increased phagocytosis. FIG. 30A. Opsonized RBCs adhere to the surface of macrophages through IgG-FcR mediated binding in addition to possible CD47-SIRPαbinding that silences uptake of RBCs. FcR-mediated activation leads to macrophage uptake in the presence of the nS peptides that also effectively block CD47-SIRPα binding. The % Adhered RBCs is measured from the total RBCs imaged, which are either adherent to a macrophage or else internalized. FIG. 30B. Opsonized B16 cells adhere to macrophages through FcR-binding as well as CD47-SIRPα binding but also compete for the plastic surface. Once attached and spread on plastic, the macrophage cannot strip the cancer cell off. B16 cells that interact with macrophages while in suspension are engulfed when nano-Self peptides are added. Insert: a BMDM (red fluorescence, black outline) contacting an adhered, opsonized B16 cell (green fluorescence, white outline). Non-linear regression fits (see text):
RBC: yₘₐₓ=30; m=4.95; A^{m}=7.46'10¹³; B^{m}=1.37×10¹¹; p=0.03
B16: yₘₐₓ=27.8; m=4.94; A^{m}=9.26×10¹²; B^{m}=4.69×10¹⁰; P_{WT}=0.04; p_{KO}=0.02

FIGS. 31A-31C illustrate that engineered nano-Self peptides are potent at nanomolar concentrations for human macrophage phagocytosis but much weaker with primary mouse macrophages. FIG. 31A. Saturating human macrophages with the various nano-Self peptides results with maximum macrophage internalization of opsonized RBCs. At nanomolar concentrations, the nano-Self peptide variants are effective at promoting near-maximum macrophage phagocytosis with significantly lower efficacy for nS-wt. FIG. 31B. Estimates for effective concentrations of the nS peptides function in mouse BMDMs for phagocytosing B16 cells. FIG. 31C. SIRPα sequence analysis between human and various mouse species reveals significant changes in the binding loop sequences (underlined residues). All highlighted residues are directly involved in binding the nS-wt region of CD47. Green highlighted residues are conserved relative to human SIRPα variant 2. Red highlighted residues are non-conserved mutations disrupting H-binding.

FIG. 32 illustrates that the design of cyclic nano-Self accounts for the presence of negatively charged residues in the wildtype sequence.

FIG. 33 illustrates that the design of cyclic nano-Self accounts for the presence of negatively charged residues in the wildtype sequence.

FIG. 34 illustrates analytical confirmation of purity and characterization of the nano-Self peptides.

FIG. 35 illustrates B16 SIRPα knockout validation.

FIG. 36 illustrates that adhesion of opsonized RBCs on macrophages surfaces inversely correlates with nS peptide concentration.

FIG. 37 illustrates that low activity of bivalent nS-FF in primary mouse macrophages suggests structure and sequence are key for potent nS peptide binding.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, selected materials and methods are described herein. In describing and claiming the present invention, the following terminology will be used.

It is also to be understood that the terminology used herein is for the purpose of describing particular embodiments only, and is not intended to be limiting. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated, synthesized or can be derived from a biological sample. Such a biological sample can include, but is not limited to a tissue sample, a tumor sample, a cell or a biological fluid.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

As used herein, the terms “effective amount” and “pharmaceutically effective amount” refer to a nontoxic but sufficient amount of an agent or drug to provide the desired biological result. That result can be reduction and/or alleviation of the signs, symptoms, or causes of a disease or disorder, imaging or monitoring of an in vitro or in vivo system (including a living organism), or any other desired alteration of a biological system. An appropriate effective amount in any individual case may be determined by one of ordinary skill in the art using routine experimentation.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

The term “immune response” as used herein is defined as a cellular response to an antigen that occurs when lymphocytes identify antigenic molecules as foreign and induce the formation of antibodies and/or activate lymphocytes to remove the antigen.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

An “individual”, “patient” or “subject”, as that term is used herein, includes a member of any animal species including, but are not limited to, birds, humans and other primates, and other mammals including commercially relevant mammals such as cattle, pigs, horses, sheep, cats, and dogs. Preferably, the subject is a human.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Isolated” means altered or removed from the natural state through the actions of a human being. For example, a nucleic acid or a protein naturally present in a living animal is not “isolated,” but the same nucleic acid or protein partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human. “Parenteral” administration of an immunogenic composition includes, e.g., subcutaneous (s.c.), intravenous (i.v.), intramuscular (i.m.), or intrasternal injection, or infusion techniques. As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that can be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof. Pharmaceutically unacceptable salts may nonetheless possess properties such as high crystallinity, which have utility in the practice of the present invention, such as for example utility in process of synthesis, purification or formulation of compounds useful within the methods of the invention. Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric, galacturonic acid, glycerophosphonic acids and saccharin (e.g., saccharinate, saccharate). Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, the terms “protein”, “peptide” and “polypeptide” are used interchangeably, and refer to a compound comprised of amino acid residues covalently linked by peptide bonds. The term “peptide bond” means a covalent amide linkage formed by loss of a molecule of water between the carboxyl group of one amino acid and the amino group of a second amino acid. A protein or peptide must contain at least two amino acids, and no limitation is placed on the maximum number of amino acids that may comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Proteins” include, for example, biologically active fragments, substantially homologous proteins, oligopeptides, homodimers, heterodimers, variants of proteins, modified proteins, derivatives, analogs, and fusion proteins, among others. The proteins include natural proteins, recombinant proteins, synthetic proteins, or a combination thereof. A protein may be a receptor or a non-receptor.

As used herein, the term “salt” embraces addition salts of free acids or free bases that are compounds useful within the invention. Suitable acid addition salts may be prepared from an inorganic acid or from an organic acid. Examples of inorganic acids include hydrochloric, hydrobromic, hydriodic, nitric, carbonic, sulfuric, phosphoric acids, perchloric and tetrafluoroboronic acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable base addition salts of compounds useful within the invention include, for example, metallic salts including alkali metal, alkaline earth metal and transition metal salts such as, for example, lithium, calcium, magnesium, potassium, sodium and zinc salts. Acceptable base addition salts also include organic salts made from basic amines such as, for example, N,N′-dibenzylethylenediamine, chloroprocaine, choline, diethanolamine, ethylenediamine, meglumine (N-methyl-glucamine) and procaine. All of these salts may be prepared by conventional means from the corresponding free base compound by reacting, for example, the appropriate acid or base with the corresponding free base.

As used herein, the term “SIRP-a” or “SIRP-α” or “SIRP-alpha” refers to signal regulatory protein-α (also known as signal regulatory protein-a).

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “substantially the same” amino acid sequence is defined as a sequence with at least 70%, preferably at least about 80%, more preferably at least about 85%, more preferably at least about 90%, even more preferably at least about 95%, and most preferably at least 99% homology with another amino acid sequence, as determined by the FASTA search method in accordance with Pearson & Lipman, 1988, Proc. Natl. Inst. Acad. Sci. USA 85:2444-48.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

To “treat” a disease as the term is used herein, means to reduce the frequency or severity of at least one sign or symptom of a disease or disorder experienced by a subject.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl. A non-limiting example is (C₁-C₆)alkyl, particularly ethyl, methyl, isopropyl, isobutyl, n-pentyl, n-hexyl and cyclopropylmethyl.

As used herein, the term “substituted alkyl” means alkyl as defined above, substituted by one, two or three substituents selected from the group consisting of halogen, —OH, alkoxy, —NH₂, —N(CH₃)2, —C(═O)OH, trifluoromethyl, —C═N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(—O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. A non-limiting example is (C₁-C₃) alkoxy, particularly ethoxy and methoxy. As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine. As used herein, the term “cycloalkyl” refers to a mono cyclic or polycyclic non-aromatic radical, wherein each of the atoms forming the ring (i.e. skeletal atoms) is a carbon atom. In certain embodiments, the cycloalkyl group is saturated or partially unsaturated. In other embodiments, the cycloalkyl group is fused with an aromatic ring. Cycloalkyl groups include groups having from 3 to 10 ring atoms. Illustrative examples of cycloalkyl groups include, but are not limited to, the following moieties:

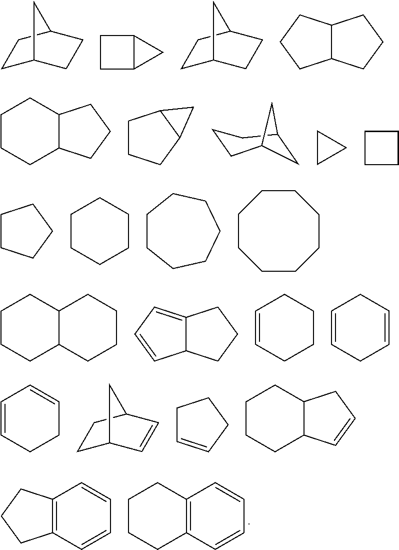

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution can be at any chemically accessible position. In certain embodiments, the substituents vary in number between one and four. In other embodiments, the substituents vary in number between one and three. In yet other embodiments, the substituents vary in number between one and two.

As used herein, the term “optionally substituted” means that the referenced group can be substituted or unsubstituted. In certain embodiments, the referenced group is optionally substituted with zero substituents, i.e., the referenced group is unsubstituted. In other embodiments, the referenced group is optionally substituted with one or more additional group(s) individually and independently selected from groups described herein.

In certain embodiments, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)zalkyl, —C(—O)NH[substituted or unsubstituted alkyl], —C(—O)NH[substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, -N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In other embodiments, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, -OCH₃, -OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, and —C(═O)OH. In yet one embodiment, the substituents are independently selected from the group consisting of C₁₋₆ alkyl, —OH, C₁₋₆ alkoxy, halo, amino, acetamido, oxo and nitro. In yet other embodiments, the substituents are independently selected from the group consisting of C₁₋₆ alkyl, C₁₋₆ alkoxy, halo, acetamido, and nitro. As used herein, where a substituent is an alkyl or alkoxy group, the carbon chain can be branched, straight or cyclic.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

There is renewed interested in developing peptide-based therapies and treatments for disease. Peptides are biocompatible and are less likely to have toxic side effects. There are many peptide agents used daily by patients of various diseases as well as hundreds of other peptides currently being investigated in the clinic. Moreover, peptides are much more soluble at higher molar concentrations than therapeutic antibodies and can be synthesized at high yields and low cost. Antibodies are large macromolecules (>150 kDa) where their production is cumbersome and most of the antibody mass is not needed. Typical costs of lab grade antibodies, such as anti-CD47, anti—SIRPα, and anti-PD1 from leading suppliers are in the range of $100/mg. Clinical grade antibodies such as anti-PD1 currently cost more than $100 k/patient/year.

The present disclosure describes the facile synthesis and application of short peptides that target SIRPα on macrophages and enhance phagocytosis of opsonized red blood cells (opRBCs). These peptides can be further engineered to generate bivalent adjuvants (approximately $2/mg) that dually bind and inhibit SIRPα ultimately increasing potency with effects observed at 20 nM concentrations. This novel proof-of-concept application of CD47-based peptides demonstrates a method to engineer macrophages to target cancer. In this study, these nano—Self (nS) peptides are demonstrated to interact with mouse-derived J774A.1 macrophages, which is a main animal for preclinical tests of anti-cancer efficacy and safety studies. From sequence analysis of SIRPα of various species, critical contact residues with CD47 are conserved among some species relative to human SIRPα. Based on this, the present bivalent nS peptides are expected to bind pig, monkey, and dog macrophages, which are interesting and important species for evaluation of safety and anti-cancer efficacy.

The CD47—SIRPα co-crystal (PDB: 2JJS) shows at least three sites of interaction. As shown herein, 8-amino acid nano—Self (nS) peptides were inspired by this complex interaction and synthesized in multivalent forms to try to exert an effect on phagocytosis. The present disclosure shows that bivalent nS peptides at nanomolar concentrations enhance phagocytic levels beyond target blockade with anti—CD47.

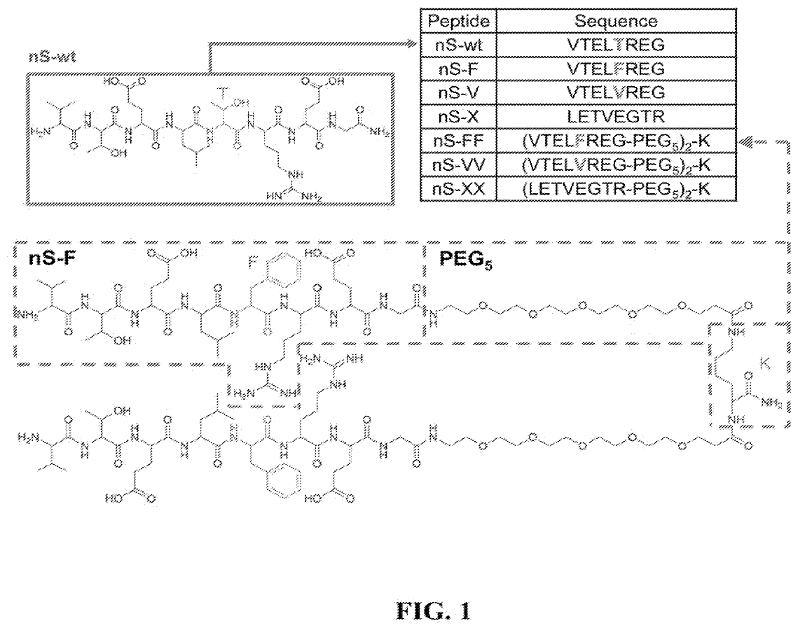

As described herein, seven soluble peptides were synthesized (FIG. 1 and Table 1). From the SIRPα-CD47 crystal structure, T¹⁰² in CD47 points towards a hydrophobic site in SIRPα. The polar T residue in nS-wt was therefore mutated to hydrophobic F or V. A scrambled control (nS-X) was synthesized for comparisons. To test the effects of multivalency, bivalent and tetravalent peptides were synthesized. In certain embodiments, a multivalent peptide has the ability to potentially bind two SIRPα proteins and be a more potent inhibitor. PEG₅ linkers were inserted at the C-termini of both nS-V and nS-F monomers and anchored with a C-terminal Lys residue to generate C-terminally linked nS-V and nS-F peptides. The same was done with nS-X to be used as a control.

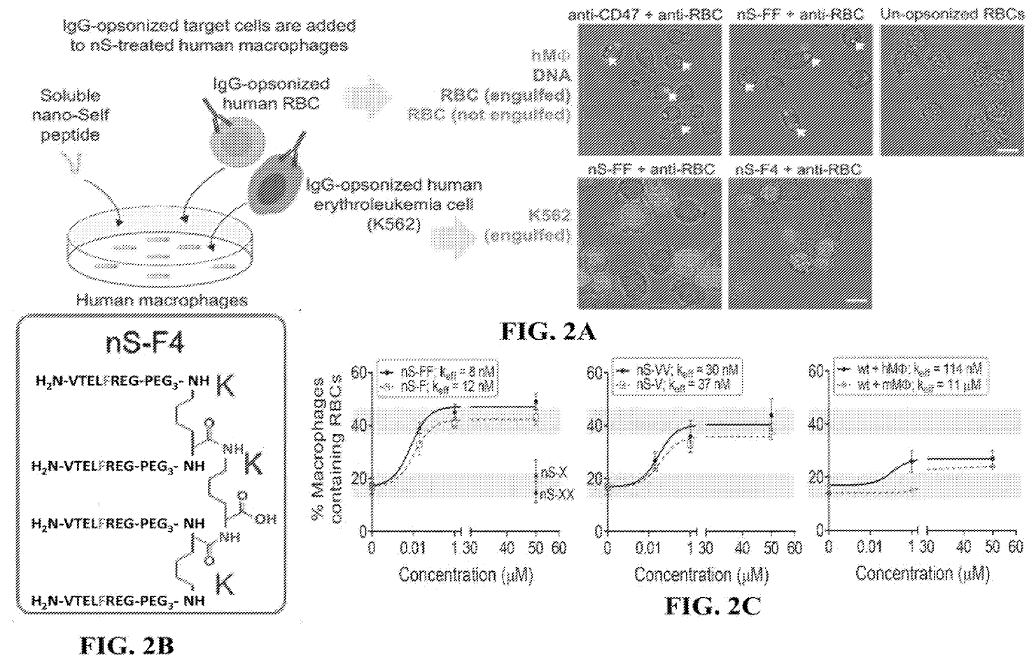

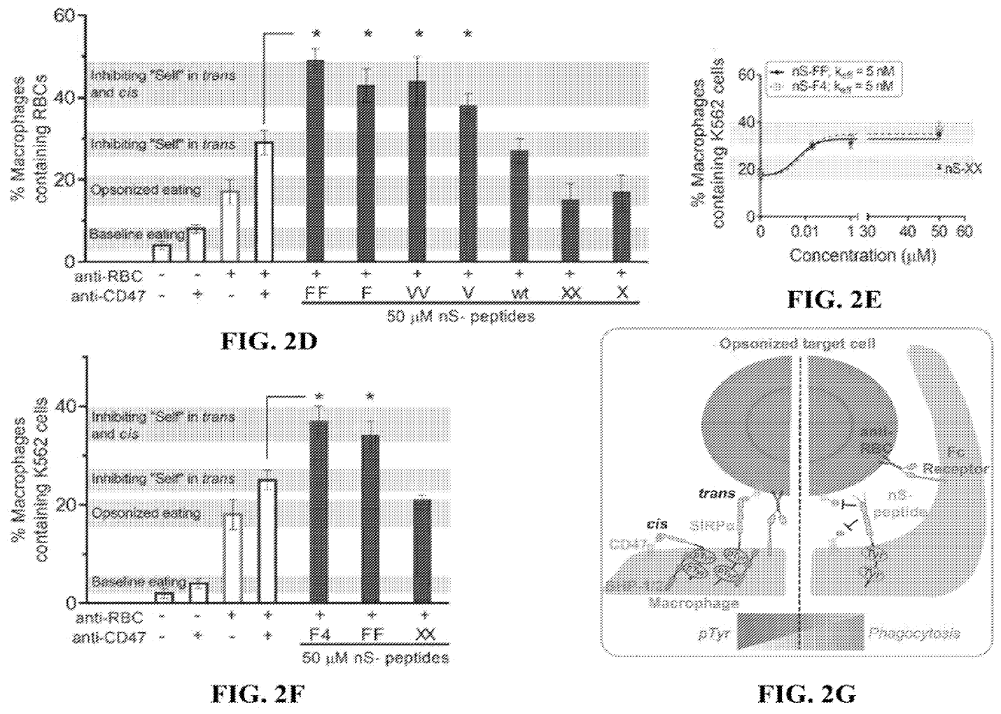

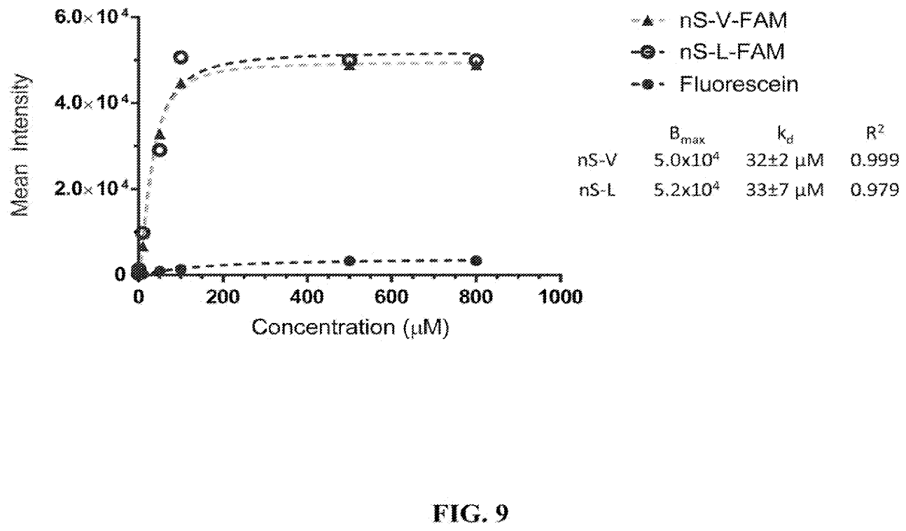

Although the peptides are very short (8 amino acids) compared to the complexity of the multi-site interaction in the CD47—SIRPα co-crystal (PDB: 2JJS), they were effective in enhancing phagocytic levels of both THP-1 and J774A. 1 macrophages (FIGS. 2A-2C and 9A-9B). This result indicates the binding of the peptides to SIRPα on macrophages. These nS peptides bind with enough affinity to the surface of macrophages to prevent the binding of anti-phagocytic CD47 on RBCs to SIRPα on the macrophage leading to RBC engulfment.

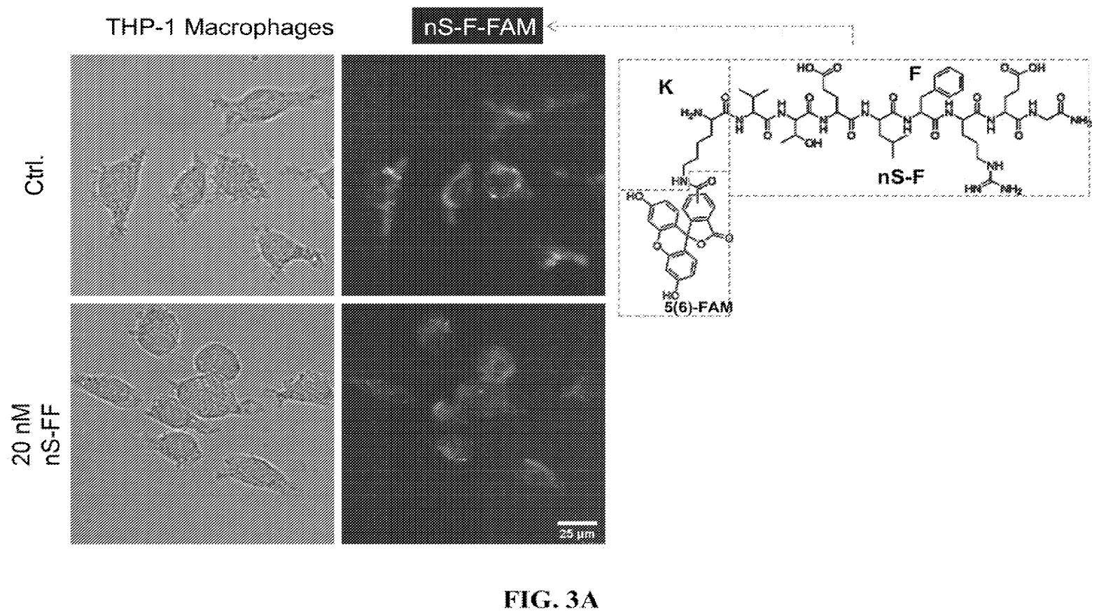

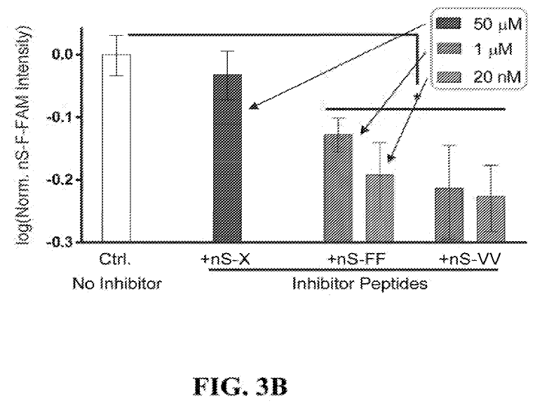

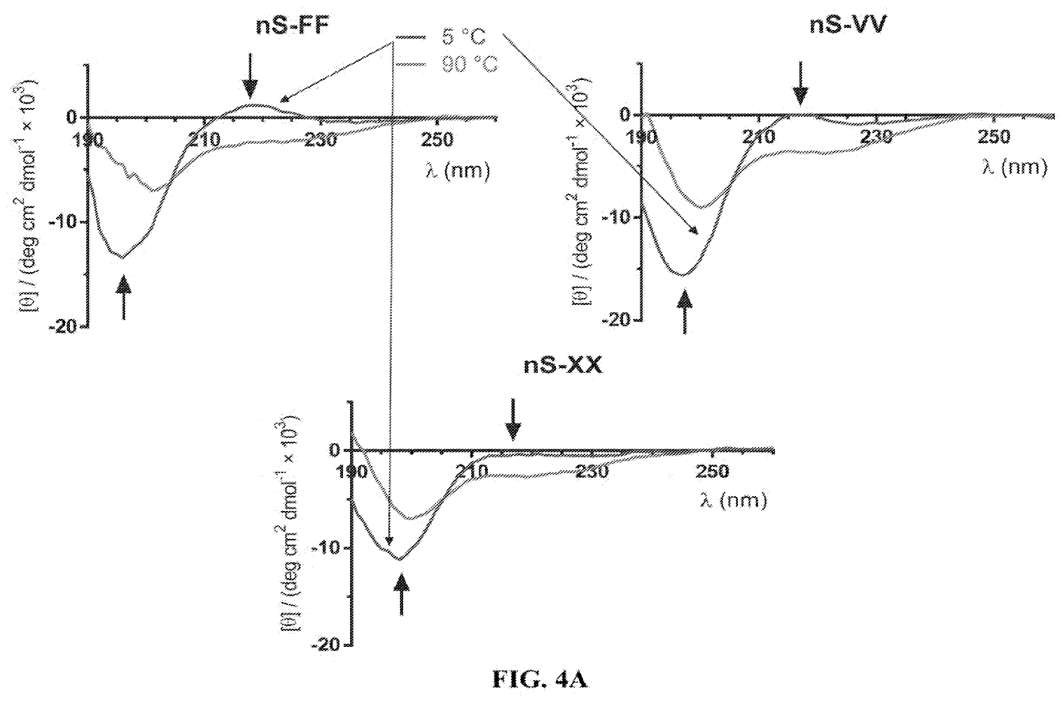

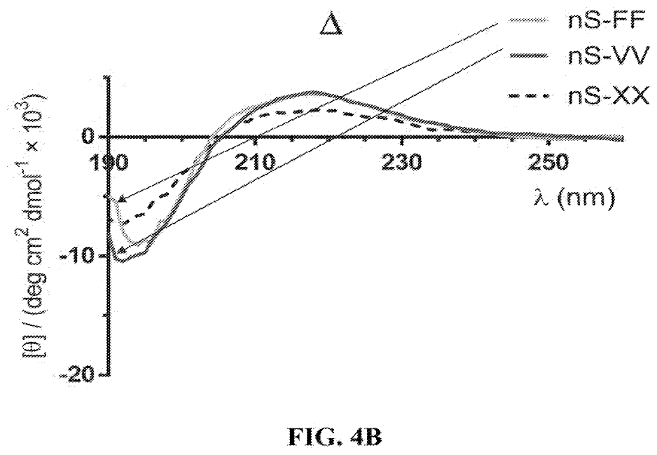

The engulfment of opRBCs by macrophages inhibited with the present peptides was compared to CD47 inhibited opRBCs with B6H12 (anti—CD47). Data obtained by treating the macrophages with the bivalent peptides suggest a more potent and efficacious effect on phagocytosis. These peptides based on CD analysis have some characteristics of β-hairpin structure in solution (FIGS. 4A-4B), which in non-limiting aspects may have contributed to the increase in phagocytic levels in THP-1 macrophages. nS-FF and nS-VV were successful in activating THP-1 to engulf opRBCs at pharmacological relevant concentrations (20 nM). Without wishing to be limited by any theory, this was not observed with nS-F and nS-V where 20 nM treatment did not significantly change the amount of macrophage eating relative to anti-CD47 treatment of opRBCs (FIG. 2B). Without wishing to be limited by any theory, the increased levels of eating may be due to cumulative effects of cis and trans inhibition making the macrophages effectively more hungry for opRBCs. Indeed nS-FF, and not nS-F, suppresses phosphorylation in THP-1 macrophages at 20 nM, indicating SIRPα antagonization and blocking cis binding with CD47 (FIGS. 3C-3D).

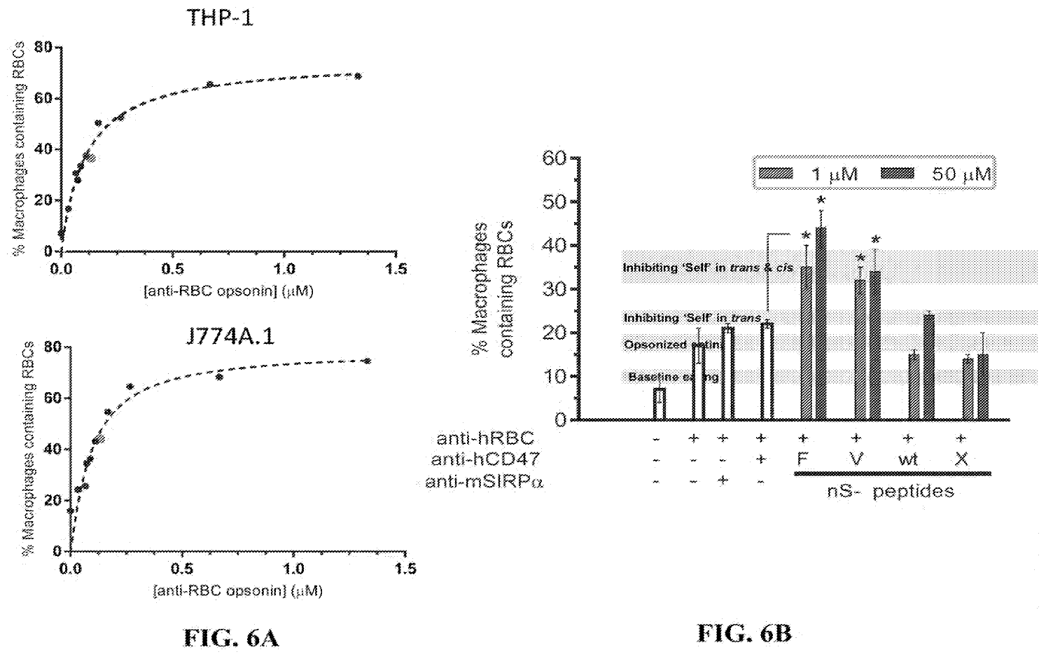

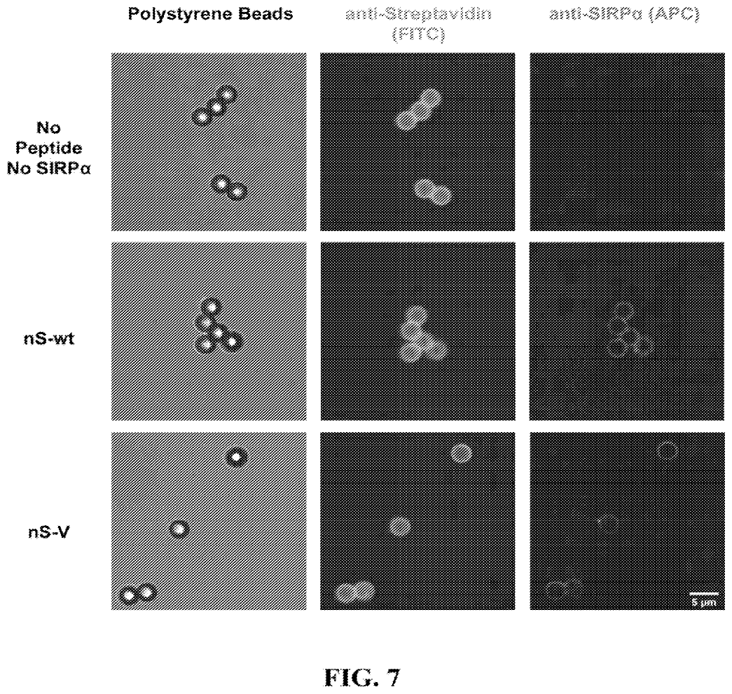

Phagocytosis is enhanced with the mutation of T in nS-wt to F and V, respectively. The bulky and hydrophobic F mutation seemingly has an even higher effect when compared to nS-V. Nevertheless, the mutation of the polar T to hydrophobic V had a positive effect on phagocytosis. nS-wt is the same sequence from the wild type CD47 FG-loop which may not efficiently inhibit cis interactions on the surface of THP-1 macrophages giving similar results as trans inhibition by anti—CD47 blockade of opRBCs. The point mutations seemingly increased binding affinities of the peptides to SIRPα inhibiting both cis and trans interactions between CD47 and SIRPα (FIG. 2B). These observations were also consistent with results from J774A.1 phagocytosis assays as well (FIG. 6B).

The original ‘Self’ peptide inhibited phagocytosis, but the present data show that these peptides activate phagocytosis. Without wishing to be limited by any theory, these peptides are soluble whereas studies on the ‘Self’ peptide were on particles (i.e. solid). These peptides are small, which confer an advantage over large antibody antagonists and even polypeptide ectodomains in being able to potentially penetrate tissues into solid tumors. Furthermore, un-opsonized, anti—CD47 blocked RBCs were eaten by macrophages; whereas it should be noted that the peptides herein lack activating antibody Fc chains and potentially solely inhibit the SIRPα binding pocket.

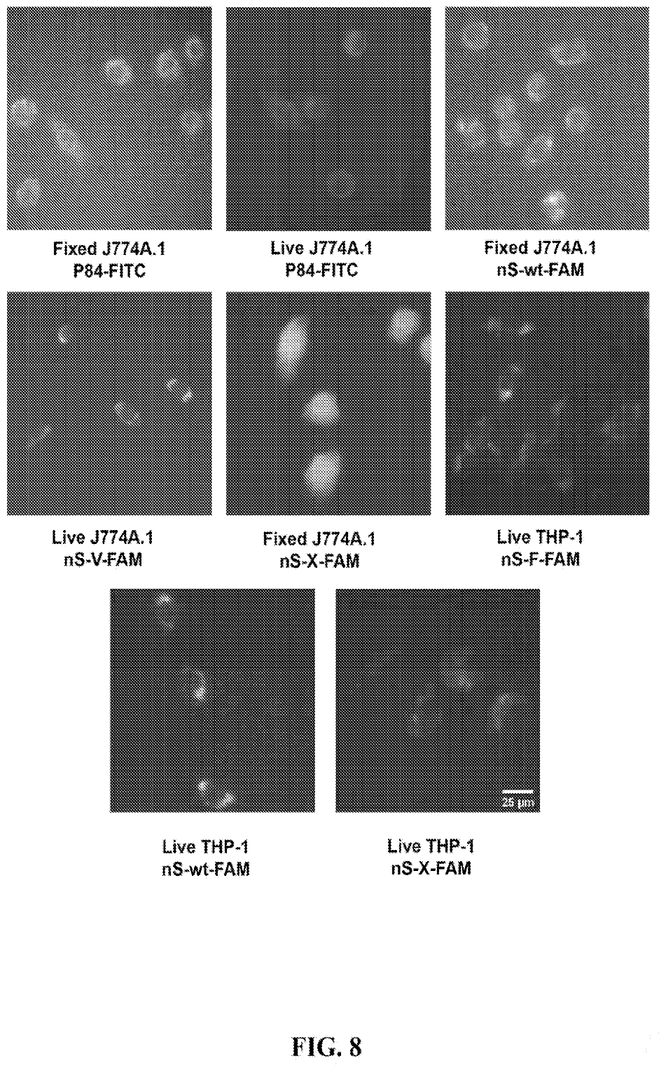

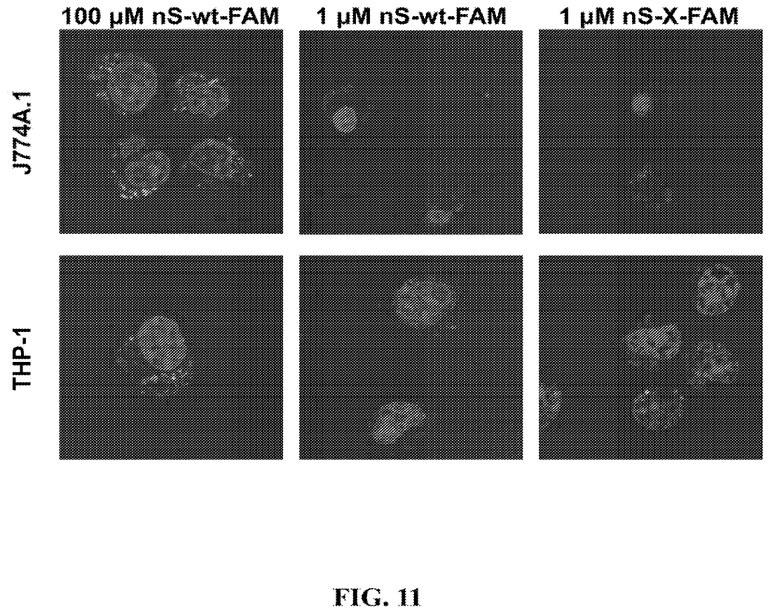

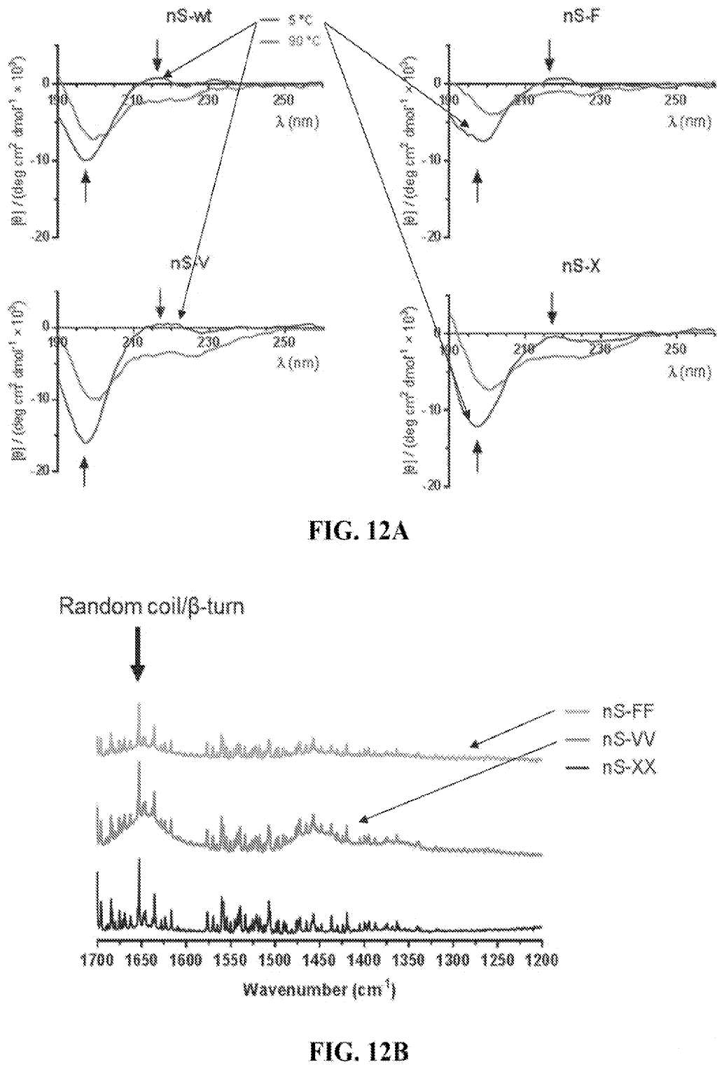

As shown herein, nS peptides bind to SIRPα on the surface of macrophages but are outcompeted by bivalent peptides. The main observation between binding of the peptides to live and fixed macrophages was the crescent shape formed when bound to live cells. While the intensity of nS-wt, nS-V and nS-F were similar and significantly greater than that of nS-X, the fact that the crescent shape was also observed when nS-X was bound indicated that some peptide is being internalized by the macrophages. Confocal slices (FIG. 11) showed that indeed some of the peptides are internalized giving the crescent shape observed in 2D microscopy (FIG. 8). Without wishing to be limited by any theory, the internalization occurs through pinocytosis, where extracellular fluid containing the fluorescent peptides is internalized by the macrophages. The peptides accumulate within the macrophages in a concentration dependent manner and are not secreted. The bright fluorescence of nS-V and nS-F is attributed to be cumulative signal from the binding and internalization of the peptides by the macrophages because in fixed cells the peptides appear to only be on the surface of the macrophages. Furthermore, nS-X was also internalized; however, fluorescence intensity was significantly less compared to the other peptides, suggesting intensity is only due to the internalization of the peptide and not a result of binding. It was demonstrated the higher affinity of the bivalent nS peptides for THP-1 macrophages by inhibiting the binding of fluorescent monovalent nS-F (FIG. 3A). This is consistent with observed phagocytosis results with nS-FF potency at 20 nM ([+, FF] in FIG. 2B). The tighter and higher avidity of binding can further explained by the CD spectra (FIGS. 4A-4B) of nS-FF and nS-VV.

As shown herein, nS peptides inhibit cis interactions between CD47 and SIRPα on macrophages. There have been reports suggesting that CD47 can interact with SIRPα in cis on the same. In certain embodiments, this may be an explanation to the basal level of SIRPαphosphorylation observed here. Phagocytosis is inhibited upon binding of these two receptors; however, increased phagocytosis is reported with the use of the present peptides. Through fluorescence imaging analysis, it was possible to confirm the decrease in phosphorylation levels after incubating isolated THP-1 macrophages with nS-F and nS-FF (FIG. 3D). This further supports the finding that these peptides indeed bind to SIRPα and inhibit CD47 binding and that multivalency increases such effects. The data confirm the trends observed in the phagocytosis assays ([+,F], [+,FF] in FIG. 2B). At 20 nM of nS-F, phosphorylation levels do not change with respect to control THP-1 macrophages consistent with the lower effect on phagocytosis and inhibiting mainly trans interactions. However, 20 nM of nS-FF suppressed passivating pTyr intensity consistent with potent activity on phagocytosis and inhibiting both cis and trans CD47 binding. The present work is the first example of SIRPα inhibitors confirming that the disruption of cis and trans CD47 binding enhances phagocytosis.

As shown herein, some nS peptides have some propensity to fold into hairpins suggesting an induced fit binding mechanism. It was investigated whether the nS peptides fold into a β-hairpin or any other secondary structure in solution or whether they bound SIRPα in an induced fit fashion. From CD analysis, it was inferred that nS-wt, nS-F and nS-V (FIG. 12A) have, to an extent, a β-hairpin fold with much more of a random coil conformation. This was further validated with Fourier Transform infrared (FT-IR) analysis (FIG. 12B). Although the structure of the nS peptides do not entirely exhibit β-turns, the biologically relevant effect observed with phagocytosing opRBCs suggests an induced fit mechanism. Without wishing to be limited by any theory, considering the peptides may have secondary structure at low temperatures (5° C.), it is unlikely that they conform to β-hairpin turns at 37 ° C., which is the temperature at which the phagocytosis and other key assays were executed.

### Compounds

The present disclosure provides a peptide (P1), or a salt or solvate thereof, comprising the amino acid sequence of SEQ ID NO:1:

Val Thr Glu Leu Xaa5 Arg Glu Gly (SEQ ID NO:1)

wherein Xaa5 is Thr or an amino acid (natural or synthetic) that is not Thr, such as for example an amino acid that is less polar than Thr.

In certain embodiments, the peptide P1, or a salt or solvate thereof, consists essentially of the amino acid sequence of SEQ ID NO:1.

In certain embodiments, the peptide P1, or a salt or solvate thereof, consists of the amino acid sequence of SEQ ID NO: 1.

In certain embodiments, Xaa5 is Thr. In certain embodiments, Xaa5 is Phe. In certain embodiments, Xaa5 is Val. In certain embodiments, Xaa5 is Ala. In certain embodiments, Xaa5 is Leu. In certain embodiments, Xaa5 is Ile. In certain embodiments, Xaa5 is Pro. In certain embodiments, Xaa5 is Met. In certain embodiments, Xaa5 is Ser.

In certain embodiments, Xaa5 is not Thr. In certain embodiments, Xaa5 is not Phe. In certain embodiments, Xaa5 is not Val. In certain embodiments, Xaa5 is not Ala. In certain embodiments, Xaa5 is not Leu. In certain embodiments, Xaa5 is not Ile. In certain embodiments, Xaa5 is not Pro. In certain embodiments, Xaa5 is not Met.

In certain embodiments, in the peptide P1 the N-terminus of the amino sequence of SEQ ID NO: 1 is directly coupled with the C-terminus of an amino acid or peptide (which itself may be part of a peptide or polypeptide) that is not Gly-Asn-Tyr-Thr-Cys-Glu, Asn-Tyr-Thr-Cys-Glu, Tyr-Thr-Cys-Glu, Thr-Cys-Glu, Cys-Glu, Thr-Glu, or Glu.

In certain embodiments, in the peptide P1 the C-terminus of the amino sequence of SEQ ID NO:1 is directly coupled with the N-terminus of an amino acid or peptide (which itself may be part of a peptide or polypeptide) that is not Glu-Thr-Ile-Ile-Glu, Glu-Thr-Ile-Ile, Glu-Thr-Ile, Glu-Thr, Glu-Cys, or Glu.

In certain embodiments, P1 comprises Cys^{a}—SEQ ID NO:1-Cys^{b}. In certain embodiments, Cys^{a} and Cys^{b} are bridged by a disulfide bond resulting in a cyclic peptide.

The present disclosure further provides a compound of Formula (I), or a salt or solvate thereof:

(BINDERᵢ- LINKERᵢ)ᵢCORE (I)

wherein:
each occurrence of BINDERᵢ is independently a P1 peptide as defined elsewhere herein;
each occurrence of LINKERᵢ is independently a linker;
CORE is a moiety independently covalently bound to each (BINDERi-LINKERi) through LINKERi;
‘i’ is an integer equal to or greater than 2.

In certain embodiments, ‘i’ is 2. In certain embodiments, ‘i’ is 3. In certain embodiments, ‘i’ is 4. In certain embodiments, ‘i’ is 5. In certain embodiments, ‘i’ is 6. In certain embodiments, ‘i’ is 7. In certain embodiments, ‘i’ is 8. In certain embodiments, ‘i’ is 9. In certain embodiments, ‘i’ is 10. In certain embodiments, ‘i’ is 11. In certain embodiments, ‘i’ is 12. In certain embodiments, ‘i’ is greater than 12.

In certain embodiments, ‘i’ is 2 and the compound, or a salt or solvate thereof, is:

(BINDER1-LINKER1)-CORE-(LINKER2-BINDER2) (I-1).

In certain embodiments, ‘i’ is 3 and the compound, or a salt or solvate thereof, is:

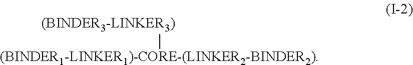

In certain embodiments, ‘i’ is 4 and the compound, or a salt or solvate thereof, is:

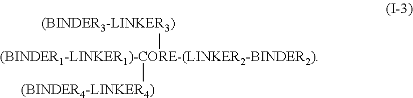

In certain embodiments, at least one BINDERᵢ comprises the amino acid sequence of SEQ ID NOs: 6-8.

In certain embodiments, each linker LINKERᵢ independently comprises a group of Formula (II):

*-(CHR′)ₘ₁-X¹-(CHR′-CHR′-X²)ₘ₂-(CHR′)ₘ₃-X³- (II)

wherein:
* indicates the bond between the LINKERᵢ and the BINDERi;
m1, m2, and m3 are independently an integer ranging from 0-5, 0-10, 0-20, 0-30, 0-40, 0-50, 0-60, 0-70, 0-80, 0-90, or 0-100;
each occurrence of X¹ and X² are independently selected from the group consisting of absent (a bond), O, and N(R′);

X³ forms a covalent bond to the CORE and is selected from the group consisting of absent (a bond), C(═O), O, S, and N(R′);

each occurrence of R′is independently selected from the group consisting of hydrogen, optionally substituted C₁-C₆ alkyl, optionally substituted C₃-C₈ cycloalkyl, and optionally substituted C₃-C₈ cycloheteroalkyl.

In certain embodiments, each LINKERᵢ is independently about 5 Å, 6 Å, 7 Å, 8 Å, 9 Å, 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, 25 Å, or greater than 25 Å in length.

In certain embodiments, each LINKERᵢ independently has a linear length of about 5, 6, 7, 8, 9, 10, 11, 12, 13, 14, 15, 16, 17, 18, 19, 20, 21, 22, 23, 24, 25, or greater than 25 atoms.

In certain embodiments, the CORE is a chemical moiety comprising ‘i’ groups, wherein each of these groups is covalently linked to an individual LINKERi. In certain embodiments, each group is independently a carboxylic acid, a primary or secondary amine, a hydroxyl group, a thiol group, and/or an alkene group. For example, when the group is a carboxylic acid, it can be covalently linked to a primary or secondary amine on LINKERᵢ through an amide bond. For example, when the group is a primary or secondary amine, it can be covalently linked to a carboxylic acid on LINKERᵢ through an amide bond. For example, when the group is a hydroxyl group, it can be covalently linked to a carboxylic acid on LINKERᵢ through an ester bond or can be alkylated with LINKERᵢ. For example, when the group is a thiol group, it can be covalently linked to a carboxylic acid on LINKERᵢ through a disulfide bond or can be alkylated with LINKERᵢ. For example, when the group is an alkene group, it can be covalently linked to a double bond on LINKERᵢ through an olefin metathesis reaction or to a diene group on LINKERᵢ through a Diels-Alder reaction.

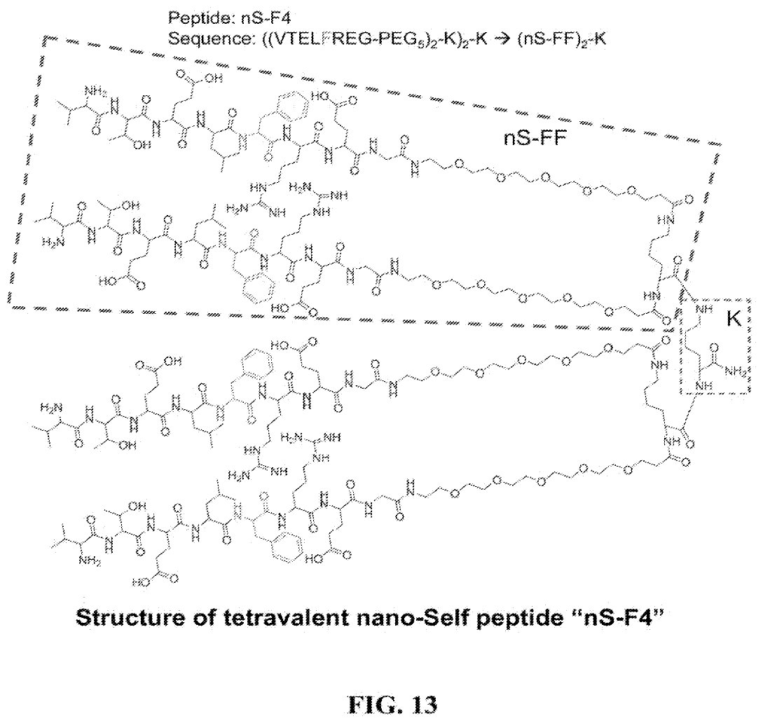

In certain embodiments, the CORE is an amino acid or (poly)peptide, wherein any of the amino acids of the CORE can be a naturally occurring or synthetic amino acid. In certain embodiments, the CORE is an amino acid or peptide comprising at least one of Asp, Glu, Lys, Arg, Ser, Thr, Orn, and Cys. In certain embodiments, the CORE can a single amino acid (such as Lys or Orn; FIG. 13). In other embodiments, the CORE can be a dipeptide. In yet other embodiments, the CORE can be a tripeptide. In yet other embodiments, the CORE can be a tetrapeptide. In yet other embodiments, the CORE can be a pentapeptide. In yet other embodiments, the CORE can be a hexapeptide. In yet other embodiments, the CORE can be a peptide comprising 7 or more amino acids. In yet other embodiments, the peptide in the CORE can be chemically modified. For example, one or more of the internal amide groups can be alkylated (for example, methylated). For example, the N-terminus of the peptide can be acylated (for example, formylated or acetylated) and/or mono- or di-alkylated (for example, mono- or di-methylated). For example, the C-terminus of the peptide can be amidated (for example, as a primary, secondary, or tertiary amide) or esterified (for example, as a methyl or ethyl ester).

In certain embodiments, a LINKERᵢ can be linked through an amide bond to the C-1 (head) carboxylic acid of the amino acid. In certain embodiments, a LINKERᵢ can be linked through an amide bond to the C-2 (alpha) amino group of the amino acid. In certain embodiments, a LINKERᵢ can be linked through an amide bond to any additional carboxylic acid of the amino acid (such as in the case of aspartic acid and/or glutamic acid) . In certain embodiments, a LINKERᵢ can be linked through an amide bond to any additional amino group of the amino acid (such as in the case of lysine and/or arginine) . In certain embodiments, a LINKERᵢ can be linked through a covalent bond to a hydroxyl group of the amino acid (such as in the case of serine and/or threonine). In certain embodiments, a LINKERᵢ can be linked through a covalent bond to a thiol group of the amino acid (such as in the case of cysteine).

Compounds of Formula (I) or compounds otherwise described herein can be prepared by the general schemes described herein, using the synthetic method known by those skilled in the art. The following examples illustrate non-limiting embodiments of the invention.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/ or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the invention may exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the invention are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc.

Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base- protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

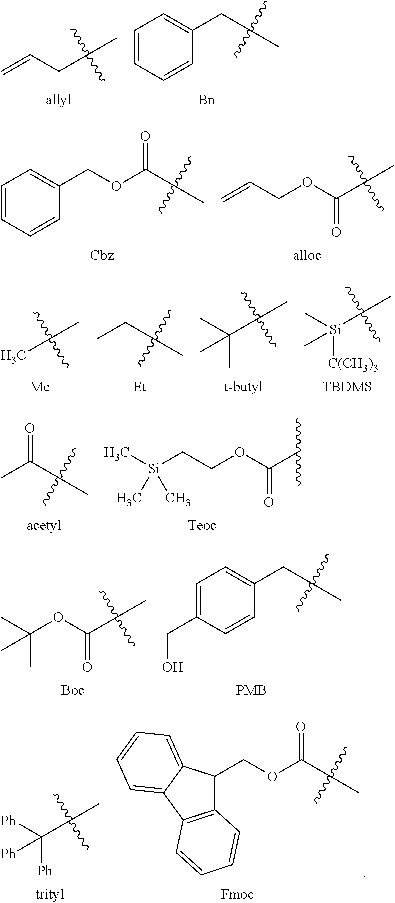

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Compositions

The invention includes a pharmaceutical composition comprising at least one compound of the invention and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration. In various embodiments, a pharmaceutical composition includes the compound of Formula (I), or salts and solvates thereof, and at least one pharmaceutically acceptable excipient.

### Administration/Dosage/Formulations

The regimen of administration may affect what constitutes an effective amount. The therapeutic formulations may be administered to the subject either prior to or after the onset of a disease or disorder contemplated herein. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated herein. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated herein in a patient.

In certain embodiments, the compositions of the invention are formulated using one or more pharmaceutically acceptable excipients or carriers. In certain embodiments, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physician taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated herein in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of a disease or disorder contemplated herein in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. The data obtained from cell culture assays and animal studies are optionally used in formulating a range of dosage for use in human. The dosage of such compounds lies preferably within a range of circulating concentrations that include the ED50 with minimal toxicity. The dosage optionally varies within this range depending upon the dosage form employed and the route of administration utilized. Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

The materials and methods employed in these experiments are now described.

### Materials & Methods

Standard peptide synthesis. All peptides in this study were synthesized on a Rink Amide MBHA Resin (loading density: 0.33 mmol/g; Novabiochem) on a 100 μmol scale at room temperature (RT) using 9-fluorenylmethoxycarbonyl (Fmoc) chemistry. The resin was transferred to a solid phase peptide synthesis vessel and swelled in N,N-dimethylformamide (DMF; Sigma) for 30 minutes with stirring. Deprotection of the Fmoc group was achieved by using 1 mL of 1% w/v 1-hydroxybenzotriazole (HOBT; EMD Millipore) and 2% v/v 1,8-diazabicyclo[5.4.0]undec-7-ene (DBU; Acros Organics) in DMF and left to stir for 1 minute (repeated three times). Lastly, resin was then washed thoroughly with DMF. Coupling solutions contained 3 equivalents of Fmoc-amino acids (Chem-Impex or Oakwood Chemicals), 2.8 equivalents of 1-[bis(dimethylamino)methylene]-1H-1,2,3-triazolo[4,5-b]pyridinium 3-oxid hexafluorophosphate (HATU; Oakwood Chemicals), and 6 equivalents of N,N-diisopropylethylamine (DIEA; Sigma) - relative to resin - dissolved in minimal amount of DMF to cover resin (1-1.3 mL) and were activated for 5 minutes at RT prior to addition to resin. Coupling reactions were left to proceed for 1 hour. Following each coupling reaction, the resin was drained, washed thoroughly with DMF, deprotected as described elsewhere herein and washed thoroughly with DMF again.

SIRPα Expression and Purification. Soluble human SIRPα fused with glutathione s-transferase (SIRPα-GST) was expressed and purified as previously published (Tsai, et al., 2008, J. Cell Biol. 180:989-1003).

Bivalent peptide synthesis. Bivalent peptides were prepared by coupling 3 equivalents of Fmoc-Lys(Fmoc)—OH directly on resin and deprotecting the Fmoc groups following the same procedure mentioned elsewhere herein. The coupling solutions of the polyethylene glycol (PEG) acids contained 5 equivalents of Fmoc—NH-PEG₅-CH₂CH₂COOH (PurePEG LLC, San Diego CA), 4.5 equivalents of HATU and 10 equivalents of DIEA. The coupling reactions were left to proceed for 3 hours. Every subsequent amino acid coupling was done using 6 equivalents Fmoc-amino acid, 5 equivalents HATU and 10 equivalents DIEA.

Tetravalent peptide synthesis. Tetravalent peptides were prepared by coupling 3 equivalents of Fmoc-Lys(Fmoc)—OH directly on resin and deprotecting the Fmoc groups following the same procedure mentioned elsewhere herein. A second lysine coupling was done by adding 6 equivalents of Fmoc-Lys(Fmoc)—OH, 5.5 equivalents HATU, and 10 equivalents of DIEA. The coupling solutions of the polyethylene glycol (PEG) acids contained 12 equivalents of Fmoc—NH-PEG₅-CH₂CH₂COOH, 11 equivalents of HATU and 20 equivalents of DIEA. The coupling reactions were left to proceed for 3 hours. Every subsequent amino acid coupling was done using 12 equivalents Fmoc-amino acid, 11 equivalents HATU and 20 equivalents DIEA.

Generation of cyclic peptides. Cyclic peptides were prepared by coupling Fmoc-Cyc(trt)-OH as the first and last amino acids. Deprotection of the Fmoc groups was achieved following the same procedure mentioned above. After peptide cleavage and ether precipitation, the peptide pellet was exposed to air to allow for disulfide oxidation.

5(6)-Carboxyfluorescein (FAM) coupling. All fluorescently labeled peptides were prepared by coupling Boc-Lys(Fmoc)—OH at the N-terminus and deprotection of the Fmoc-protected γ-amine of Lys. FAM (Chem-Impex) was prepared by dissolving 2 equivalents in DMF with 2 equivalents of HATU and added to the resin after activation for 5 minutes at RT. 6 equivalents of DIEA were added dropwise to the stirring solution in order to maintain a homogenous solution. The reaction was left to proceed overnight in the dark.

Peptide cleavage. Following the final deprotection of the last Fmoc group (except for fluorescent peptides where the last amino acid contains acid labile Boc protecting group), the resin was washed with DMF twice and then twice more with dichloromethane (DCM; Sigma). A 5 mL cleavage cocktail containing 95% trifluoracetic acid (TFA; Acros Organics), 2.5% H₂O and 2.5% triisopropylsilane (TIPS; Oakwood) was added to the reaction vessel and left to stir for 4 hours. 45 mL of cold diethyl ether (Sigma) was then added to the cleavage solution precipitating the peptide. To make sure all peptide precipitated, the ether layer was evaporated by air until ˜10 mL of solution was left; thereafter, an additional 40 mL of cold ether was added. The peptide was collected by centrifugation, resuspended in cold ether and collected by centrifugation again (repeated three times). Depending on the solubility of the peptide, the ether 10 washed pellet was dissolved in a mixture of 10-40% acetonitrile (ACN; Sigma) in water.

Purification and characterization. All peptides were purified using preparative reversed-phase high-performance liquid chromatography (HPLC) on an Agilent 1260 Infinity II system using a Phenomenex Luna Omega 5 μm PS C18 100 Å LC column. Varying gradients of ACN and 0.1% TFA in H₂O were used to separate the respective peptides. Purity of each peptide was 15 checked using an analytical Agilent 1260 Infinity II system using a Phenomenex Luna Omega 5 μm PS C18 100 Å LC column. Mass spectrometry was performed using a Bruker matrix-assisted laser desorption ionization—time of flight (MALDI-TOF) Ultraflex III mass spectrometer and α-Cyano-4-hydroxycinnamic acid (CHCA; Sigma) as the matrix. Peptides were lyophilized using a Labconco FreeZone Plus 12 Liter Cascade Console Freeze Dry system.

### nano—Self Peptides Binding to soluble SIRPα

Peptide biotinylation: NHS-biotin (Thermo Fischer) was mixed with nS-wt and nS-V, respectively, following the manufacturer's protocol. The biotinylated peptides were dialyzed against PBS.

Streptavidin-beads binding: Streptavidin-coated polystyrene beads of 2.1 μm radius (Spherotech) were washed and blocked 3x in PBS plus 0.4% BSA. nS-wt-biotin or nS-V-biotin were incubated with the beads for 1 hour at room temperature with shaking. Beads were washed with PBS, then incubated with recombinant SIRPα for 1 hour in ice. Streptavidin beads were labeled with rabbit anti-streptavidin-FITC (Invitrogen) and SIRPα was stained with mouse anti-SIRPα-allophycocyanin (anti-SIRPα-APC; Invitrogen).

Affinity binding assay. The same binding protocol mentioned elsewhere herein was used. Increasing concentrations (10 nM to 1 mM) of FAM-labeled peptides were incubated with THP-1 macrophages before or after fixation. For J774A. 1 macrophages, affinity binding assay was only done on live cells. Fluorescence imaging and analysis were done as above.

Opsonin Titration. The same phagocytosis protocol mentioned elsewhere herein was used. Increasing concentrations (33 nM-1.33 μM) of RBC opsonin were incubated with the RBCs. RBC CD47 was blocked in all conditions with 5 μg/mL anti-CD47. Fluorescence imaging and analysis were done as above.

UV-Vis, circular dichroism (CD) and Fourier Transform infrared (FT-IR) measurements. UV-Vis absorption spectrophotometry was performed using a Jasco V-650 Spectrophotometer and 1 cm path length quartz cells. Lyophilized peptide was dissolved in 100 μL of phosphate buffered saline pH 7.4 (PBS; Thermo Fischer) and concentration of each peptide was determined by measuring the absorbance at 205 nm and using a calculated extinction coefficient for each peptide due to the lack of aromatic residues in the peptides. For fluorescein labeled peptides, the lyophilized solid was dissolved in 20 μL of dimethyl sulfoxide (DMSO; Sigma) then diluted to 100 μL with PBS. Peptide concentration was determined by measuring the absorbance at 495 nm.

CD experiments were performed using a Jasco J-1500 Circular Dichroism Spectrometer and 1 mm quartz cuvettes. 100 μM samples were prepared for each peptide in sodium phosphate buffer pH 7 and ellipticity was measured from 190 nm to 260 nm at 5 ° C.and 95 ° C., respectively.

FT-IR measurements were collected using a Jasco FT/IR-6800 FT-IR spectrometer. Peptide samples were solvent swapped into in deuterated water and deuterated hydrochloric acid. 5 μL droplets of peptide samples were measured at room temperature and absorbance was recorded from 1200-1700 cm⁻¹.

Cell culture. All cells were purchased from American Type Culture Collection (ATCC). Human derived THP-1 monocytes and mouse J774A. 1 macrophages were cultured in RPMI 1640 media (Gibco). Human lung carcinoma A549 cells were cultured in Ham's F12 nutrient mixture (Gibco) and monkey kidney COS-1 cells were cultured in DMEM high-glucose media (Gibco). All media were supplemented with 10% fetal bovine serum (FBS; Sigma) and 1% penicillin/streptomycin (Sigma). Detachment of adherent cells for passaging was done by using 0.05% (for A549) or 0.25% (for COS-1) Trypsin/0.5mM EDTA (Invitrogen). J774A.1 macrophages were grown either as suspension or adherent cultures. To passage adherent J774A.1 macrophages, the cells were gently scraped with a cell scraper (Corning). THP-1 monocytes were cultured in suspension. Differentiation of THP-1 monocytes to macrophages was achieved by addition of 100 ng/mL phorbol myristate acetate (PMA; Sigma) in media for 2 days (unless stated otherwise) and confirmed by attachment of the macrophages to the bottom of the tissue culture plates.

B16F10 SIRPα knockout cells were prepared as previously described. Briefly, B16F10 parental cells were transduced with lentivirus for expression of Cas9 and single guide RNA against SIRPα (5′-TAATTCTAAGGTCATCTGCG-3′, designed using the Broad Institute's CRISPick, manufactured by Integrated DNA Technologies, and integrated into plasmid by using BsmBI restriction digest). Knockout was confirmed by flow cytometry by using anti-mouse SIRPα (P84, Biolegend) with secondary donkey anti-mouse AlexaFluor 647 (Thermo Fisher).

The plasmids LentiV-Cas9_puro and Lenti_sgRNA_EFS_GFP plasmids (Addgene #108100 and 65656, respectively) were gifts from Christopher Vakoc. Lentivirus for delivery was done using HEK 293T cells by co-transfecting them with the desired transfer plamids, pVSVg, and psPAX² at a 2:2:1 ratio (by mass) using MIrus TransIT (MIR6603) transfection reagent. Viral supernatant was collected 48 h after transfection, added to target cells at a 1:1 volumetric ratio with regular cell culture media. Successfully transduced cells were selected using puromycin at 2 μg/ml.

SIRPα staining and confocal imaging. THP-1 monocytes were PMA-differentiated in RPMI 1640 for 24 hours. J774A.1, A549, and COS-1 cells were plated for 24 hours in media, respectively. Cells were washed with respective media prior to addition of fluorescent peptides. The peptides were left to incubate for 1 hour at 37 ° C., 5% CO₂ and 95% humidity. For fixed cell staining, cells were incubated with 4% formaldehyde (Pierce) for 15 minutes prior to the addition of fluorescent peptides whereas for live cell staining, fixation was done after incubation with the FAM-labeled peptides. Nuclei were stained with 1 μg/mL Hoechst 33342 (Invitrogen). Cells were the washed PBS three times before analysis. Fluorescence imaging of fixed cells was performed using an Olympus IX71 with a digital EMCCD camera (Cascade 512B) and a 40×/0.6 NA objective. For live cell (without fixation) confocal imaging, cells were washed with respective media three times after nuclei staining instead of PBS. Confocal imaging was done using a Leica TCS SP8 system with 63×/1.4 NA oil-immersion objective. Quantification was done with ImageJ (NIH).

Peptide inhibition assay. THP-1 monocytes were PMA differentiated in RPMI for 48 hours. Macrophages were washed with RPMI three times then incubated with 50 μM, 1 μM, or 20 nM nS-FF or nS-VV for 1 hour at 37° C., 5% CO₂ and 95% humidity. Excess peptide was washed with PBS then 100 μM of either FAM labeled nS-wt or nS-F were added to the bivalent-peptide blocked macrophages for 1 hour as described elsewhere herein. To measure background internalization of the FAM-labeled peptides, nS-X-FAM was used. To determine the efficacy of the bivalent peptides to inhibit SIRPα binding, nS-X was used. Excess peptides were washed off with PBS and cells were fixed with 4% formaldehyde for 15 minutes at RT, washed with PBS, stained with 1 μg/mL Hoechst 33342, and then washed with PBS again. Fluorescence imaging was performed as described elsewhere herein and quantification was done using ImageJ.

In vitro phagocytosis assay. Fresh human RBCs were washed twice with 50 mM EDTA (Thermo Fischer) in PBS (Gibco) then twice with 5% FBS in PBS. RBCs were opsonized with 20 μg/mL rabbit anti-human RBC antibody (Rockland) in 5% FBS for 1 hour at RT with shaking. For CD47 blocked RBCs, 5 μg/mL of mouse anti-human CD47 (B6H12; BD Biosciences) were added. Thereafter, RBCs were washed with PBS three times and stained with PKH26 dye (1:800 dilution in PBS; Sigma) for 1 hour at RT with shaking in the dark. RBCs were washed and resuspended in PBS.

THP-1 monocytes were PMA differentiated in RPMI for 48 hours. Macrophages were then washed with RPMI media three times. For SIRPα blocked THP-1 macrophages, 20 nM, 1 μM or 50 μM of the nano—Self peptides were incubated with the THP-1 macrophages for 1 hour at 37 ° C., 5% CO₂ and 95% humidity. Those THP-Is were then washed with RPMI three times.

J774A.1 macrophages were plated for 24 hours in RPMI. For SIRPα blocked J774A.1 macrophages, the same peptide blocking procedure as described elsewhere herein was used with the addition of a positive control using 5 μg/mL rat anti-mouse SIRPα (P84; BD Biosciences). Opsonized RBCs were added to macrophages at a ratio of 10:1 for 1 hour at 37° C., 5%

CO₂ and 95% humidity. Macrophages were then washed with RPMI three times. Adherent and uninternalized RBCs were lysed with water for 30 seconds followed by immediate replacement with RPMI media. In order to distinguish remaining adherent RBCs from internalized RBCs, opsonized RBCs were stained with AlexaFluor 647 donkey anti-rabbit (binds to rabbit polyclonal opsonin on RBC; Invitrogen) IgG (1:1000) while unopsonized, CD47 blocked RBCs were stained with AlexaFluor 647 donkey anti-mouse (binds to mouse monoclonal anti-CD47 on RBCs; Invitrogen) IgG (1:1000) for 30 minutes. After washing, macrophages were fixed with 4% formaldehyde for 15 minutes at RT, washed with PBS, stained with 1 μg/mL Hoechst 33342, and then washed with PBS again. Fluorescence imaging was performed as described elsewhere herein and quantification was done using ImageJ. At least 200 cells were analyzed and experiments were repeated at least three times.

B16F10 cells: Bone marrow cells were isolated from femurs and tibias of healthy C57BL6/J mice (The Jackson Laboratory) and cultured in 10-cm petri dishes containing IMDM (Gibco) supplemented with 10% v/v FBS, 1% v/v penicillin/streptomycin, and 1:10,000 macrophage colony stimulating factor cytokine (M-CSF; Biolegend) for 7 days at 37° C., 5% CO₂ and 95% humidity. All animal experiments were performed according to protocols approved by the University of Pennsylvania's IACUC (protocol #805977 and #804455). The resulting mouse bone marrow derived macrophages (BMDMs) were washed with PBS containing Ca2⁺/Mg2⁺ (Gibco) then detached with 0.25% trypsin-EDTA (Gibco) and re-plated in 6-well tissue culture plates using IMDM+M-CSF for 24 hours.

Mouse melanoma B16F10 cells were washed with PBS containing Ca2-/Mg2- and then trypsinized with 0.05% trypsin-EDTA. After washing twice with PBS, the cells were labeled with 1:10,000 CFDA—SE (Thermo Fisher) in PBS for 10 minutes. B16F10 cells were opsonized with 10 μg/mL anti-TYRP1 (TA99; Bio X Cell) in PBS for 2 hours at 4° C.with occasional inverting. For CD47 blocked B16F10 cells, 5 μg/mL of anti-mouse CD47 (B6H12; BD Biosciences) were added. During the incubation time, the BMDMs were washed with media then stained with 1:2,000 CellTracker Deep Red (Thermo Fisher) in PBS with Ca2⁺/Mg2⁺ for 10 min. After washing, 50 μM of the nano—Self peptides were incubated with the BMDMs for 1 hour at 37° C., 5% CO₂ and 95% humidity. Finally, BMDMs were washed twice with PBS and then twice with serum-free IMDM.

Without removing the supernatant, the opsonized melanoma cells were added to the labeled BMDMs at a ratio of 2:1 for 2 hours at 37° C., 5% CO₂ and 95% humidity. During the last 15 minutes of the assay, the cells were Hoechst stained. The wells were washed with serum free IMDM then fixed with 4% formaldehyde for 15 minutes at RT, and PBS washed. Fluorescence imaging was performed using an Olympus IX71 with a digital EMCCD camera (Cascade 512B) and a 40×/0.6 NA objective. Quantification was done with ImageJ (NIH). At least 200 cells were analyzed and two-tailed student's t-test was used to determine statistical significance.

Phosphotyrosine (pTyr) staining. THP-1 monocytes were PMA differentiated for 48 hours. Macrophages were washed with RPMI three times then incubated with 50 μM and 20 nM of either nS-F or nS-FF for 1 hour at 37° C., 5% CO₂ and 95% humidity. The same conditions were replicated with the addition of 5 μg/mL anti-CD47. Excess peptide was washed with PBS then macrophages were fixed. Permeabilization of the macrophages was achieved with 0.5% Triton-X for 30 minutes. After washing with PBS, the macrophages were incubated with 1:100 mouse anti-pTyr (Santa Cruz Biotechnology) for 1 hour at room temperature with shaking. Macrophages were washed with PBS then stained with AlexaFluor 488 donkey anti-mouse (1:1000; Invitrogen) and 1 μg/mL Hoechst 33342 for 1 hour with shaking then washed again. Fluorescence imaging and quantification was performed as described elsewhere herein.

CD47-Fc Inhibition Assay. THP-1 monocytes were PMA differentiated in RPMI for 48 h. The macrophages were washed with RPMI media and then incubated with 1 μM or 50 μM of nano- Self peptides for 1 h at 37° C., 5% CO2, and 95% humidity. After washing, the macrophages were incubated with Human Trustain FcX Fc receptor blocking solution (Biolegend) for 10 min at RT and then incubated with 2 μg/mL CD47-Fc fusion protein (ACRO Biosystems) for 1 h at 37° C., 5% CO2, and 95% humidity. As a negative control, B6H12 was pre-mixed with CD47-Fc at 4° C. on a rotator for 1 h. The macrophages were then washed and incubated with 0.5 μg/mL goat anti-human IgG Fc DyLight 488 (Thermo Fischer) for 1 h at 37° C., 5% CO2, and 95% humidity. Finally, the macrophages were washed, fixed, Hoechst 33342 stained, and imaged as described above.

GEO Microarray Analysis. Data from the GEO database were used to obtain gene expression data for key genes associated with macrophage identity. The cell types included in this analysis were human HEK 293T (GEO accession GSE28715), human PMA-differentiated THP-1 macrophages (GEO accession GDS4258), primary mouse macrophages (GEO accession GDS2454), and human K562 erythroleukemia (GEO accession GSE16774 and GSE8832).

Flow Cytometry. Fresh human RBCs were washed twice with 50 mM EDTA and then twice with 5% w/v bovine serum albumin (BSA; Sigma) in PBS. K562 cells were collected and washed twice with 5% BSA. RBCs and K562 cells were blocked with 5% BSA for 1 h at RT on a rotator. Saturating amounts of AlexaFluor 647 mouse anti-human CD47 (B6H12; BD Biosciences) were added to both RBCs and K562 cells and incubated at RT on a rotator for 1 h. Cells were washed three times with 5% BSA. Flow cytometry was performed on a BD LSRII (Benton Dickinson) at the Penn Cytomics and Cell Sorting Resources Laboratory and analyzed with FCS Express 7 software (De Novo Software).

TABLE 1
Nano-Self peptides used in the invention.

| SEQ ID NO: | Name. | Sequence |
|---|---|---|
| 2 | nS-wt | VTELTREG |
| 3 | nS-F | VTELFREG |
| 4 | nS-V | VTELVREG |
| 5 | nS-X | LETVEGTR |
| 6 | nS-Cyc | CVELTREGC |
| 7 | nS-F-Cyc | CVTELFREGC |
| 8 | nS-X-Cyc | CLETVEGTRC |
| 9 | nS-FF | (VTELFREG-PEG₅)₂-K |
| 10 | nS-VV | (VTELVREG-PEG₅)₂-K |
| 11 | nS-XX | (LETVEGTR-PEG₅)₂-K |
| 12 | nS-F4 | ((VTELFREG-PEG₅)₂-K)₂-K |

The results of the experiments are now described.

### Example 1: nS Peptides Design, Synthesis, and Characterization

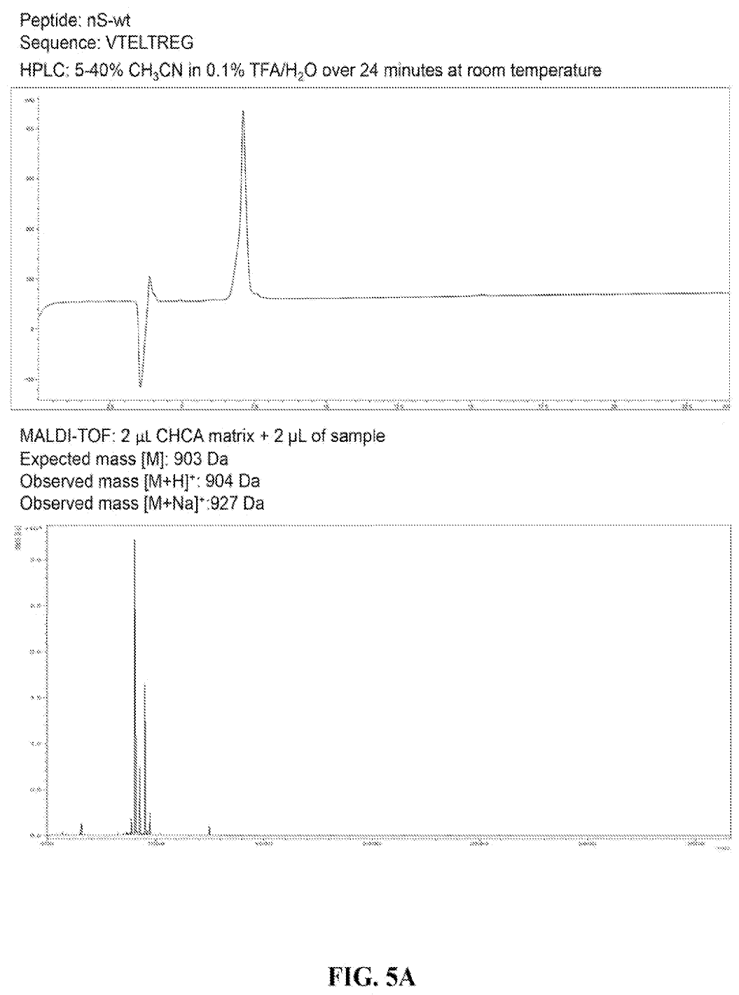

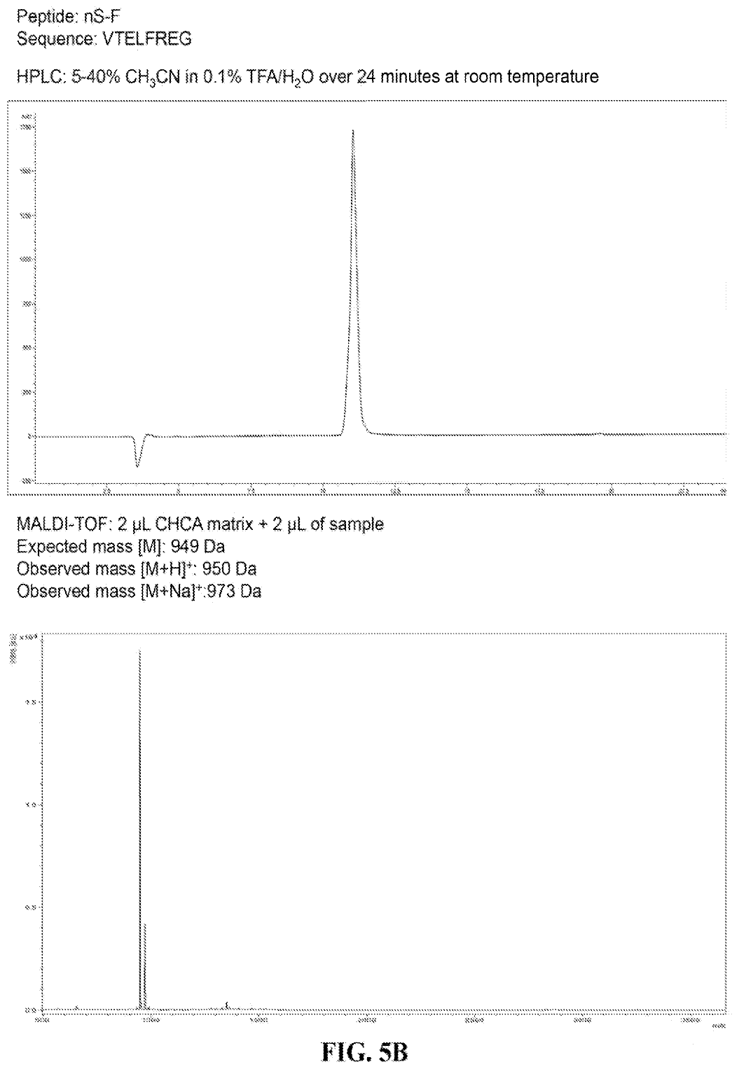

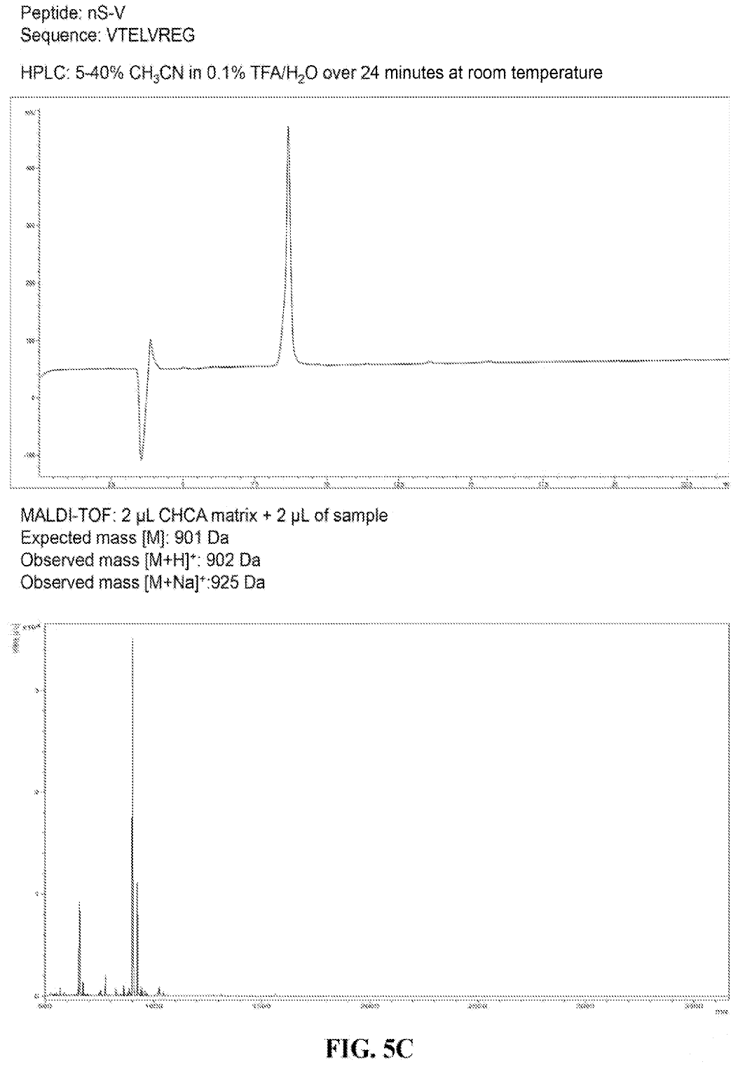

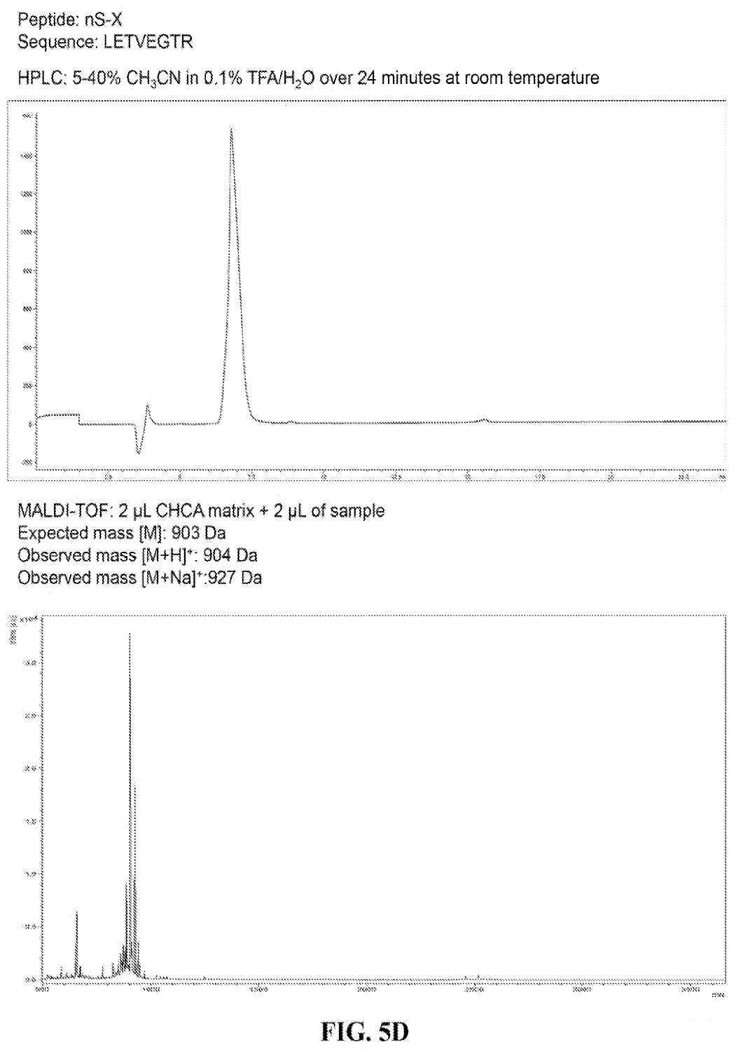

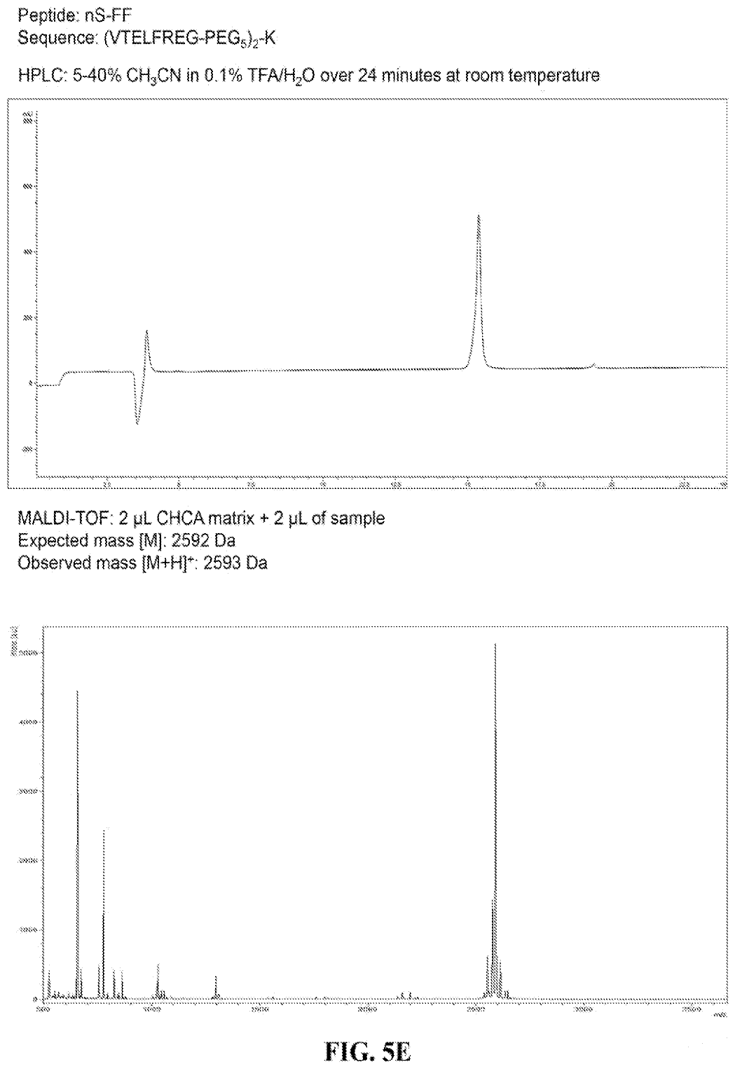

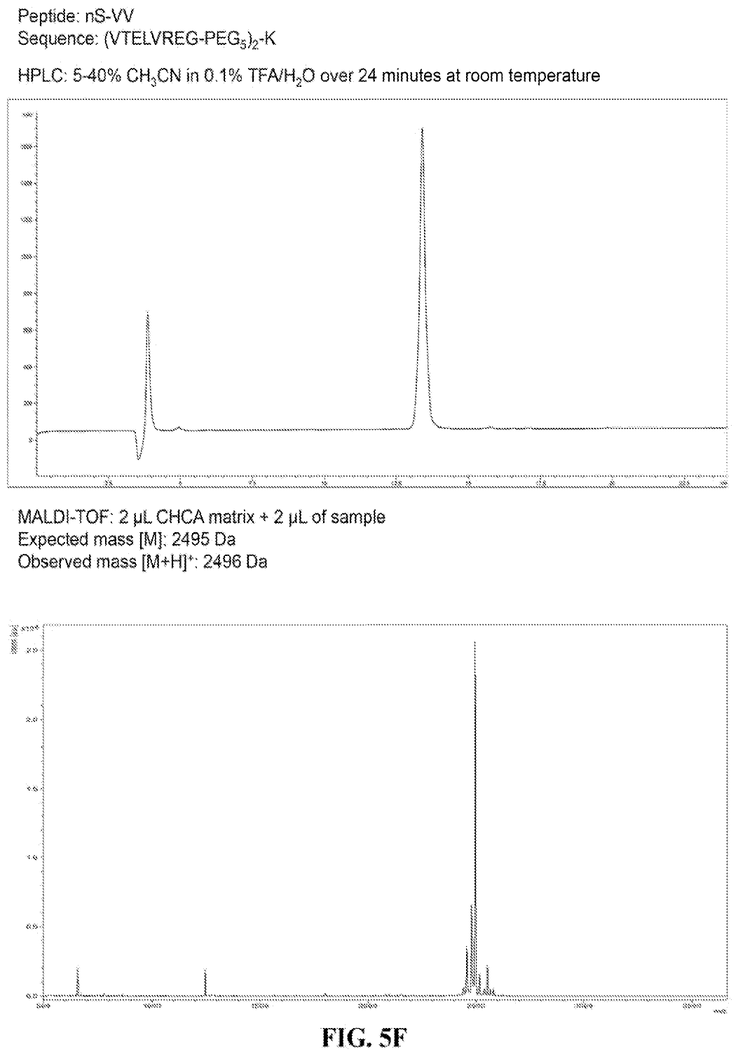

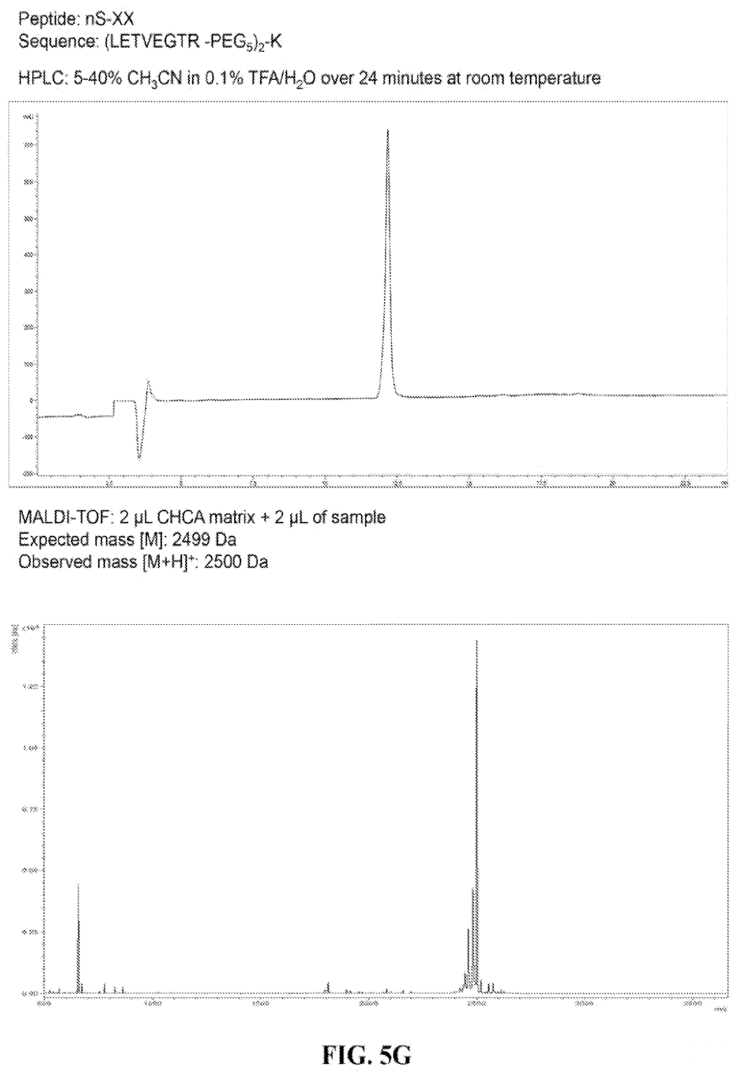

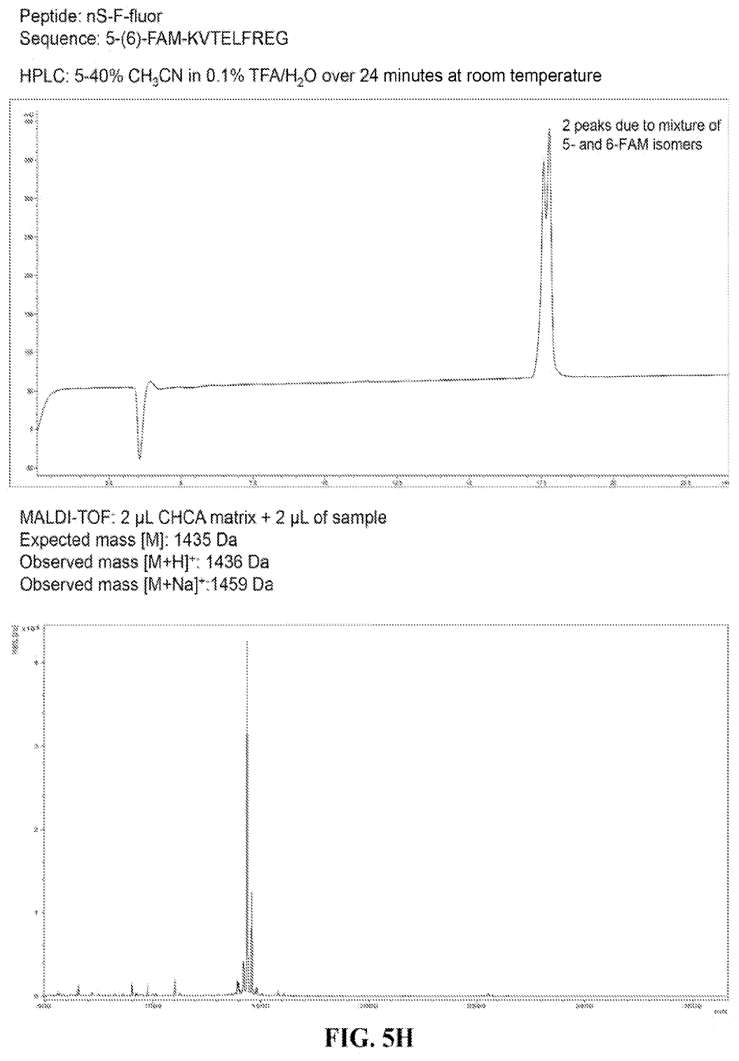

CD47′s binding site has a central T adjacent to a hydrophobic L that bury together in the main hydrophobic pocket of SIRPα. In certain non-limiting embodiments, mutation of the polar T to hydrophobic F or V introduces more favorable contacts and thereby increases the binding of nS peptides (FIG. 1, Table 1). SIRPα is also mobile - as it diffuses into the phagocytic synapse -and likely a homodimer. In certain non-limiting embodiments, a multivalent nS design binds with higher avidity to macrophages. Furthermore, modeling from crystal structures suggested a distance between binding sites on SIRPα homodimers of ˜25 Å, which led to the development of bivalent nS peptides possessing two flexible linkers of PEGs (˜15 Å each) attached to two amines of a central lysine. All nS peptides were synthesized on a Rink amide resin yielding C-terminal amide functional groups to minimize charge on the peptides. Analytical HPLC followed by MALDI-TOF mass spectrometry characterization indicated the high purity of all nS peptides (FIG. 5). Given that three of the eight residues in the nS peptides are charged at neutral pH, it is understandable that all peptides are soluble (up to at least 50 μM).

### Example 2: nS Peptides Increase Human Macrophage Eating of Opsonized Human Red Blood Cells

Solutions of nS peptides were added to cultures of adherent human THP-1 macrophages (FIG. 2A) in order to study the effects on phagocytosis of human red blood cells (RBCs). RBCs serve as a simple ‘Self’ cell and are also relevant to the anemia caused by infusion of anti-CD47 in the clinic and human erythroleukemia K562 cells which serve as a blood cancer cell model relevant to the liquid tumors that show some efficacy when treated with opsonized IgG and anti-CD47. Anti-CD47 antibody bound to the RBC (at 33 nM) has minimal effect on baseline engulfment (FIG. 2D), whereas anti-RBC provides a strong pro-phagocytic stimulus (referred to as opsonized), with ˜20% of macrophages engulfing at least one opRBC by the end of the 1 hr assay. Note that the concentration of anti-RBC opsonin used in the assay was enough to achieve half-max activity (FIG. 2E). Combining anti-CD47 with anti-RBC further stimulates phagocytosis, with ˜30% of macrophages eating because CD47 is blocked on the opRBCs ([+,+] in FIG. 2D). Mouse-derived J774A.1 macrophages show the same trends ([+,+,−] in FIG. 6B). Surprisingly, the bivalent nS-FF peptide at 20 nM was even more potent than anti-CD47 bound to opRBCs, with phagocytic macrophages increasing to ˜40% ([+,FF] in FIG. 2D).

The ˜10% increase in phagocytic macrophages with ns-FF peptide relative to anti-CD47 on opRBC matches the ˜10% increase recently measured upon disruption of cis interactions between CD47 and SIRPα on the same THP-1 macrophage (FIG. 2C-left). Depleting macrophage CD47 or adding anti-CD47 to macrophages indeed removes a passivating ‘Self’ signal from the cis interaction and thereby increases eating of a target. In certain non-limiting embodiments, the nS-FF peptide, which was designed to bind SIRPα, inhibits both its cis and trans interactions with CD47 (FIG. 2C-right). Such inhibition is nearly saturated based on higher concentration of nS-FF causing only modest increases in phagocytosis (˜5% for 1 μM, 50 M; FIG. 2E). In comparison, monovalent nS-F requires these higher concentrations to maximize engulfment ([+,F] in FIG. 2D), and shows only modest activity at 20 nM.

The nS-VV and nS-V peptides have activity profiles in phagocytosis assays that are similar to the monovalent nS-F (FIG. 2C). Without wishing to be limited by any theory, this could reflect the less hydrophobic nature of a V substitution versus F. Moreover, nS-wt with Tis least effective among monovalents even at 50 μM. Eating of opRBCs is unaffected by the presence or not of 50 μM of the scrambled peptides nS-X and nS-XX. Consistent with all of these results with human macrophages, mouse-derived J774A. 1 macrophages show all of the same trends for nS -F,-V, wt, and -X peptides, including 3-fold more eating with F peptide relative to the minimal eating of opRBCs in the presence of X peptide (FIG. 6B).

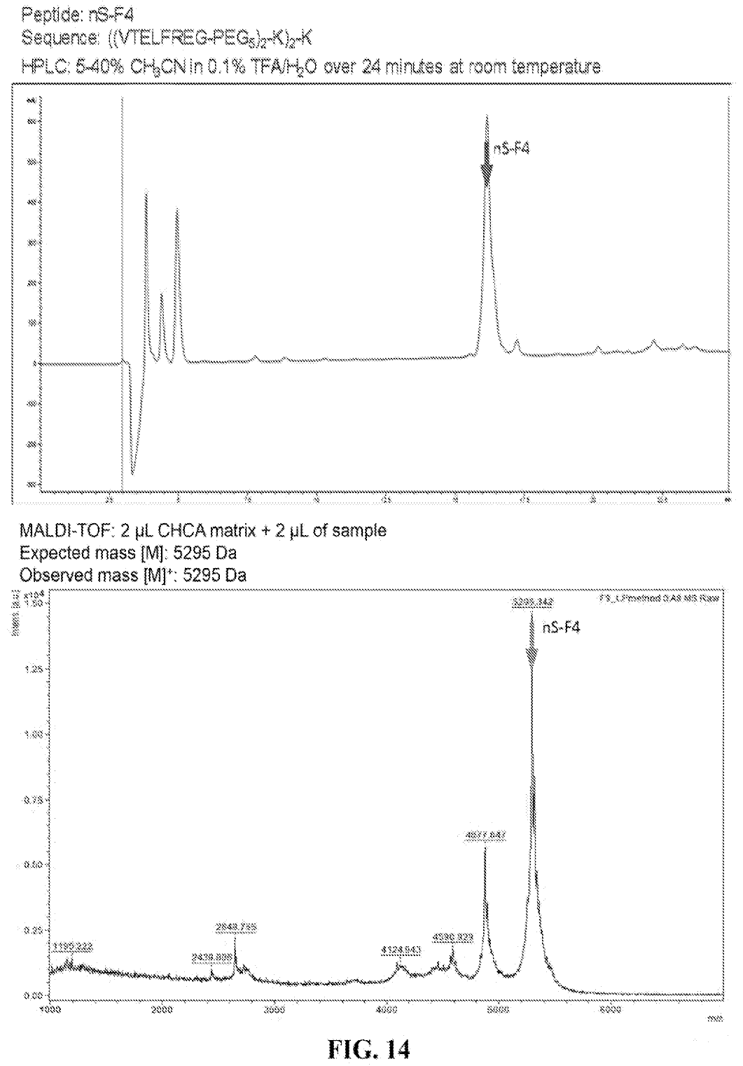

After observing and quantifying internalization of RBCs with the bivalent nS-FF peptide by macrophages, a tetravalent peptide was synthesized in order to study the effects of multivalency on cancer cells (FIG. 13 & FIG. 14). Interestingly, all nS peptides, except for scrambled nS-X and nS-XX, enhanced phagocytosis of RBCs opsonized by anti-RBC IgG (FIGS. 2D & 2F). nS-FF was effective even at 20 nM with ˜40% of peptide-treated macrophages showing at least one opsonized RBC internalized by the end of the 1 h assay, with an efficacy constant (Keff) of ˜ 8 nM indicating >100-fold higher activity than that of nS-wt (FIG. 2E). This key metric of efficacy in promoting phagocytosis follows the trend:

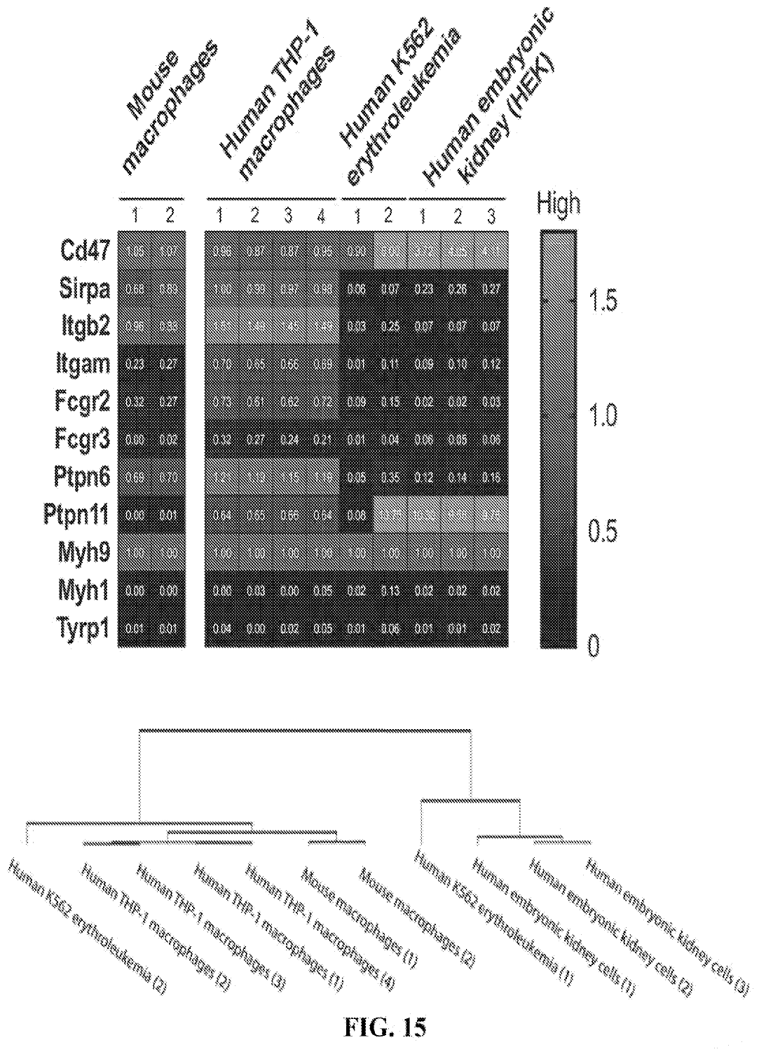

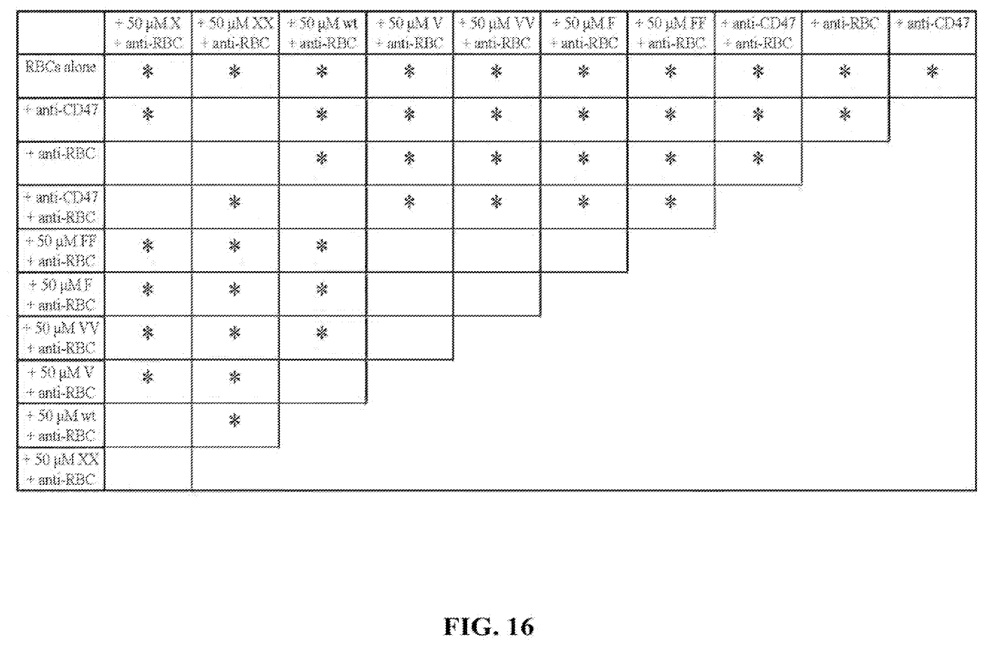

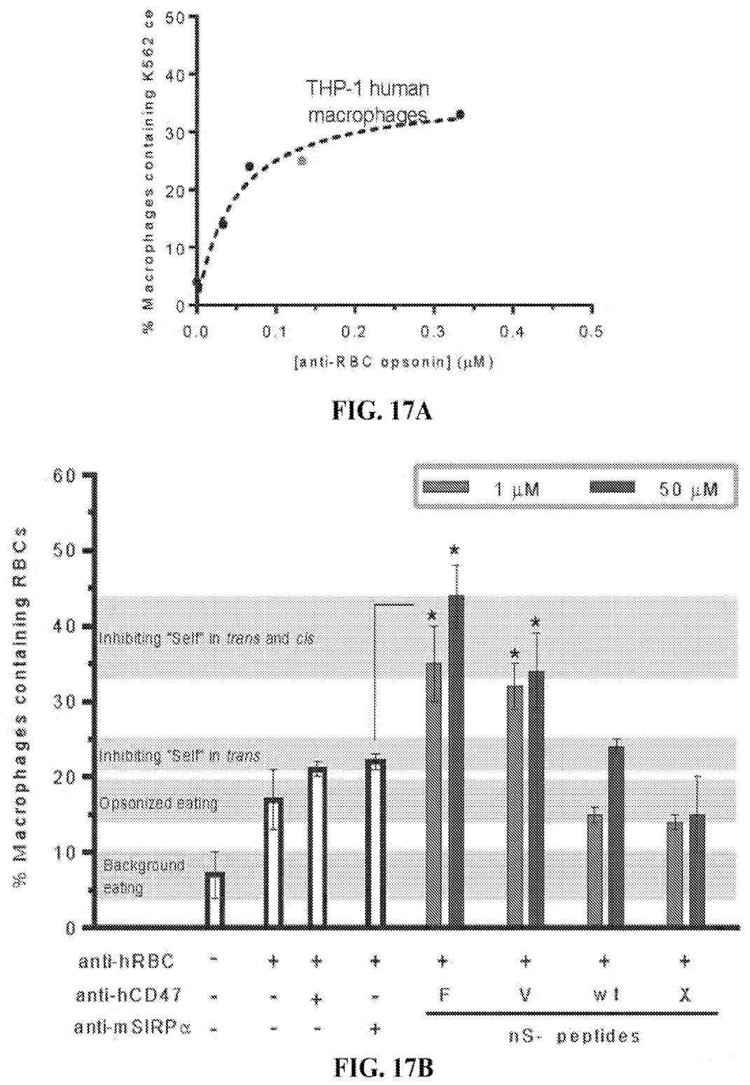

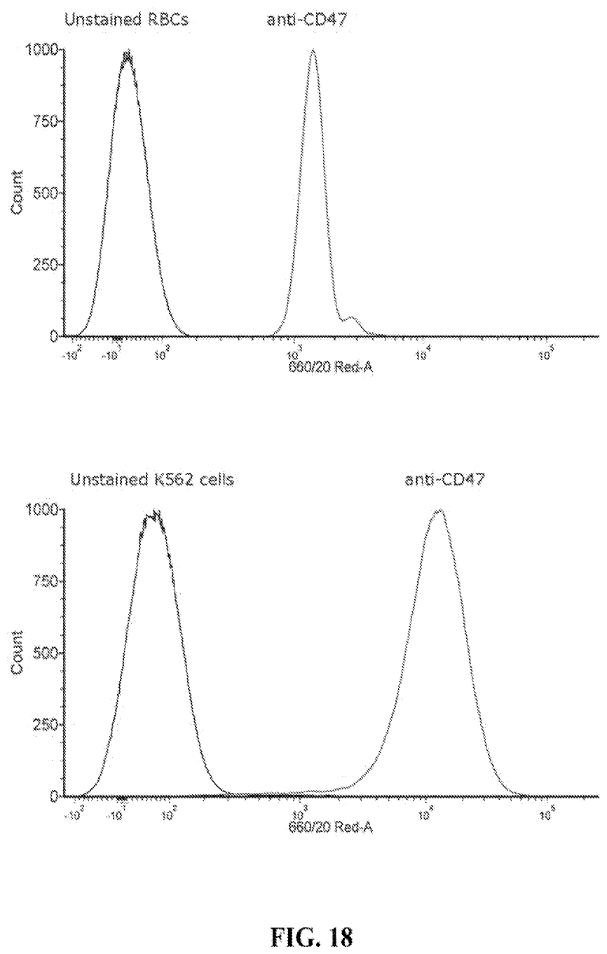

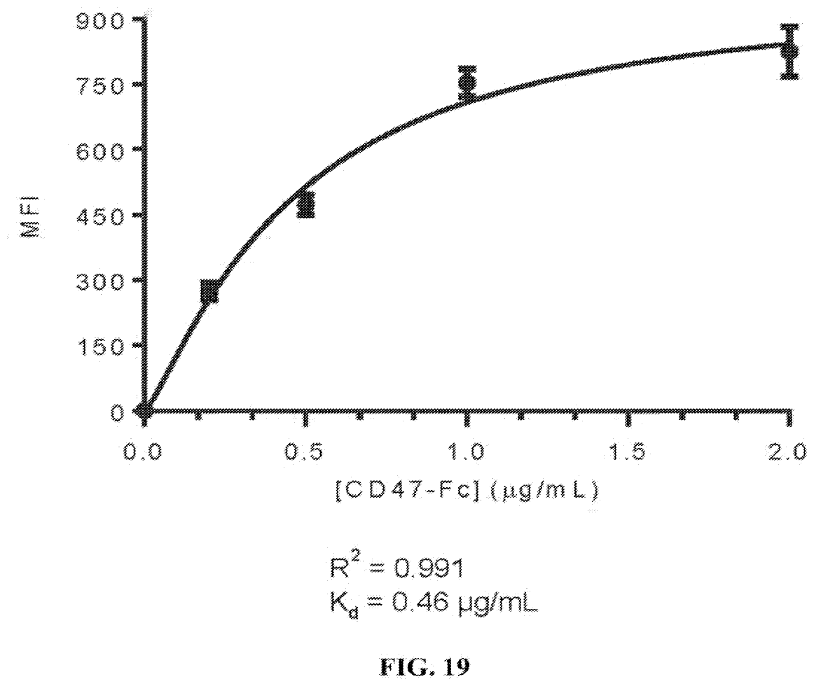

nS-FF >nS-F >nS-VV >nS-V >nS-wt (FIG. 15C)

Furthermore, maximum peptide concentrations of 50 μM show phagocytosis levels for nSwt and all nS-F and nS-V variants are well above those for nS-X and nS-XX controls that do not affect baseline engulfment of opsonized RBCs (FIGS. 2C & 2E). Maximum peptide concentrations reveal an additional effect when compared to anti-CD47 blockade. Combining anti-CD47 with anti-RBC causes ˜30% of macrophages to phagocytose opsonized RBCs, which is higher than the effect of anti-RBC alone (˜20% of macrophages contain opsonized RBCs; red open bar in FIG. 2D), consistent with anti-CD47 inhibiting recognition by the macrophage's SIRPα. Likewise, combining nS-wt with anti-RBC also causes ˜30% of macrophages to internalize opsonized RBCs. Note that saturating amounts of anti-CD47 on RBCs has minimal effect on baseline engulfment (˜5-10% of macrophages) and that anti-RBC is always used at ˜133 nM (FIG. 6A) Surprisingly, the highest levels of phagocytosis - with ˜40-50% of macrophages containing opsonized RBCs - are seen for all F and V substituted peptides at maximum peptide concentrations (FIG. 2D).

Opsonized erythroleukemia K562 cancer cells were similarly tested for phagocytosis in the presence of the most potent nS-FF and the tetravalent nS-F4 peptides. The anti-RBC successfully opsonized and triggered phagocytosis of K562 cells by macrophages (FIGS. 2A, 2F and 2E). Multivalent nS-FF and nS-F4 nearly doubled the percentage of phagocytic macrophages relative to trans anti-CD47 blockade whereas macrophages were unaffected by nS-XX control (FIG. 2F). Interaction of the nS peptides with K562 cells is likely minimal due to the relative lack of SIRPα expression. However, saturation at ˜40% of macrophages engulfing opsonized K562 cells is less than the ˜50% for opsonized RBCs, perhaps because K562 cells have more CD47 molecules than RBCs, suggesting more “don't eat me” signaling. For both opsonized RBCs and opsonized K562s (FIGS. 2D, 2F), the hyperphagocytosis that is achieved with soluble F- and V-nS peptides matches the recently measured increases for disruption of both (i) the trans interactions between CD47 on a target and macrophage SIRPα and also, importantly, (ii) the cis interactions between CD47 and SIRPα on the same macrophage (FIG. 2G-left). Expression profiling of macrophages confirms these cells generally express CD47 at roughly similar levels to other cell types (FIG. 15). A recent study showed that macrophage-CD47 knockdown removed a basal level of inhibitory ‘self’ signaling from the cis interaction and thereby caused hyper-phagocytosis, with similarly increased phagocytosis when anti-CD47 was added to macrophages separate from adding anti-CD47 to block the target. A preliminary conclusion is that nS-FF and the other substituted peptides, which were designed to bind SIRPα, inhibit both the cis and trans interactions between SIRPα and CD47 (FIG. 15G-right).

Consistent with the results with human macrophages, mouse-derived J774A.1 macrophages show all of the same trends for nS-F, nS-V, nS-wt, and nS-X peptides, including 3-fold more phagocytosis with nS-F relative to the minimal internalization of opsonized RBCs in the presence of nS-X control (FIG. 6B). The ˜10-20% increase in phagocytic mouse macrophages with F and V substituted nS peptides relative to anti-CD47 on opsonized

RBCs agrees with the cis and trans inhibition effect, and the nS-wt peptide matches anti-CD47 blockade effects—at least for high concentration (50 μM). Indeed, the effective activity of nS-wt is ˜100-fold weaker in the mouse macrophage assay than in the human assay. The difference could reflect a singular difference between the 8-residue sequences of human and mouse CD47′s: the Thr in human-CD47 is replaced by a less bulky and less hydrophobic Ser in mouse-CD47, which again affirms that sequence matters.

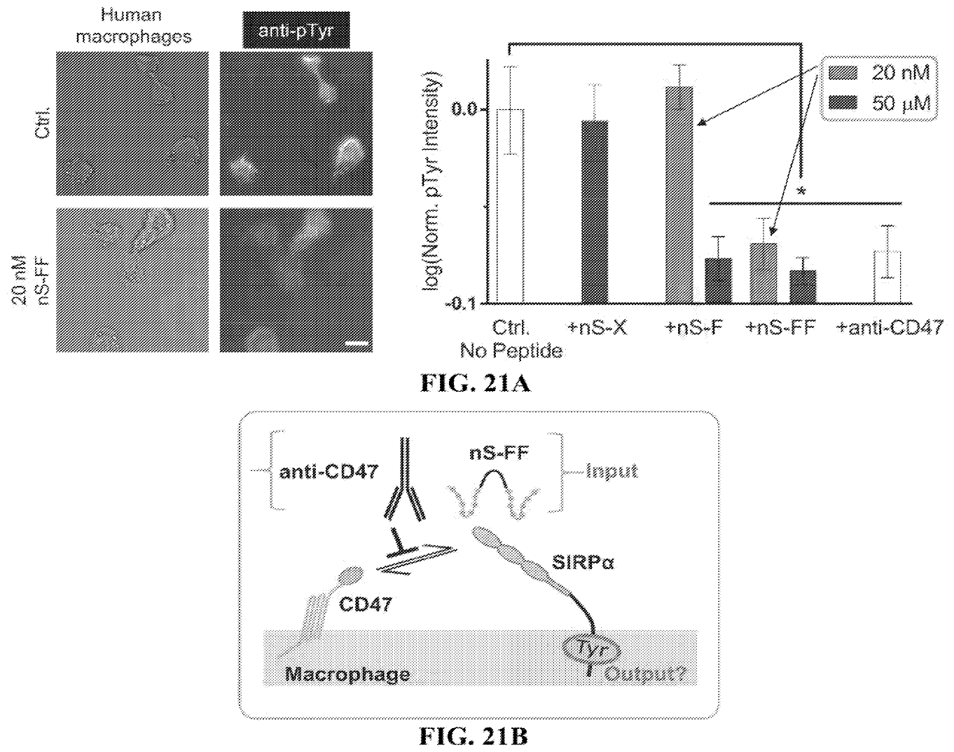

The various phagocytosis assay results lead to the determination of certain findings (FIG. 2C and FIG. 21). In certain embodiments, nS-F peptides bind competitively to the macrophage surface. In other embodiments, nS-F peptides affect phosphotyrosine levels that are known to be modulated by the binding of CD47 to SIRPα. In certain embodiments, nS-F and -V peptides have structures more suited to SIRPα's binding pocket than the -X peptide.

### Example 3: Multivalent nS Peptides Inhibit CD47-Fc Binding to Human Macrophages

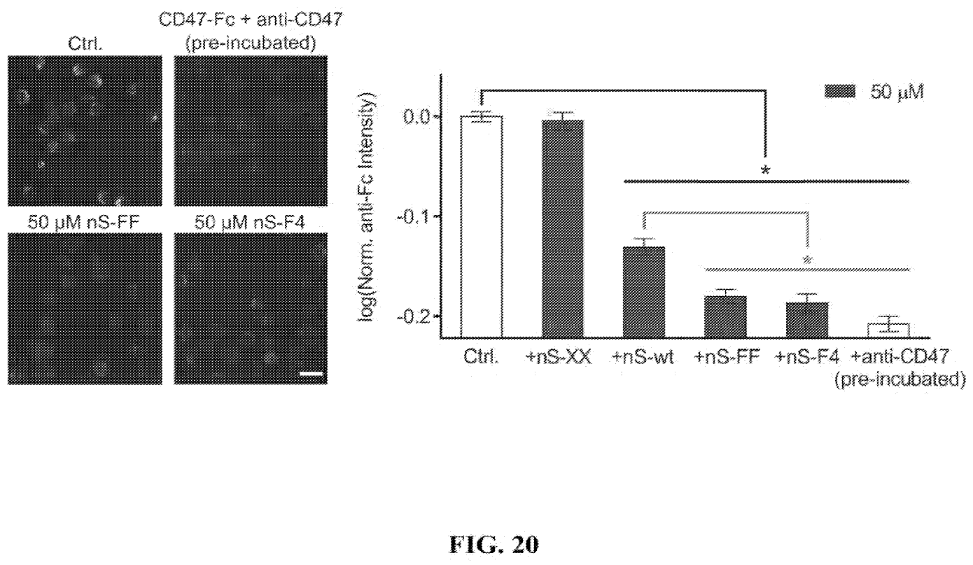

To determine whether the nS peptides bind to SIRPα, nS-FF and nS-F4 were used as soluble competitive inhibitors of saturable CD47-Fc fusion protein binding to macrophages (FIG. 20). Addition of multivalent nS peptides was followed by Fc-receptor blockade to minimize Fc-driven binding of construct. Afterwards, CD47-Fc was added and then finally anti-Fc fluorescence imaging was performed (FIG. 20, left). Quantitation of fluorescence shows the expected trend for the levels of inhibition:

nS-F4˜nS-FF >nS-wt >nS-XX (=0)

Anti-CD47 was pre-incubated with CD47 as a positive control for inhibition of binding to SIRPα. 151 This showed that both nS-F4 and nS-FF are as inhibitory as anti-CD47.

### Example 4: Tyrosine Phosphorylation in Macrophages is Suppressed by nS Peptides

Given that the interaction of CD47 with SIRPα initiates a de-phosphorylation cascade regardless of whether the interaction occurs in trans or in cis, two key nS peptides were again added to the macrophages for quantitative fluorescence microscopy. Basal levels of phosphotyrosine (pTyr) in wildtype macrophages are indeed suppressed by nS-FF and by anti-CD47 (FIG. 21A). Importantly, nS-FF suppressed pTyr signal at the low peptide concentration (20 nM; FIG. 21A) that maximizes phagocytosis by blocking both trans and cis interactions (FIG. 2C). Monovalent nS-F at the high concentration (50 μM)—that likewise maximized phagocytosis—also suppressed pTyr, whereas 20 nM nS-F did not significantly affect pTyr, consistent with blocking only trans interactions at the low concentration (FIGS. 2C & 2D). The negative control peptide nSX (50 μM) had no effect on pTyr, which is consistent with the lack of effect in phagocytosis. Note that anti-CD47 in the phagocytosis studies was added to the opsonized RBCs and K562 cells blocking only the trans interactions, whereas in these pTyr experiments (FIG. 21A), anti-CD47 was added to the macrophages to determine the effect on basal signaling in the absence of phagocytosis. The pTyr results are not only consistent with peptide disruption of cis interactions between CD47 on the surface of the macrophage and SIRPα (FIG. 21B), but further underscore the functional potency of multivalent nS peptides.

### Example 5: Competitive Binding of nS Peptides to Macrophages

In order to first determine whether the nS peptides interact with SIRPα, polystyrene streptavidin beads were incubated with biotinylated nS peptides and then with recombinant SIRPα. Streptavidin was stained with anti-streptavidin-FITC and SIRPα was stained with anti-SIRPα-APC. Indeed, SIRPα were observed to be on beads only with prior incubation of nS peptides (FIGS. 3 & 7). One then tested for increased affinity of the bivalent variants to SIRPα relative to their monomer counterparts by setting up an inhibition assay with unlabeled bivalent peptides (nS-FF/nS-VV) and FAM-labeled nS-F (FIG. 3A). nS-F-FAM intensity on the surface of THP-1 macrophages is significantly less than nS-X-FAM and is further decreased when THP-1 macrophages are pre-treated with either nS-FF or nS-VV (FIG. 3B).

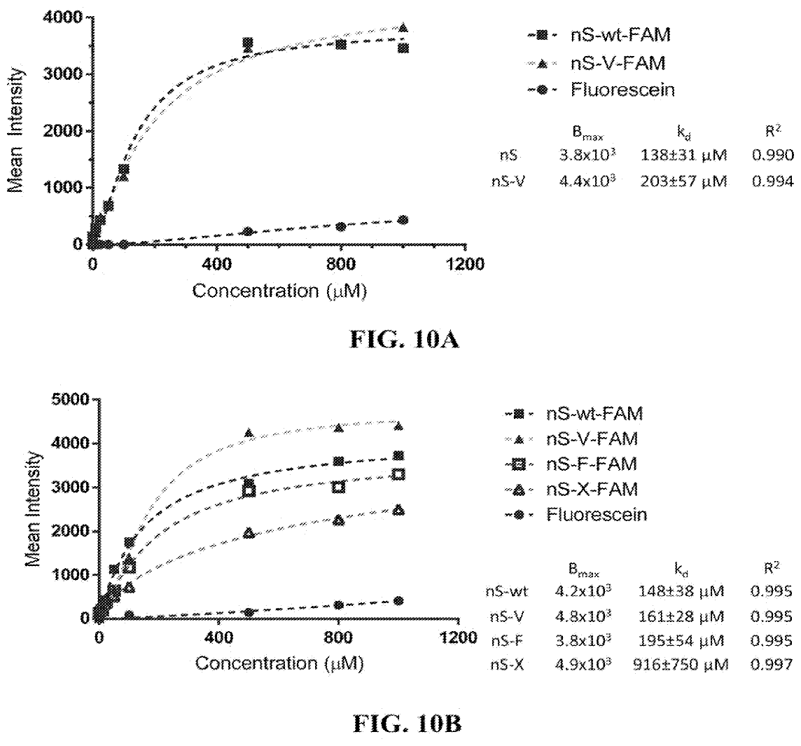

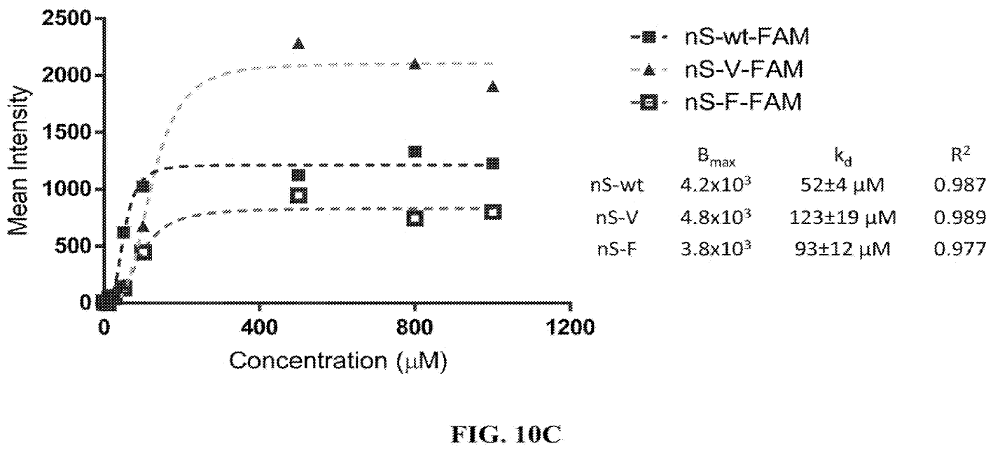

To assess the extent of peptide interaction with SIRPα, the binding of the peptides was compared to that of anti-mouse SIRPα-FITC (P84-FITC) on J774A.1 macrophages. While binding was observed on the surface of the macrophages, binding of nS-wt and nS-V was not uniform around the membrane of all the cells. For most of the cells, nS-wt and nS-V bound to the edges of the macrophages. nS-X stained the entire cell surface of all macrophages (FIG. 8). Similar observations and results were observed when compared to THP-1 macrophages. The same binding experiment was repeated on live J774A.1 and THP-1 macrophages (FIG. 8). The peptides appeared to predominantly bind to one side of the macrophage. This form of binding was observed for all fluorescently labeled peptides except for nS-X which stained the entire cell. For nS-X, however, fluorescence intensity was significantly lower than the other peptides. Apparent binding affinities were determined to be on the order of 30 μM with fixed cells (FIGS. 9) and 100 μM with live (FIG. 10).

In order to visualize the uneven binding of the peptides to the surface of the macrophages, a live staining experiment of THP-1, J774A.1, and A549 cells, which all express SIRPα at different levels, was performed with FAM-labeled nS-wt and nS-X and analyzed by confocal microscopy (FIG. 11). At 100 uM, confocal imaging revealed that nS binds the surface of THP-1 and J774A. 1 macrophages and is also internalized. nS-X does not bind the surface of the macrophages as efficiently but is also internalized at the same levels as nS-wt. At lower concentrations (1 μM) confocal images show that the majority of nS-wt binds to the surface of the macrophages. Fluorescence of nS-X was consistently lower on THP-1 and J774A.1 macrophages relative to nS-wt. To determine whether this binding is due to the presence of SIRPα and that nS-wt binding was not non-specific, the binding of the peptides was tested on A549 (SIRPα positive) and COS-1 (SIRPα negative) cells. nS-wt binding was observed at 100 uM on A549 cells all around the surface. Peptide fluorescence was not observed inside the cell indicating that the peptides do not penetrate the cell membrane. The level of SIRPα expression is lower on the surface of A549 cells when compared to THP-1 and J744A.1 macrophages, which was confirmed with the lower fluorescence intensity of nS-wt. Very low signal resulted from decreasing the concentration of nS-wt to 1 μM. nS-X fluorescence was not observed at either concentration on A549. Binding of nS-wt to COS-1 was primarily non-specific as the fluorescence was sparse and only on the surface of the cells. All binding was compared to fluorescein as a control on all cells which stained cells minimally.

### Example 6: Phosphorylation Levels in Macrophages are Suppressed by nS Peptides

In order to further relate these peptides to inhibiting the CD47—SIRPα axis, phosphorylation levels were observed on THP-1 macrophages with and without peptides. The engagement of CD47 with SIRPα results with phosphorylation of SIRPα's cytoplasmic tail inhibiting phagocytosis whether this interaction occurs in trans or in cis. This was confirmed as basal levels of phosphorylation were observed in wild type THP-1 macrophages (Ctrl in FIG. 21A) which was suppressed after the addition of anti-CD47 ([+anti-CD47] in FIG. 21A). It was tested whether nS-F and nS-FF inhibit phosphorylation. Indeed, once the peptides are bound to the macrophages, phosphorylation levels significantly decrease. Phosphorylation levels were restored when macrophages were treated with 20 nM of nS-F. On the contrary, 20 nM treatment of nS-FF efficiently suppresses phosphorylation indicating multivalency plays a major role in interacting with macrophages ([+ns-FF] in FIG. 21A). These results are consistent with the phagocytic effects observed for the respective peptides (FIG. 2) suggesting that these peptides bind to SIRPα and block cis interactions with CD47 present on the surface of the macrophages (FIG. 21B), as well as trans interactions between CD47 on RBCs and adjacent THP-1 macrophages.

### Example 7: nS Peptides are Mainly Disordered but eXhibit Some Hairpin Structure

The potential for the 8-amino acid peptides to fold into a β-hairpin in solution was investigated. CD measurements (FIGS. 4A-4B and 12A-12B) were conducted on all the peptide sequences in order to elucidate the secondary structure of the peptides. At 5° C., the peptides seem to exhibit a random coil conformation with some B-turn structure. Namely, positive ellipticity is observed around 215-220 nm for all peptides—except nS-X and nS-XX—and negative ellipticity around 195 nm. The scrambled peptides are primarily random coils in solution. Melting the peptides at 90° C. results in loss of all positive ellipticity indicating loss of structure. Interestingly, the bivalent peptides also have some secondary β-hairpin structure (FIG. 4A). Difference plots show that the multivalent nS-FF and nS-VV structures agree, whereas nS-XX has less of a hairpin structure consistent with structure rationale from the phagocytosis results.

Because of the snug fit of a key CD47 β-hairpin within SIRPα, the secondary structures of the 8-amino acid peptides in solution was investigated using circular dichroism (CD) (FIGS. 12 & 4). At low temperature (5° C.), the peptides are largely random coils with a minor fraction of B-turn structure when compared to other short peptides. This is evident for the most functional peptide nS-FF as a slight positive ellipticity peak at 215-220 nm and deep negative ellipticity peak around 195 nm—signals that are somewhat clear for the slightly less functional nS-VV peptide but much attenuated for nS-XX or nS-X. Thermal unfolding at 90° C. is evident in suppression of the ellipticity peaks. Difference spectra (FIG. 4B) are the same for the bivalent nS-FF and nS-VV, whereas nS-XX is attenuated—showing a trend similar to the phagocytosis results (FIG. 2). The nS peptides here are mainly random coils (FIG. 12), which suggests an induced fit association with SIRPα on macrophages. Phagocytosis levels of nS peptide treated macrophages were compared to anti-CD47 blockade of the target, with bivalent nS-FF and tetravalent nS-F4 proving to be more potent in enhancing phagocytosis at pharmacologically relevant concentrations (20 nM) (FIG. 20).

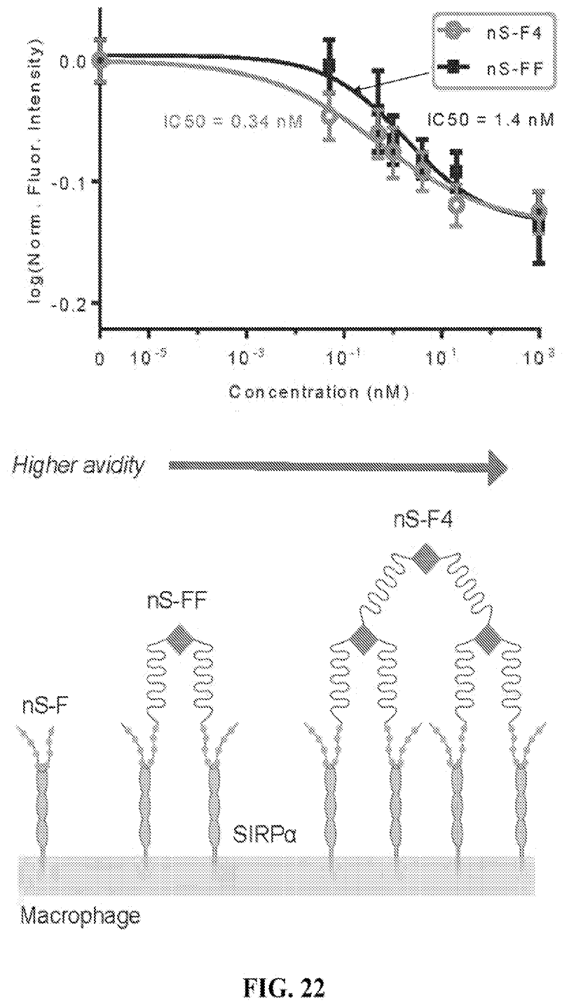

The slight increase of phagocytic macrophages when cultured with nS-F4 versus nS-FF (FIG. 20) seems consistent with increased avidity as a soluble inhibitor (FIG. 22), which supports an advantage of multivalency, even though the effects plateau. The increased levels of target phagocytosis are consistent with cis and trans inhibition, and nS-FF's suppression of phosphorylation in isolated macrophages is consistent with blocking of cis binding of SIRPα to CD47 (FIGS. 21A & 21B).

### Example 8: Safety of ns-FF Injections in a Pre-Clinical Trial

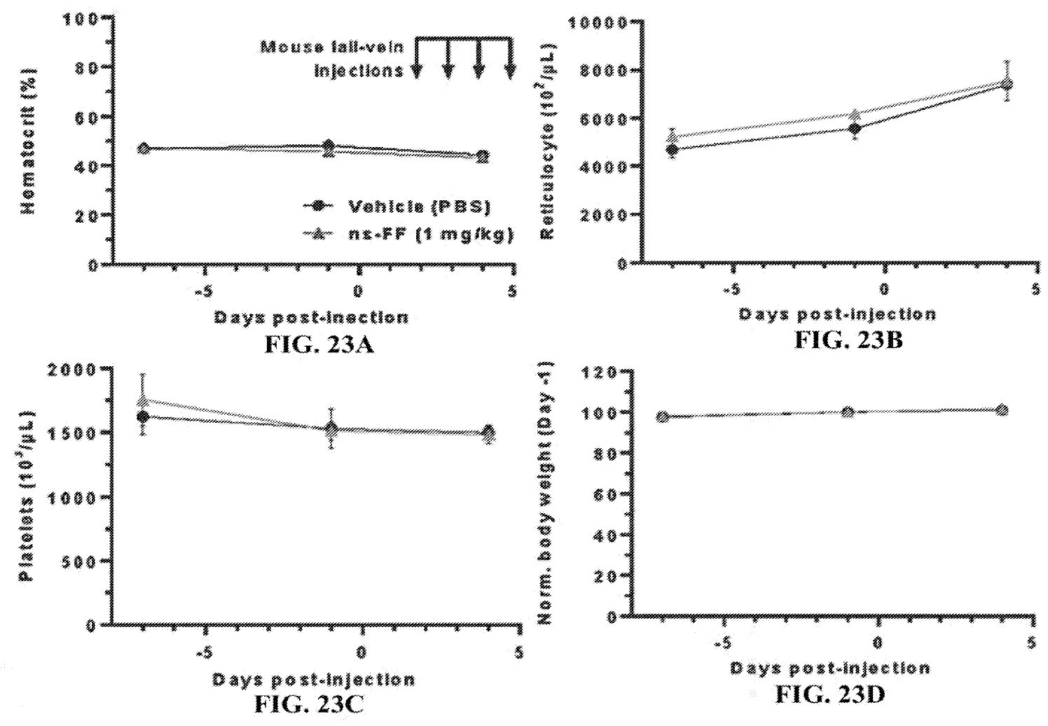

Phase 1 clinical trials for safety of anti-CD47 in patients have shown that infusion into the bloodstream decreases RBC numbers (i.e. hematocrit) and increases reticulocytes (i.e. new RBCs), and related blood safety concerns apply to a bivalent CD47-binding protein made with SIRPα domains fused to a macrophage-binding domain (Fc domain). Given that the nS-F peptide of the present disclosure increases phagocytosis of opsonized RBCs and also associates with mouse macrophages, the safety of the more potent nSFF peptide by intravenous injection into mice was assessed. Overall, nS-FF in PBS showed no differences versus PBS vehicle control in its effects on mouse hematology and body weight after four daily tail-vein injections of 1 mg/kg peptide (FIG. 23). This corresponds to about 8 μM in the blood, assuming rapid mixing and no dilution within the ˜1.5 mL blood volume of the mouse. Withdrawal of ˜140 μL from this blood volume was necessary to obtain a complete hematology profile, and such a volume is expected to cause slight decreases in hematocrit and platelets as shown (FIGS. 23A-23B). Consistent with this loss, more RBCs should be produced in each mouse to compensate for the RBC loss, and the ˜30% increase in reticulocytes after the first two blood draws over 11 days (FIG. 23C) is similar to the prior amount of blood removed (˜300 μl/1500 μl). The mice (˜14 weeks) also continued to gain weight at the same rates (˜5% over 11 days) regardless of peptide injections. These changes are thus expected but, importantly, unaffected by the nSFF peptide, which establishes some safety.

### Example 9: Selected Discussion

Immunotherapies such as the development of genetically modified chimeric antigen receptor T-cells (CAR-T cells) utilize immune cells from the same patient enhancing the clearance of tumor cells. While results with such therapy has been dramatic against cancer and received FDA approval for use as a treatment, serious side effects remain. Adapting SIRPα-blocked macrophages harvested from patients poses a potentially safer alternative where targeting antibodies or peptides are cleared after time leaving the unmodified macrophages in circulation. Macrophage immunotherapy also has an advantage over T-cells in clearing solid tumors. Furthermore, the nano—Self peptides of the present disclosure add more advantages over antibodies as they are much smaller but are similar in terms of potency. Rationally designing CD47-inspired peptides introduces a new direction in developing macrophage checkpoint inhibitors.

Peptide-based therapies are numerous, and for cancer, they include approved analogs of naturally occurring molecules (e.g. bortezomib, carfilzomib, and goserelin). In cell adhesion signaling for example, the tripeptide RGD derived from extracellular matrix led to a synthetic analog with increased affinity for matrix receptors and with utility as a soluble competitive inhibitor of adhesion in clinical trials against cancer. Peptides are usually synthesized at low cost (˜$1/mg here) and can be stored at high concentration relative to therapeutic IgG's. To be clear, lab-grade anti-CD47, anti-SIRPα, and anti-PD1 are ˜$100/mg, and clinical grade antibodies such as anti-PD1 cost >$100 K/patient/year. Moreover, very few residues in a ˜150 kDa antibody physically contact a target antigen.

The 21-amino acid ‘Self’ peptide was the first peptide shown to bind SIRPα and recapitulate the anti-phagocytic signaling of full length CD47. Although a similar 21-amino acid peptide was reported to not bind soluble SIRPα,55 the 8-amino acid nano—Self peptides disclosed herein (1) enhanced phagocytosis of antibody-opsonized human cells (normal and cancer) by human macrophages and (2) were as effective as anti-CD47 in inhibiting a CD47-Fc construct in binding to human macrophages. Without wishing to be limited by any theory, a non-limiting concept of the nS peptides is to bind and inhibit SIRPα rather than target CD47. Anti-CD47 infusions in the clinic show some efficacy against opsonized liquid tumors but also cause anemia, which is not evident in initial studies here and could in part reflect the Fc function of anti-CD47.

When displayed on particles, the 21-amino acid ‘Self’ peptide inhibited phagocytosis of opsonized particles, whereas the soluble peptides here function as antagonists consistent with prior use of large, soluble CD47 ectodomain as inhibitors of SIRPα to enhance phagocytosis of tumor cells. In certain embodiments, the smaller peptides here are more likely to penetrate a tumor from the circulation, or they might be delivered to tumors (which are typically rich in macrophages) by various methods that range from nanoparticle-mediated ‘nano-gene’ therapy to packaging them into either peptide-secreting bacteria or backpacks that attach to tumor-injected macrophages. Furthermore, anti-CD47 can directly opsonize cells and cause engulfment by macrophages, because the antibody's Fc domain activates the Fc receptor (FcR) on the macrophages—although studies have shown that the B6H12 clone of anti-CD47 does not greatly stimulate phagocytosis. Other studies have demonstrated that a bivalent anti-CD47 nanobody that lacks an Fc-domain caused modest anemia and mild thrombocytopenia in mice (following a similar injection and bleeding protocol as used here) but addition of an Fc domain increased the adverse effects. Importantly, the nS peptides of the present disclosure lack an activating Fc domain and should solely antagonize SIRPα, eliminating opsonization, and thereby minimizing clearance of healthy cells.

In sum, and without wishing to be bound by theory, synthesis and functional tests of multivalent, CD47-inspired nano—Self peptides with hydrophobic substitutions at a central Thr demonstrate potential as a nanomolar agonist for phagocytosis of targeted diseased cells such as cancer cells. Sequence analyses of various species beyond human and mouse indicate the nS peptides can function with macrophages in monkey and dog, which are important species for evaluation of safety and efficacy.

### Example 9: cyclic nS Peptides Efficiently Enhance Macrophage-Mediated Phagocytosis of Target Cells

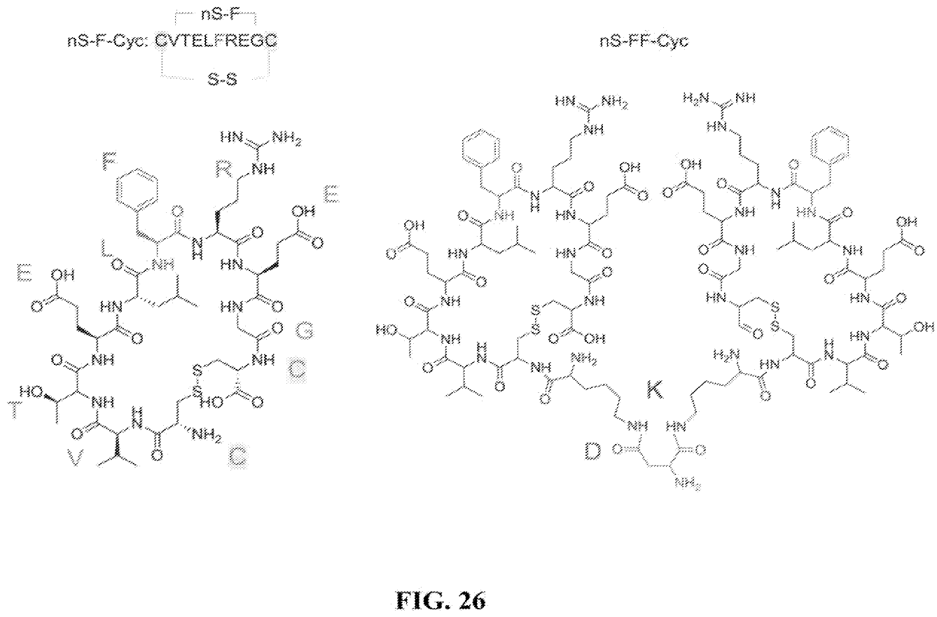

Peptide inhibitors against the CD47—SIRPα axis exist and continue to emerge. In the preceding examples, multivalent linear peptides were demonstrated that inhibit SIRPα which motivates the development of more peptide agonists, in particular cyclic peptides. These types of peptides have advantages such as higher affinity and selectivity for protein receptors when compared to linear peptides due to their limited conformational flexibility and higher surface area. Cyclic peptides are also more resistant to enzyme degradation due to the lack of free termini and more stable cores, a result of increased intramolecular interactions. As such cyclic nano-Self peptide (nS-Cyc) were designed based on the wild type nano-Self (nS-wt) peptide of the current invention by bridging the ends with a disulfide bond and verified it's synthesis by MALDI-TOF mass spectrometry (FIG. 26A). While some linear nS peptide sequences are found in proteins expressed mostly in bacteria, no organism was found to express a ribosomal protein that contains the nS-Cyc sequence (FIG. 26A Inset Table). A kinase protein expressed in rosary peas contains the closest sequence to nS-Cyc (FIG. 26B), and while this plant is toxic, toxicity seems unrelated to the aligned protein suggesting low immunogenicity (FIG. 26B).

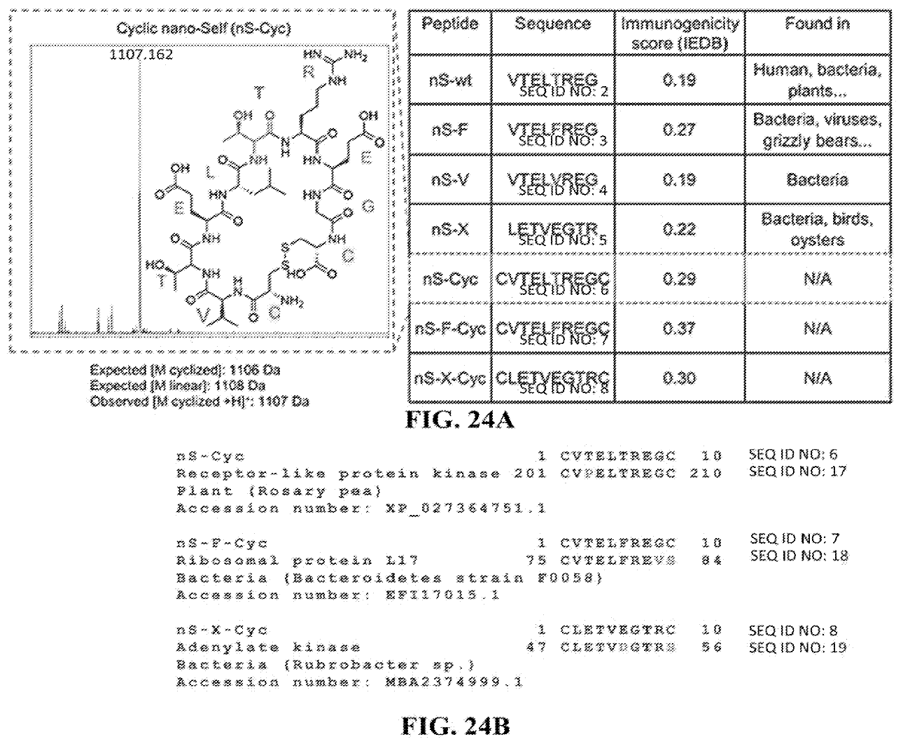

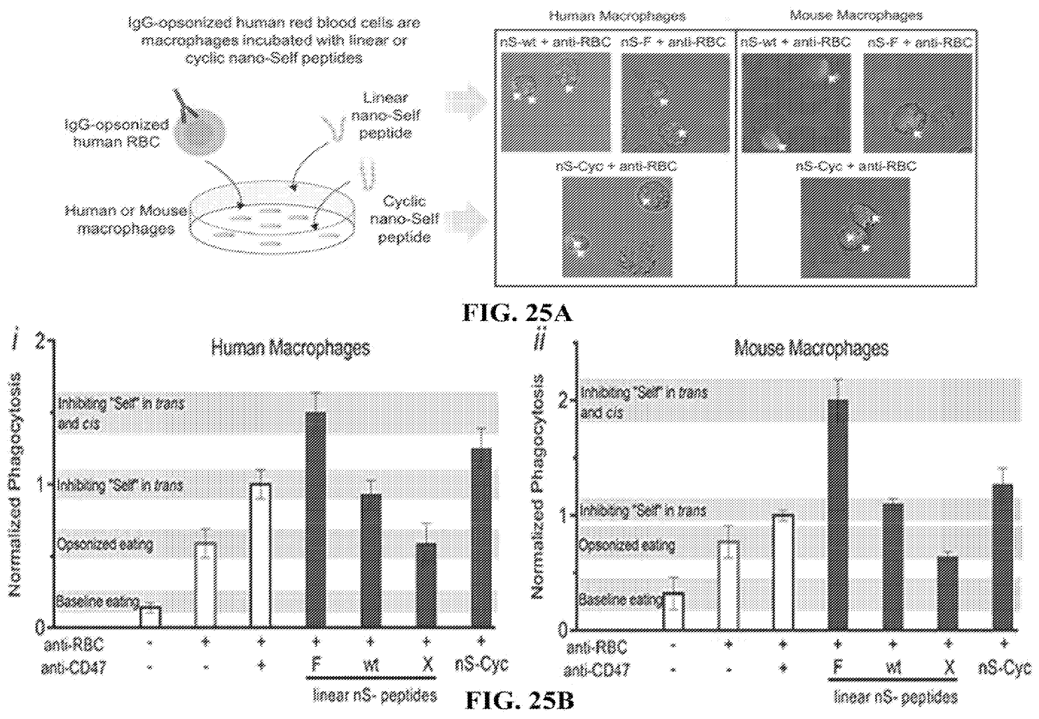

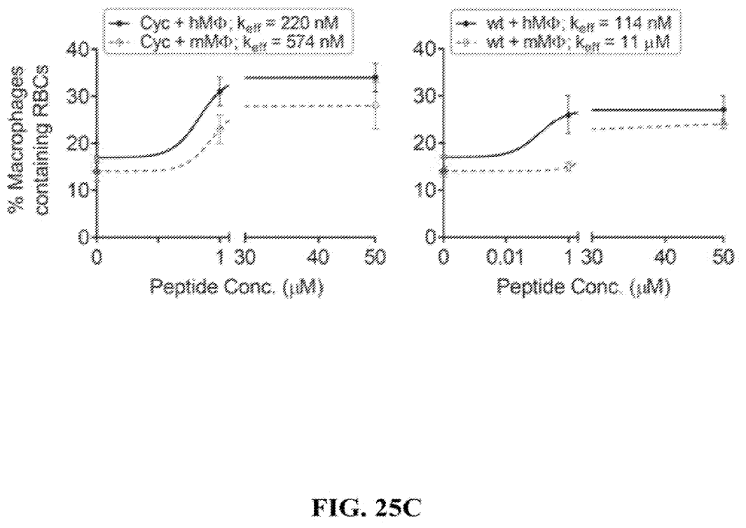

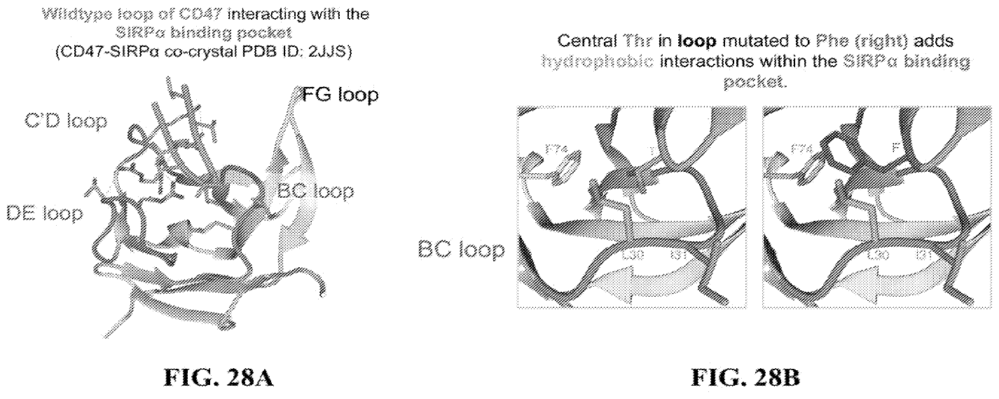

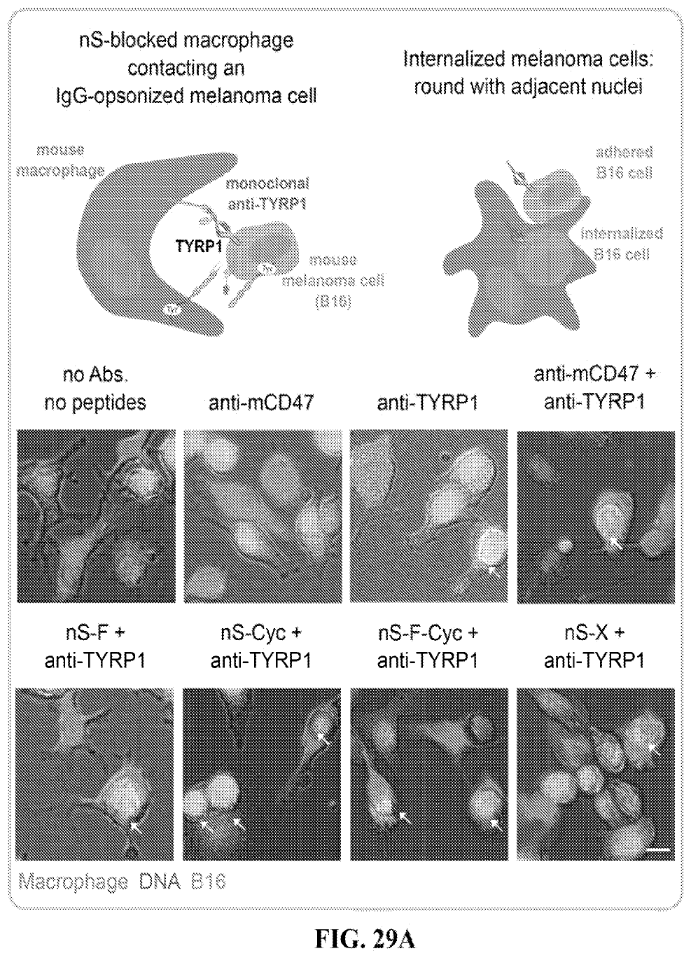

The co-crystal structure of CD47—SIRPα shows the highest density of CD47 contacts localizes to a β-hairpin loop that docks into a hydrophobic pocket of SIRPα (FIG. 28A). Despite additional interactions outside this binding site, it was hypothesized that cyclic peptides could be made based on this eight amino acid loop and that the synthetic nS-Cyc peptide would maintain activity. Furthermore, a Thr residue in the center of the β-hairpin shows multiple contacts with SIRPα's hydrophobic pocket (FIG. 28B) leading us to hypothesize that mutating the Thr to a more hydrophobic Phe residue could strengthen the interactions of nS peptides with SIRPα (FIG. 24A). Terminal Cys residues allowed for cyclization via disulfide formation, and a scrambled nS-X peptide was also made as a control. The particular design of the nS-Cyc excludes two Glu residues that normally flank the eight residue wildtype CD47 sequence (FIG. 32), and it was postulated that repulsion of these two negative charges undermines binding to SIRPα as observed with a similar cyclic peptide that included these two Glu residues.

Cyclic and linear nS peptides were synthesized on rink amide resins resulting in C-terminal amide groups. This modification also eliminates a negative charge. Matrix assisted laser desorption ionization-time of flight (MALDI-TOF) results proved consistent with the predicted mass-to-charge ratios (m/z), and analytical HPLC confirmed >98% peptide purity (FIGS. 24A, 33).

### Example 10: nS Peptides Increase Macrophage Phagocytosis of Antibody-Opsonized Cells

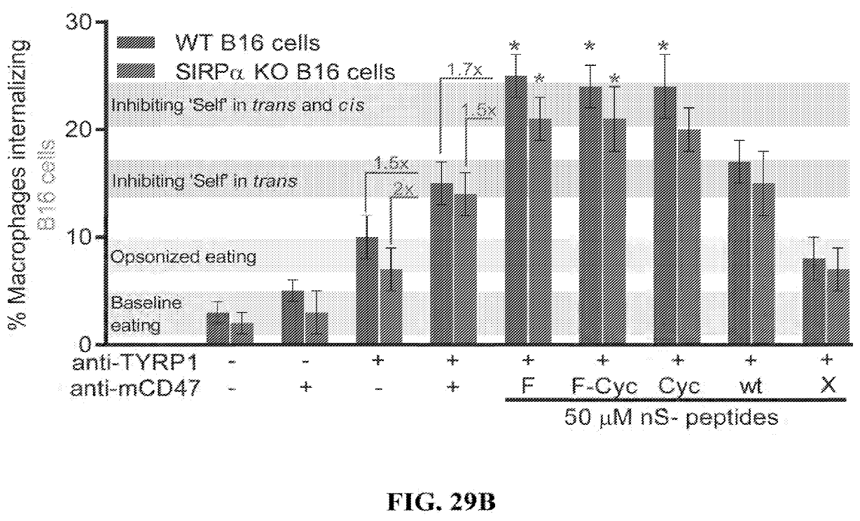

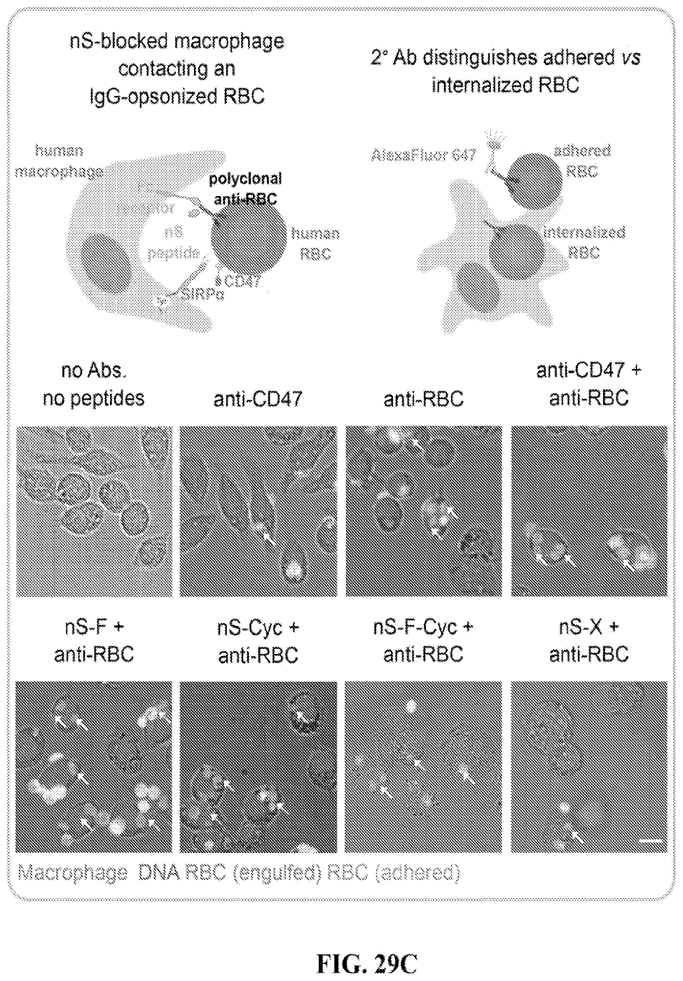

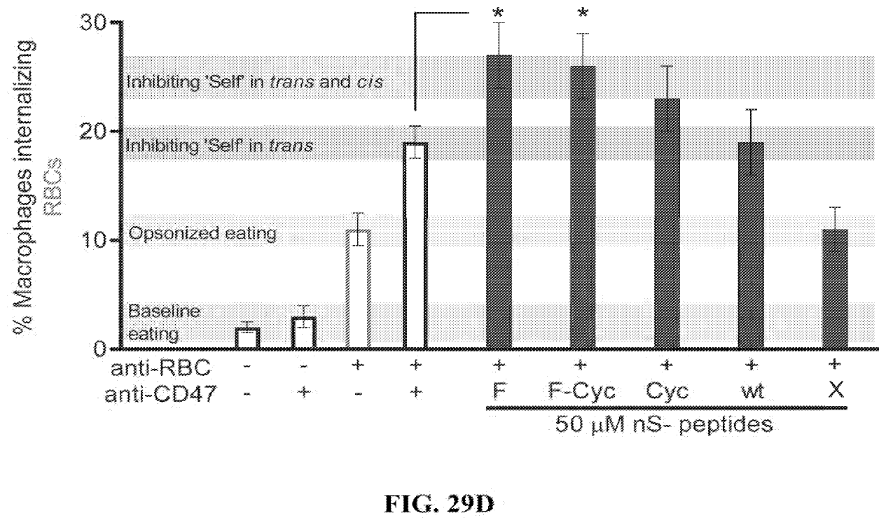

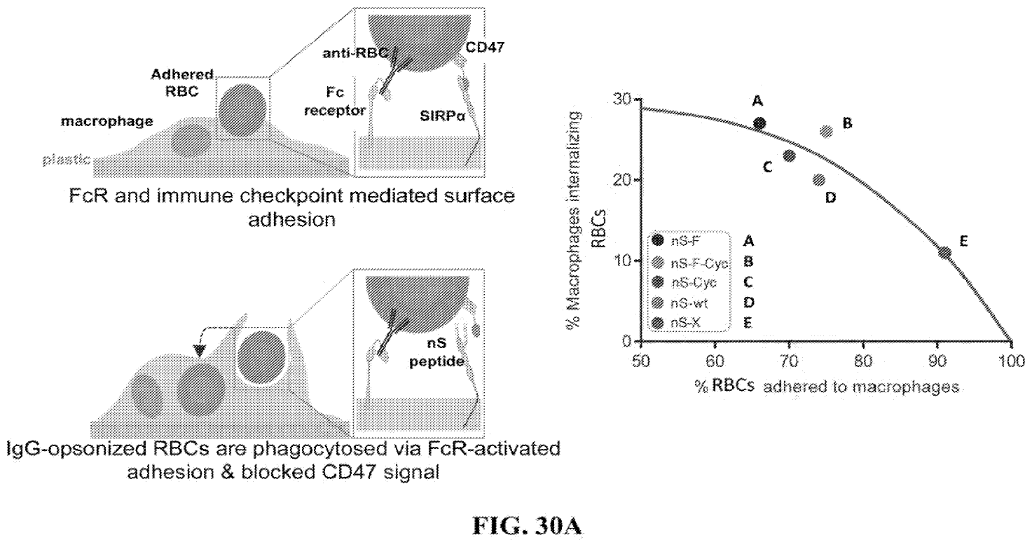

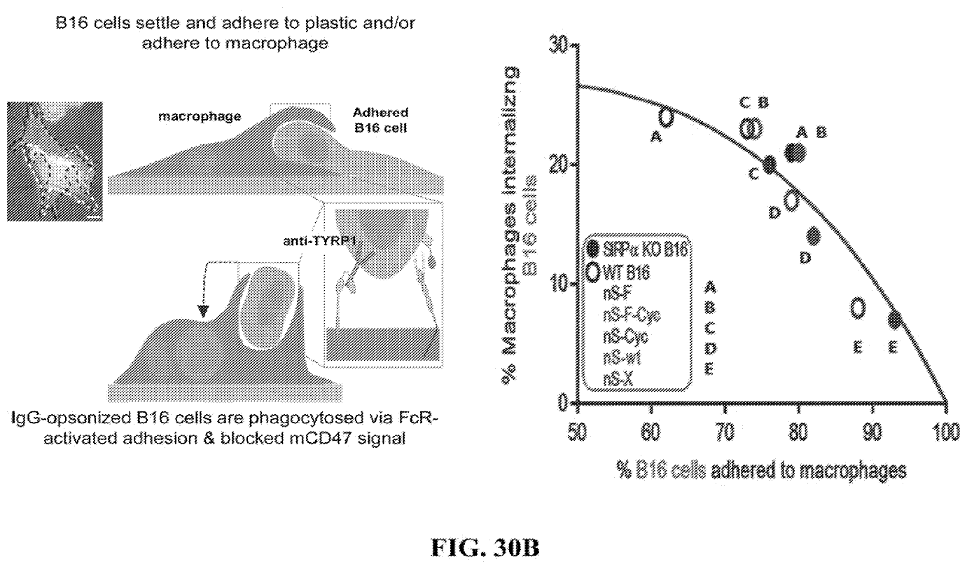

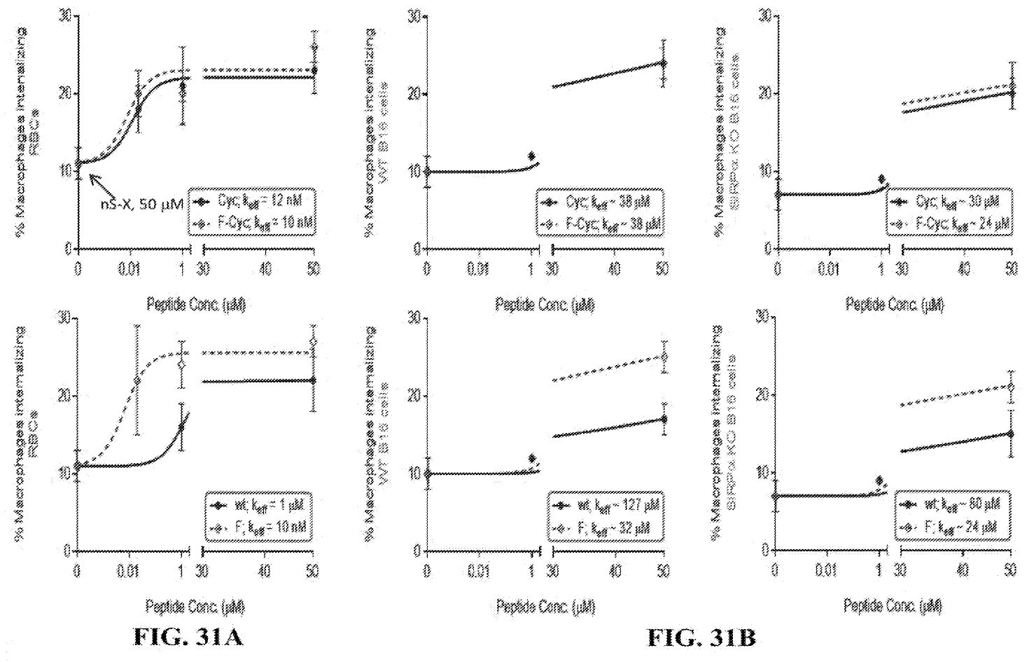

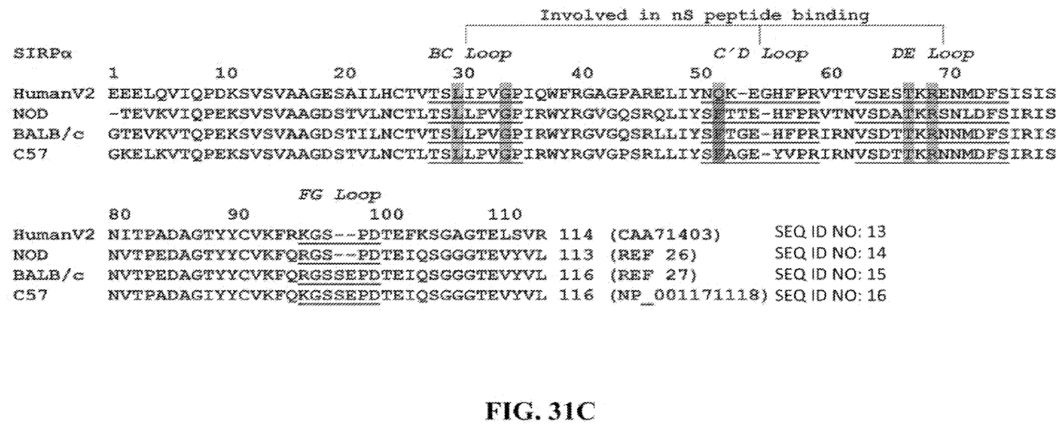

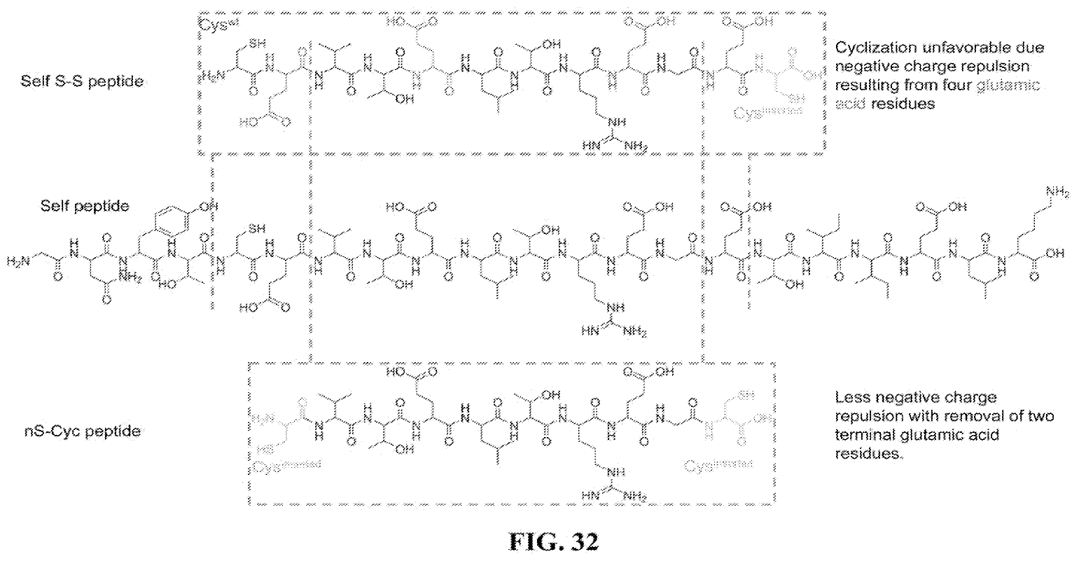

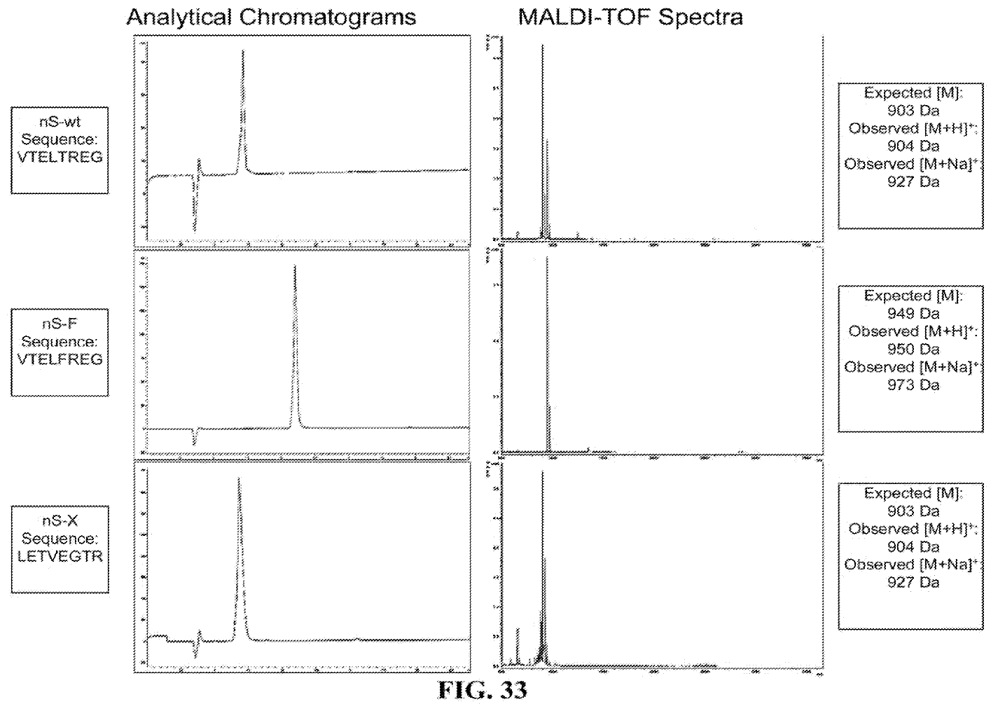

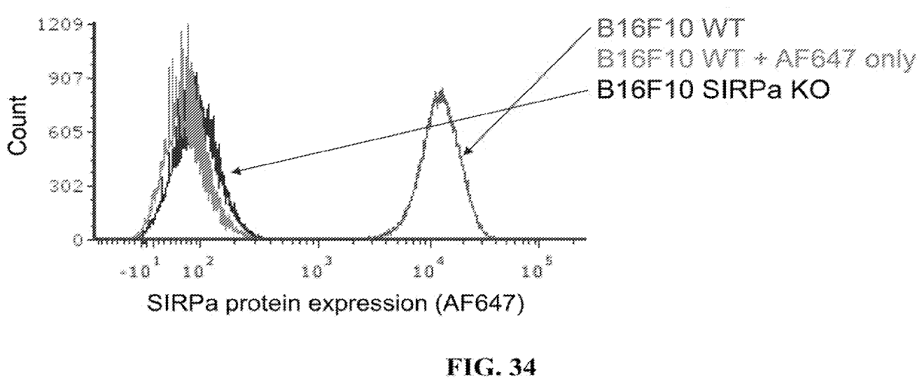

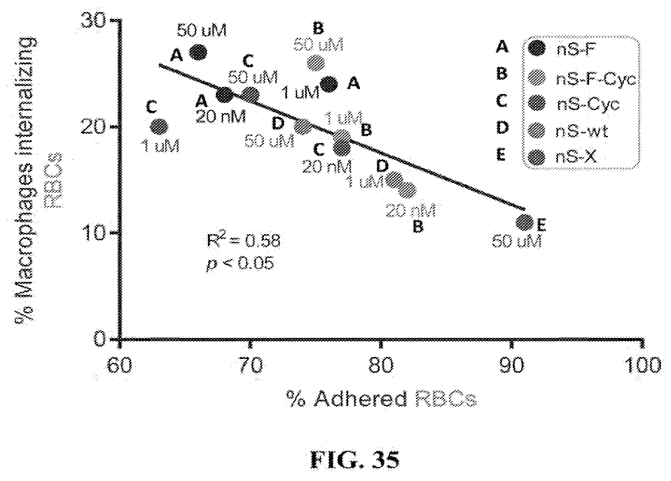

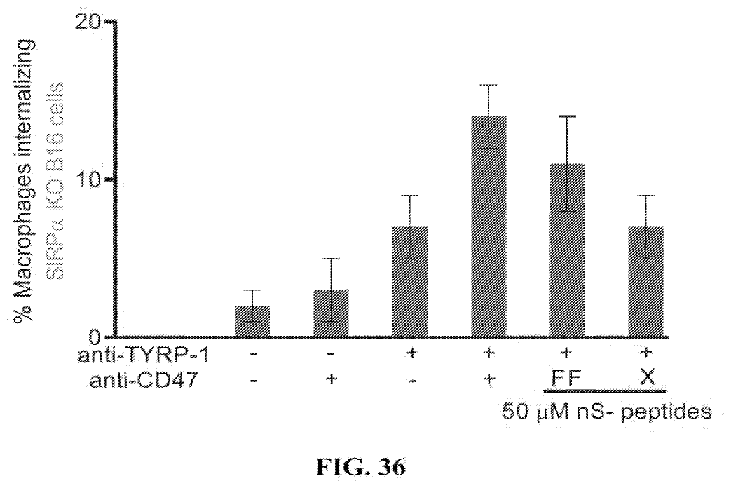

To focus on the possible function of the nS peptides as soluble antagonists of CD47-SIRPα, their effects were first tested on phagocytosis of mouse melanoma B16F10 cells (B16 for brevity) by mouse bone marrow derived macrophage (BMDM). B16s are a standard model for preclinical immunotherapy, but success with anti-CD47 blockade remains a challenge even when combined with IgG-opsonization of B16 to promote phagocytosis. As per the latter study, B16 opsonization was done here with monoclonal anti-TYRP1 IgG against the melanocyte-specific tyrosinase-related protein 1 (TYRP1) for the phagocytosis assay. B16 cells (expressing GFP) were detached from their culture dishes, opsonized with anti-TYRP1, and then added to well-spread BMDMs that were labeled with red dye and pre-treated or not with the various peptides (FIG. 29A, top). Microscopy showed internalization, or “eating,” of rounded B16s especially when anti-mCD47 or the various nS peptides were also added (FIG. 29A, images). Scrambled nS-X peptide was the clear exception. B16s that were not engulfed were often attached and spread on the dishes similar to the BMDMs.

Quantitation of the amount of macrophages that had engulfed opsonized B16s was done for WT B16s as well as SIRPα knockout B16s (KO). Many cell and cancer types express SIRPα, including B16 melanoma (FIG. 34). KO of SIRPα on B16s removes its cis interactions with CD47 on the same cell, allowing for more accessible “don't eat me” signaling by CD47 through trans interactions with SIRPα on the opposing macrophage. This effectively represents cancers that are CD47-rich and SIRPα-negative.

Anti-mCD47 on both B16 lines showed baseline eating (FIG. 29B: (−,−) vs (−,+)), whereas anti-TYRPI consistently showed higher opsonized eating that increased further upon addition of anti-mCD47 (FIG. 29B: (+,-) vs (+,+)). The latter inhibition of CD47 gave similar levels of eating for WT and KO B16s, consistent with equal inhibition of CD47′s “Self” interactions in trans with SIRPα on macrophages. However, the results also represent 50% and 100% respective increases relative to anti-TYRPI opsonization alone, consistent with the model that the KO B16 effectively displays more CD47.

Maximum levels of phagocytic macrophages were measured with nS-F, nS-F-Cyc, and nS-Cyc peptides (FIG. 29B: (+,nS peptides)), with lower levels for linear nS-wt that are similar to the effects of anti-mCD47 on engulfment of opsonized WT and KO B16s. Scrambled nS-X peptide does not affect opsonization-driven eating of the two B16 lines. The maximum levels of eating with soluble nS-F, nS-F-Cyc, and nS-Cyc are consistent with disruption of both trans and cis interactions between CD47—SIRPα on the macrophages, per previous studies showing suppression of a basal level of inhibitory “don't eat me” signal; nS peptides are unlikely to bind and inhibit SIRPα on WT B16 cells because excess peptide is washed away in the assay before adding B16s to the macrophages. Regardless, the increased phagocytosis follows the trend cyclic >wildtype >scrambled activity, and while the mutant Phe residue makes linear >wildtype, it has no effect on the cyclic.

The above phagocytosis results with mouse cells are somewhat surprising because the nS peptides of the present disclosure were designed based on the structure of human CD47 interacting with human SIRPα (FIGS. 28A,B), and yet the mouse sequences generally differ. Phagocytosis assays therefore focused next on a simple human system of fresh human red blood cells (RBCs) and human THP-1 macrophages, as previously studied. RBCs were opsonized with a polyclonal IgG (a rabbit anti-human RBC) and then added to the cultured macrophages that had been nS peptide treated (FIG. 29C-Top). RBCs were the cells with which CD47′s ‘Marker of Self’ function was first identified, but solely blocking CD47 on the RBC does not significantly affect baseline eating, with<5% of macrophages internalizing one or more RBCs (FIG. 29D: (−,−) vs (−,+)). Opsonization of RBCs more than doubles the extent of phagocytosis and addition of anti-CD47 doubles again this internalization (FIG. 29D: (−,+) vs (+,+)). The results are consistent with ‘self’ recognition only when coupled to a pro-phagocytic cue.

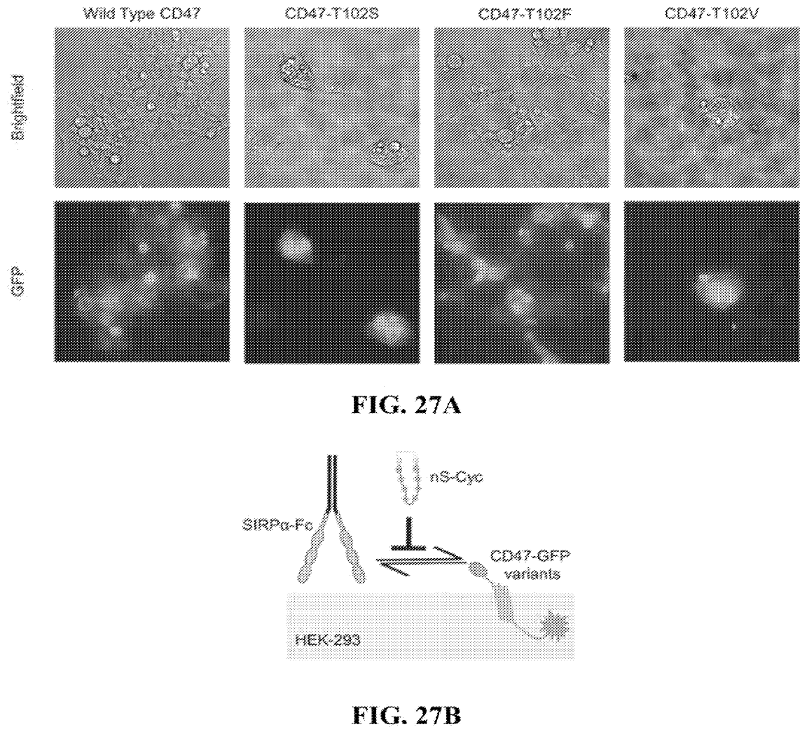

Similar studies also assessed the phagocytosis of IgG-opsonized human red blood cells (RBCs) by incubating solutions of nS-Cyc with adherent human and mouse macrophages (FIG. 27A: Left Panel). Internalization of opsonized RBCs after treatment of both human and mouse macrophages with nS-Cyc was observed (FIG. 27A: Images) at slightly higher levels than opsonized and anti-CD47 blocked RBCs (FIG. 27B). nS-Cyc phagocytic activity in mouse macrophages at saturating concentrations is weaker than in human, consistent with nS-wt activity (FIG. 27C). However, nS-Cyc enhanced human and mouse macrophage internalization more than the linear nS-wt, supporting an advantage of using cyclic peptides. nS-F potency on both human and mouse macrophages is significantly greater than any other peptide tested, indicating that sequence is an important determining factor of efficacy.

Maximum levels of phagocytic macrophages were once again measured with peptides nS-F, nS-F-Cyc, and nS-Cyc peptides (FIG. 29B: (+,nS peptides)), which aligns very well with the mouse results. Lower levels of phagocytosis for linear nS-wt are likewise similar to anti-CD47 effects on engulfment of opsonized RBCs, and scrambled nS-X peptide does not affect opsonization-driven eating of RBCs. The maximum levels of eating with soluble nS-F, nS-F-Cyc, and nS-Cyc are again consistent with disruption of both trans and cis interactions between CD47—SIRPα on the macrophages. Overall, mouse and human assay results are not only similar in trend but also similar in quantitation with the nS-peptide maxima of ˜25% for phagocytosis of opsonized cells.

### Example 11: Maximum Phagocytosis also Minimizes Residual Adhesion to Macrophages

Opsonized RBCs that contact macrophages will tend to adhere to the macrophages, and any subsequent internalization should in principle lead to a decrease in the number of macrophage-adherent RBCs. Importantly, the CD47—SIRPα interaction mediates RBC adhesion, and a peptide such as nS-X that does not seem to affect the CD47—SIRPα interaction (FIGS. 29B, D) should lead to more residual adhesion than, for example, the nS-Cyc peptide that effectively inhibits CD47—SIRPα while increasing internalization (FIG. 30A, sketch).

To measure the residual adhesion of RBCs and distinguish it from internalization by macrophages, the engulfment assay with human RBCs used a secondary antibody against the opsonizing IgG, which allowed us to clearly see RBCs in contact with macrophages that are not internalized (FIG. 29C). The cells are also firmly attached to the macrophages because the culture dishes were rinsed after fixation and secondary antibody labeling. Quantitation of the macrophage-adherent RBCs shows a significant anti-correlation with phagocytosis for the nS peptide samples (p<0.05) (FIGS. 30A plot, 35). All of the RBCs in these samples are equally opsonized with IgG that binds Fc-receptors (FcR's) on macrophages, and so differences in measured adhesion implicate differences in the CD47—SIRPα interaction. Blockade of this immune checkpoint interaction by nS-F and nS-Cyc understandably shows the lowest amount of residual adhesion to macrophages, whereas the inactive nS-X has no effect on phagocytosis of opsonized RBCs (FIG. 29D), resulting in predominantly adherent RBCs.

When a melanoma cell suspension is added to a macrophage culture, the melanoma cell might (i) adhere and spread on the plastic culture dish far from a macrophage, or (ii) adhere entirely or partially to a macrophage, or else (iii) adhere to a macrophage and be internalized (FIG. 29A, images). Note that B16′s are typical of adherent cell types and will attach strongly to plastic within 1-2 hrs, unlike RBCs; likewise in vivo, B16′s will generally adhere to each other and to other cells and matrix, unlike RBCs in freely flowing blood. Opsonized melanoma cells should nonetheless adhere to any contacting macrophages through both the IgG-FcR interaction and the CD47—SIRPα interaction, although the latter should again be inhibited by the nS peptides that specifically antagonize CD47—SIRPα (FIG. 30B, sketch). Based on these observations, the many opsonized B16s that were not internalized but were in clear contact with a macrophage were defined as macrophage-adherent while also possibly adhering to the plastic dish (FIG. 30B, inset image); quantitation of these macrophage-adhered B16s again shows an anti-correlation with phagocytosis for the numerous nS peptide samples (p<0.05) (FIG. 30B, plot). The nS peptides that maximized phagocytosis also minimized the macrophage-adhered B16s, consistent with the human RBC results despite the additional means of B16 attachment in these assays. Moreover, the WT B16s adhered less than the corresponding KO B16s, consistent with the greater phagocytosis of the WT B16s.

These human and mouse plots were fit with a suppression model:

y=[yₘₐₓ−(A/B)^{m}]+A^{m}/(B^{m}+x^{m})

Key fit parameters are remarkably similar for both species, with yₘₐₓ˜30%, m˜5, and only ˜two-fold differences in A^{m} and B^{m} parameters. The finding of a common phagocytosis asymptote yₘₐₓ indicates that for this assay done over a few hours the macrophage cultures are saturating their phagocytic capacity to a similar extent.

### Example 12: nS Peptides have Stronger Effects With human than with Mouse Macrophages

All of the results above used 50 μM peptide, which is >50-fold higher than the ˜0.1-1 μM affinity between human CD47 and human SIRPα. Using lower concentrations in the assays of opsonized human-RBCs being eaten by human macrophages not only shows that 50 μM has a saturating effect for all of the active peptides (FIG. 31A) but also that the effective potency follows trends nS-Cyc˜nS-F-Cyc˜nS-F >nS-wt >nS-X. The effective potency (k_{eff}) of ˜1 μM for nS-wt is weaker than the affinity of CD47—SIRPα, and could reflect the fact that this short peptide lacks the proper loop structure for binding (entropy contribution) and also lacks other contacts that full-length CD47 has with SIRPα (enthalpic contribution). The finding of k_{eff}˜10 nM for the cyclic peptides indeed suggests some advantages of a loop conformation. The Phe substitution also adds to the affinity of the linear nS-F relative to nS-wt, but evidently does not help or hinder the cyclic peptide. The lower concentrations of active peptides also typically show more RBC adhesion than the saturating 50 μM (FIG. 35), consistent with the expected trend (FIG. 30A). Surprisingly, similar dilution studies of the active nS peptides show no activity in the mouse-focused assays of opsonized B16s being eaten by primary mouse macrophages - for both WT and KO B16s (FIG. 31B). These human-derived sequences are estimated to have much weaker effective activities (keff˜20-120 μM) as antagonists of mouse CD47—SIRPα interactions that enhance mouse cell phagocytosis.

### Example 13: Selected Discussion

The nano—Self sequence nS-wt is directly from the human CD47 loop that interacts with human SIRPα (FIG. 28A) and differs from mouse CD47 only at the central Thr residue, which is a Ser in mouse. This conservative Thr→Ser difference applies to the C57 strain of mouse, from which the B16 melanoma cells and macrophages studied here were obtained. It also applies to the BALB/c strain of mouse, for which recent studies have recently shown the linear nS-F peptide is antagonistic toward ‘self’ recognition by J774A. 1 macrophages. Antagonistic activity in the mouse melanoma assays (FIGS. 29B, 30B) by the human wildtype cyclic and linear peptides is thus anticipated.

Weak effective affinity of all nS peptides in the mouse assays (FIG. 31B) is consistent with other studies of human CD47 interacting with various mouse SIRPα. Two of SIRPα's three binding loops and several contact residues show sequence differences (FIG. 30C). One notable difference is a hydrophobic Phe in a mouse loop of SIRPα, which in human is a non-conserved Gln and which will tend to interact with the Phe in the nS-F and nS-F-Cyc peptides, thereby competing with hydrophobic interactions deeper in the binding pocket. Indeed, recent studies of a bivalent nS-FF antagonist greatly increased human macrophage phagocytosis of human erythroleukemia and RBCs, but this bivalent shows reduced activity with mouse macrophages (FIG. 36). Sequences and structures of both SIRPα and CD47 are likely to affect binding and activity, consistent with recent analyses of antibodies. Furthermore, outside the binding site, the FG loops of SIRPα in C57 and BALB/c show Ser-102 and Glu-103 are absent in SIRPα of both human and NOD mouse strain that binds human CD47 (FIG. 31C), and inserting these two amino acids into NOD SIRPα has been shown to weaken human CD47 binding.

Although there are many FDA-approved therapeutic antibodies against various diseases and malignancies, many clinical challenges associated with large IgG antibodies persist, including immunogenicity, limited tissue penetration, on-target toxicities, and production costs. Naturally derived and synthetic peptides have potential clinical advantages such as high tissue penetration, high specificity for a target, and minimal accumulation in organs, but challenges include short plasma half-life, low bioavailability, and poor pharmacokinetics. Nevertheless, peptide therapeutics are increasingly being approved: among the 17 FDA-approved peptides since 2016, 11 of these peptides are between 3 and 8 amino acids in length, and five are cyclic disulfide peptides. Natural cyclic peptides resist proteolytic degradation and show high activity, which generally supports the pharmaceutical development of cyclic peptides. For CD47—SIRPα, peptides that include large macrocycles have been made to bind CD47 and have shown some anti-tumor efficacy in mouse models. The nS peptides are the first to directly target and antagonize SIRPα. SIRPα is more restricted in its expression compared to CD47, which is expressed on every cell in the body, and so CD47-binding peptides that are systemically infused will first bind RBCs (favoring phagocytosis) and then bind all accessible tissues.

Soluble, recombinant, and mutant CD47 and SIRPα proteins can block the macrophage checkpoint as can antibodies even if they lack the Fc domain. Such antagonists have been pursued to minimize IgG opsonization of targets that favors FcR-mediated phagocytosis and likely explains the various forms of anemia that result from anti-CD47 infusions in the clinic. The nS peptides potently and consistently enhanced phagocytosis of opsonized and mouse cancer cells, and they do so as effectively as anti-CD47 inhibition on these target cells. Peptides lack Fc domains, and so phagocytosis is driven solely by the specificity of IgG opsonization.

### Example 14: Conclusion

Cyclic nS peptides are potent SIRPα antagonists derived from the CD47 binding loop. The disulfide bridge in nS-F-Cyc is expected to enhance the peptide half-life in serum, but other disulfide mimetics and cyclization methods can be pursued to further stabilize the structure. Substitution of the critical, polar Thr to hydrophobic Phe resulted with enhanced potency in both linear and cyclic peptides, demonstrating the significance of the sequence to the mechanism of SIRPα antagonism. Moreover, the weaker peptide activity in mouse macrophages relative to human and the opposite effects of bivalent nS-FF in human and mouse macrophages further underscore sequence complementarity as essential for potent activity.

### Enumerated Embodiments

The following enumerated embodiments are provided, the numbering of which is not to be construed as designating levels of importance.

Embodiment 1 provides a compound of Formula (I), or a salt or solvate thereof:

(BINDERᵢ-LINKERᵢ)ᵢCORE (I)

wherein:
each occurrence of BINDERᵢ is independently a P1 peptide, which comprises the amino acid sequence of SEQ ID NO:1, wherein Xaa5 is Thr or a natural or synthetic amino acid that is not Thr:

Val Thr Glu Leu Xaa5 Arg Glu Gly (SEQ ID NO:1)
each occurrence of LINKERᵢ is independently a linker;
CORE is a moiety independently covalently bound to each (BINDERᵢ-LINKERᵢ) through LINKERᵢ;
‘i’ is an integer equal to or greater than 2.

Embodiment 2 provides the compound of Embodiment 1, wherein ‘i’ is 2, 3, 4, 5, 6, 7, 8, 9, 10, 11, or 12.

Embodiment 3 provides the compound of any of Embodiments 1-2, wherein ‘i’ is 2 and the compound, or a salt or solvate thereof, is: (BINDER1-LINKER1)-CORE-(LINKER2-BINDER2) (I-1).

Embodiment 4 provides the compound of any of Embodiments 1-3, wherein ‘i’ is 3 and the compound, or a salt or solvate thereof, is:

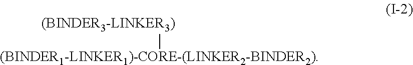

Embodiment 5 provides the compound of any of Embodiments 1-4, wherein ‘i’ is 4 and the compound, or a salt or solvate thereof, is:

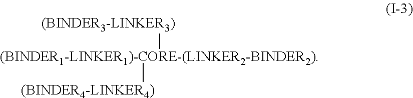

Embodiment 6 provides the compound of any of Embodiments 1-5, wherein Xaa5 is less polar than Thr.

Embodiment 7 provides the compound of any of Embodiments 1-6, wherein Xaa5 is Thr, Ser, Phe, Val, Ala, Leu, Ile, Pro, or Met.

Embodiment 8 provides the compound of any of Embodiments 1-7, wherein Xaa5 is not Thr.

Embodiment 9 provides the compound of any of Embodiments 1-8, wherein in P1 the N-terminus of the amino sequence of SEQ ID NO: 1 is directly coupled with the C-terminus of an amino acid or peptide that is not Gly-Asn-Tyr-Thr-Cys-Glu, Asn-Tyr-Thr-Cys-Glu, Tyr-Thr-Cys-Glu, Thr-Cys-Glu, Cys-Glu, Thr-Glu, or Glu.

Embodiment 10 provides the compound of any of Embodiments 1-9, wherein in Pl the

C-terminus of the amino sequence of SEQ ID NO: 1 is directly coupled with the N-terminus of an amino acid or peptide that is not Glu-Thr-Ile-Ile-Glu, Glu-Thr-Ile-Ile, Glu-Thr-Ile, Glu-Thr, Glu-Cys, or Glu.

Embodiment 11 provides the compound of any of Embodiments 1-10, wherein at least one BINDERᵢ comprises the amino acid sequence of SEQ ID NOs: 2-4.

Embodiment 12 provides the compound of any of Embodiments 1-11, wherein at least one BINDERᵢ comprises the amino acid sequence of SEQ ID NOs: 6-8.

Embodiment 13 provides the compound of any of Embodiments 1-12, wherein each LINKERᵢ independently comprises a group of Formula (II):

*-(CHR′)m1-X1-(CHR′-CHR′-X2)m2-(CHR′)m3-X3- (II)

wherein:
* indicates the bond between the LINKERᵢ and the BINDERi;
m1, m2, and m3 are independently an integer ranging from 0-100;
each occurrence of X¹ and X² are independently selected from the group consisting of absent (a bond), O, and N(R′);
X³ forms a covalent bond to the CORE and is selected from the group consisting of absent (a bond), C(═O), O, S, and N(R′);
each occurrence of R′is independently selected from the group consisting of hydrogen, optionally substituted C₁-C₆ alkyl, optionally substituted C₃-C₈ cycloalkyl, and optionally substituted C₃-C₈ cycloheteroalkyl.

Embodiment 14 provides the compound of any of Embodiments 1-13, wherein each LINKERᵢ is independently about 5 Å, 6 Å, 7 Å, 8 Å, 9 Å, 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, or 25 Å in length.

Embodiment 15 provides the compound of any of Embodiments 1-14, wherein the CORE is a chemical moiety comprising ‘i’ groups, wherein each group is covalently linked to an individual LINKERᵢ, and wherein each group is independently a carboxylic acid, a primary or secondary amine, a hydroxyl group, a thiol group, or an alkene group.

Embodiment 16 provides the compound of any of Embodiments 1-15, wherein the CORE is an amino acid or a (poly)peptide.

Embodiment 17 provides the compound of any of Embodiments 1-16, wherein the CORE is an amino acid or (poly)peptide comprising at least one of Asp, Glu, Lys, Arg, Ser, Thr, Orn, and Cys.

Embodiment 18 provides the compound of any of Embodiments 1-17, which is nS-FF, nS-VV, or nS-F4.

Embodiment 19 provides a pharmaceutical composition comprising the compound of any of Embodiments 1-18 and at least one pharmaceutically acceptable carrier.

Embodiment 20 provides a method of inhibiting biological activity of a signal regulatory protein alpha (SIRPα), the method comprising contacting the SIRPα with the compound of any of Embodiments 1-18.

Embodiment 21 provides the method of Embodiment 20, wherein the SIRPα is expressed on the surface of a macrophage.

Embodiment 22 provides the method of Embodiment 21, wherein the macrophage is in vivo in a mammal.

Embodiment 23 provides a method of enhancing phagocytosis of a cell by a macrophage, the method comprising contacting the macrophage with the compound of any of Embodiments 1-18.

Embodiment 24 provides the method of Embodiment 23, wherein the contacting inhibits at least in part the biological activity of a signal regulatory protein alpha (SIRPα) expressed on the surface of the macrophage.

Embodiment 25 provides the method of Embodiment 23, wherein the contacting takes place in the vicinity of a cell to be phagocytized by the macrophage.

Embodiment 26 provides the method of Embodiment 25, wherein the cell is at least partially coated by opsonizing antibodies.

Embodiment 27 provides the method of Embodiment 25, wherein the cell to be phagocytized is cancerous.

Embodiment 28 provides the method of any of Embodiments 23-27, which is performed in vivo in a subject suffering from cancer.

Embodiment 29 provides the method of Embodiment 28, wherein the compound is administered systemically to the subject.

Embodiment 30 provides the method of Embodiment 28, wherein the compound is administered to the vicinity of the cancer and/or intratumorally in the subject.

Embodiment 31 provides the method of any of Embodiments 28-30, wherein the subject is human.

Embodiment 32 provides a compound of Formula (I), or a salt or solvate thereof:

(BINDERᵢ- LINKERᵢ)ᵢCORE (I)

wherein:
each occurrence of BINDERᵢ is independently a P1 peptide, wherein P1 comprises Cysª—SEQ ID NO:1-Cysb, wherein Cysª and Cysb are bridged by a disulfide bond resulting in a cyclic peptide and wherein Xaa5 is Thr or a natural or synthetic amino acid that is not Thr;

Val Thr Glu Leu Xaa5 Arg Glu Gly (SEQ ID NO:1)
each occurrence of LINKERᵢ is independently a linker;
CORE is a moiety independently covalently bound to each (BINDERᵢ-LINKERᵢ) through LINKERᵢ;
‘i’ is an integer equal to or greater than 2;

Embodiment 33 provides the compound of Embodiment 32, wherein ‘i’ is 2, 3, 4, 5, 6, 7, 8, 9, 10, 11, or 12.

Embodiment 34 provides the compound of any of Embodiments 32-33, wherein ‘i’ is 2 and the compound, or a salt or solvate thereof, is:

(BINDER1-LINKER1)-CORE-(LINKER2-BINDER2) (I-1).

Embodiment 35 provides the compound of any of Embodiments 32-34, wherein ‘i’ is 3 and the compound, or a salt or solvate thereof, is:

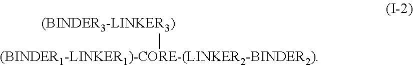

Embodiment 36 provides the compound of any of Embodiments 32-35, wherein ‘i’ is 4 and the compound, or a salt or solvate thereof, is:

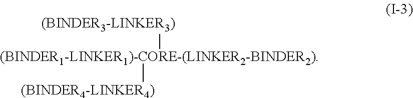

Embodiment 37 provides the compound of any of Embodiments 32-36, wherein Xaa5 is less polar than Thr.

Embodiment 38 provides the compound of any of Embodiments 32-37, wherein Xaa5 is Thr, Ser, Phe, Val, Ala, Leu, Ile, Pro, or Met.

Embodiment 39 provides the compound of any of Embodiments 32-38, wherein Xaa5 is not Thr.

Embodiment 40 provides the compound of any of Embodiments 32-39, wherein in P1 the N-terminus of the amino sequence of SEQ ID NO:1 is directly coupled with the C-terminus of an amino acid or peptide that is not Gly-Asn-Tyr-Thr-Cys-Glu, Asn-Tyr-Thr-Cys-Glu, Tyr-Thr-Cys-Glu, Thr-Cys-Glu, Cys-Glu, Thr-Glu, or Glu.

Embodiment 41 provides the compound of any of Embodiments 32-40, wherein in P1 the C-terminus of the amino sequence of SEQ ID NO: 1 is directly coupled with the N-terminus of an amino acid or peptide that is not Glu-Thr-Ile-Ile-Glu, Glu-Thr-Ile-Ile, Glu-Thr-Ile, Glu-Thr, Glu-Cys, or Glu.

Embodiment 42 provides the compound of any of Embodiments 32-41, wherein at least one BINDERᵢ comprises the amino acid sequence of SEQ ID NOs: 2-4.

Embodiment 43 provides the compound of Embodiments 32-42, wherein at least one BINDERᵢ comprises the amino acid sequence of SEQ ID NOs: 6-8.

Embodiment 44 provides the compound of any of Embodiments 32-43, wherein each LINKERᵢ independently comprises a group of Formula (II):

*-(CHR′)ₘ₁-X¹-(CHR′-CHR′-X²)ₘ₂-(CHR′)ₘ₃-X³- (II)

wherein:
* indicates the bond between the LINKERᵢ and the BINDERᵢ;
m1, m2, and m3 are independently an integer ranging from 0-100;
each occurrence of X¹ and X² are independently selected from the group consisting of absent (a bond), O, and N(R′);
X³ forms a covalent bond to the CORE and is selected from the group consisting of absent (a bond), C(═O), O, S, and N(R′);
each occurrence of R′is independently selected from the group consisting of hydrogen, optionally substituted C₁-C₆ alkyl, optionally substituted C₃-C₈ cycloalkyl, and optionally substituted C₃-C₈ cycloheteroalkyl.

Embodiment 45 provides the compound of any of Embodiments 32-44, wherein each LINKERᵢ is independently about 5 Å, 6 Å, 7 Å, 8 Å, 9 Å, 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, or 25 Å in length.

Embodiment 46 provides the compound of any of Embodiments 32-45, wherein the CORE is a chemical moiety comprising ‘i’ groups, wherein each group is covalently linked to an individual LINKERᵢ, and wherein each group is independently a carboxylic acid, a primary or secondary amine, a hydroxyl group, a thiol group, or an alkene group.

Embodiment 47 provides the compound of any of Embodiments 32-46, wherein the CORE is an amino acid or a (poly)peptide.

Embodiment 48 provides the compound of any of Embodiments 32-46, wherein the CORE is an amino acid or (poly)peptide comprising at least one of Asp, Glu, Lys, Arg, Ser, Thr, Orn, and Cys.

Embodiment 49 provides the compound of any of Embodiments 32-48, which is nS-FF, nS-VV, or nS-F4.

Embodiment 50 provides a pharmaceutical composition comprising the compound of any of Embodiments 32-49 and at least one pharmaceutically acceptable carrier.

Embodiment 51 provides a method of inhibiting biological activity of a signal regulatory protein alpha (SIRPα) , the method comprising contacting the SIRPα with the compound of any of Embodiments 32-49.

Embodiment 52 provides the method of Embodiment 51, wherein the SIRPα is expressed on the surface of a macrophage.

Embodiment 53 provides the method of Embodiment 52, wherein the macrophage is in vivo in a mammal.

Embodiment 54 provides a method of enhancing phagocytosis by a macrophage, the method comprising contacting the macrophage with the compound of any of Embodiments 32-49.

Embodiment 55 provides the method of Embodiment 54, wherein the contacting inhibits at least in part the biological activity of a signal regulatory protein alpha (SIRPα) expressed on the surface of the macrophage.

Embodiment 56 provides the method of Embodiment 54, wherein the contacting takes place in the vicinity of a cell to be phagocytized by the macrophage.

Embodiment 57 provides the method of Embodiment 56, wherein the cell is at least partially coated by opsonizing antibodies.

Embodiment 58 provides the method of Embodiment 56, wherein the cell to be phagocytized is cancerous.

Embodiment 59 provides the method of any of Embodiments 51-58, which is performed in vivo in a subject suffering from cancer.

Embodiment 60 provides the method of Embodiment 59, wherein the compound is administered systemically to the subject.

Embodiment 61 provides the method of Embodiment 59, wherein the compound is administered to the vicinity of the cancer and/or intratumorally in the subject.

Embodiment 62 provides the method of any of Embodiments 59-61, wherein the subject is human.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A compound of Formula (I), or a salt or solvate thereof:
(BINDERᵢ-LINKERᵢ)ᵢCORE (I)
wherein:
each occurrence of BINDERᵢ is independently a PR peptide, which comprises the amino acid sequence of SEQ ID NO:1, wherein Xaa5 is Thr or a natural or synthetic amino acid that is not Thr:
Val Thr Glu Leu Xaa5 Arg Glu Gly (SEQ ID NO:1)
each occurrence of LINKERᵢ is independently a linker;
CORE is a moiety independently covalently bound to each (BINDERi-LINKERi) through LINKERᵢ;
‘i’ is an integer equal to or greater than 2.

2. The compound of claim 1, wherein ‘i’ is 2, 3, 4, 5, 6, 7, 8, 9, 10, 11, or 12.

3. The compound of claim 1, wherein one of the following applies:
(a) ‘i’ is 2 and the compound, or a salt or solvate thereof, is:
(BINDER1-LINKER1)-CORE-(LINKER2-BINDER2) (I-1);
(b) ‘i’ is 3 and the compound, or a salt or solvate thereof, is: 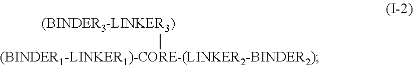
(c) ‘i’ is 4 and the compound, or a salt or solvate thereof, is :.

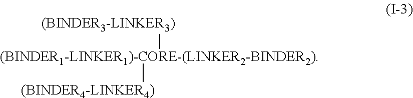

4-5. (canceled)

6. The compound of claim 1, wherein Xaa5 is less polar than Thr or wherein Xaa5 is not Thr.

7. The compound of claim 1, wherein Xaa5 is Thr, Ser, Phe, Val, Ala, Leu, Ile, Pro, or Met.

8. (canceled)

9. The compound of claim 1, wherein at least one of the following applies:
(a) in P1 the N-terminus of the amino sequence of SEQ ID NO: 1 is directly coupled with the C-terminus of an amino acid or peptide that is not Gly-Asn-Tyr-Thr-Cys-Glu, Asn-Tyr-Thr-Cys-Glu, Tyr-Thr-Cys-Glu, Thr-Cys-Glu, Cys-Glu, Thr-Glu, or Glu;
(b) in Pl the C-terminus of the amino sequence of SEQ ID NO: 1 is directly coupled with the N-terminus of an amino acid or peptide that is not Glu-Thr-Ile-Ile-Glu, Glu-Thr-Ile-Ile, Glu-Thr-Ile, Glu-Thr, Glu-Cys, or Glu.

10. (canceled)

11. The compound of claim 1, wherein at least one BINDERᵢ comprises the amino acid sequence selected from the group consisting of SEQ ID NOs: 2-4 and 6-8.

12. (canceled)

13. The compound of claim 1, wherein each LINKERᵢ independently comprises a group of Formula (II):
*-(CHR′)m1-X1-(CHR′-CHR′-X2)m2-(CHR′)m3-X3- (II)
wherein:
* indicates the bond between the LINKERᵢ and the BINDERi;
m1, m2, and m3 are independently an integer ranging from 0-100;
each occurrence of X¹ and X² are independently selected from the group consisting of absent (a bond), O, and N(R′);
X³ forms a covalent bond to the CORE and is selected from the group consisting of absent (a bond), C(═O), O, S, and N(R′);
each occurrence of R′is independently selected from the group consisting of hydrogen, optionally substituted C₁-C₆ alkyl, optionally substituted C₃-C₈ cycloalkyl, and optionally substituted C₃-C₈ cycloheteroalkyl.

14. The compound of claim 1, wherein at least one of the following applies:
(a) each LINKERᵢ is independently about 5 Å, 6 Å, 7 Å, 8 Å, 9 Å, 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, or 25 Å in length;
(b) the CORE is a chemical moiety comprising ‘i’ groups, wherein each group is covalently linked to an individual LINKERᵢ, and wherein each group is independently a carboxylic acid, a primary or secondary amine, a hydroxyl group, a thiol group, or an alkene group;
(c) the CORE is an amino acid or a (poly)peptide, optionally comprising at least one of Asp, Glu, Lys, Arg, Ser, Thr, Orn, and Cys;
(d) the compound is nS-FF, nS-VV, or nS-F4.

15-18. (canceled)

19. A pharmaceutical composition comprising the compound of claim 1 and at least one pharmaceutically acceptable carrier.

20. A method of inhibiting biological activity of a signal regulatory protein alpha (SIRPα), the method comprising contacting the SIRPα with the compound of claim 1.

21. The method of claim 20, wherein the SIRPα is expressed on the surface of a macrophage, optionally wherein the macrophage is in vivo in a mammal.

22. (canceled)

23. A method of enhancing phagocytosis of a cell by a macrophage, the method comprising contacting the macrophage with the compound of claim 1.

24. The method of claim 23, wherein at least one of the following applies:
(a) the contacting inhibits at least in part the biological activity of a signal regulatory protein alpha (SIRPα) expressed on the surface of the macrophage;
(b) the contacting takes place in the vicinity of the cell to be phagocytized by the macrophage;
(c) the cell is at least partially coated by opsonizing antibodies;
(d) the cell to be phagocytized is cancerous;
(e) the contacting is performed in vivo in a subject suffering from cancer, optionally wherein the compound is administered systemically to the subject or wherein the compound is administered to the vicinity of the cancer or intratumorally in the subject., optionally wherein the subject is human.

25-31. (canceled)

32. A compound of Formula (I), or a salt or solvate thereof:
(BINDERᵢ-LINKERᵢ)ᵢCORE (I)
wherein:
each occurrence of BINDERᵢ is independently a P1 peptide, wherein P1 comprises Cysª-SEQ ID NO:1-Cysb, wherein Cysª and Cysb are bridged by a disulfide bond resulting in a cyclic peptide and wherein Xaa5 is Thr or a natural or synthetic amino acid that is not Thr;
Val Thr Glu Leu Xaa5 Arg Glu Gly (SEQ ID NO:1)
each occurrence of LINKERᵢ is independently a linker;
CORE is a moiety independently covalently bound to each (BINDERᵢ-LINKERᵢ) through LINKERᵢ;
‘i’ is an integer equal to or greater than 2;

33. The compound of claim 32, wherein ‘i’ is 2, 3, 4, 5, 6, 7, 8, 9, 10, 11, or 12.

34. The compound of claim 32, wherein at least one of the following applies:
(a) ‘i’ is 2 and the compound, or a salt or solvate thereof, is:
(BINDER1-LINKER1)-CORE-(LINKER2-BINDER2) (I-1);
(b) ‘i’ is 3 and the compound, or a salt or solvate thereof, is: 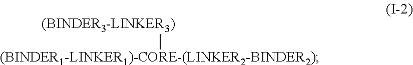
(c) ‘i’ is 4 and the compound, or a salt or solvate thereof, is:

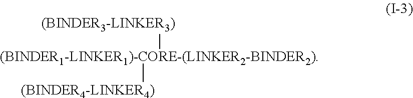

3-36. (canceled)

37. The compound of claim 32, wherein Xaa5 is less polar than Thr or wherein Xaa5 is not Thr.

38. The compound of claim 32, wherein Xaa5 is Thr, Ser, Phe, Val, Ala, Leu, Ile, Pro, or Met.

39. (canceled)

40. The compound of claim 32, wherein at least one of the following applies:
(a) in P1 the N-terminus of the amino sequence of SEQ ID NO: 1 is directly coupled with the C-terminus of an amino acid or peptide that is not Gly-Asn-Tyr-Thr-Cys-Glu, Asn-Tyr-Thr-Cys-Glu, Tyr-Thr-Cys-Glu, Thr-Cys-Glu, Cys-Glu, Thr-Glu, or Glu;
in Pl the C-terminus of the amino sequence of SEQ ID NO: 1 is directly coupled with the N-terminus of an amino acid or peptide that is not Glu-Thr-Ile-Ile-Glu, Glu-Thr-Ile-Ile, Glu-Thr-Ile, Glu-Thr, Glu-Cys, or Glu (b)

41. (canceled)

42. The compound of claim 32, wherein at least one BINDERᵢ comprises the amino acid sequence selected from the group consisting of SEQ ID NOs: 2 and 6-8.

43. (canceled)

44. The compound of claim 32, wherein each LINKERᵢ independently comprises a group of Formula (II):
*-(CHR′)ₘ₁-X¹-(CHR′-CHR′-X²)ₘ₂-(CHR′)ₘ₃-X³- (II)
wherein:
* indicates the bond between the LINKERᵢ and the BINDERi;
m1, m2, and m3 are independently an integer ranging from 0-100;
each occurrence of X¹ and X² are independently selected from the group consisting of absent (a bond), O, and N(R′);
X³ forms a covalent bond to the CORE and is selected from the group consisting of absent (a bond), C(═O), O, S, and N(R′);
each occurrence of R′is independently selected from the group consisting of hydrogen, optionally substituted C₁-C₆ alkyl, optionally substituted C₃-C₈ cycloalkyl, and optionally substituted C₃-C₈ cycloheteroalkyl.

45. The compound of claim 32, wherein at least one of the following applies:
(a) each LINKERᵢ is independently about 5 Å, 6 Å, 7 Å, 8 Å, 9 Å, 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, or 25 Å in length;
(b) the CORE is a chemical moiety comprising ‘i’ groups, wherein each group is covalently linked to an individual LINKERᵢ, and wherein each group is independently a carboxylic acid, a primary or secondary amine, a hydroxyl group, a thiol group, or an alkene group;
(c) the CORE is an amino acid or a (poly)peptide, optionally comprising at least one of Asp, Glu, Lys, Arg, Ser, Thr, Orn, and Cys;
(d) the compound is nS-FF, nS-VV, or nS-F4

46-49. (canceled)

50. A pharmaceutical composition comprising the compound of claim 32 and at least one pharmaceutically acceptable carrier.

51. A method of inhibiting biological activity of a signal regulatory protein alpha (SIRPα) , the method comprising contacting the SIRPα with the compound of claim 32.

52. The method of claim 51, wherein the SIRPα is expressed on the surface of a macrophage, which optionally is in vivo in a mammal.

53. (canceled)

54. A method of enhancing phagocytosis by a macrophage, the method comprising contacting the macrophage with the compound of claim 32.

55. The method of claim 54, wherein at least one of the following applies:
(a) the contacting inhibits at least in part the biological activity of a signal regulatory protein alpha (SIRPα) expressed on the surface of the macrophage;
(b) the contacting takes place in the vicinity of the cell to be phagocytized by the macrophage;
(c) the cell is at least partially coated by opsonizing antibodies;
(d) the cell to be phagocytized is cancerous;
(e) the method is performed in vivo in a subject suffering from cancer, optionally wherein the compound is administered systemically to the subject or wherein the compound is administered to the vicinity of the cancer and/or intratumorally in the subject, optionally wherein the subject is human.

56-62. (canceled)


SEQUENCE LISTING (19 sequences)

SEQ ID NO: 1 - protein, 7 aa, Artificial Sequence
  Peptide
  misc_feature (4)..(4): Xaa can be any naturally occurring amino acid
VTQXRQG

SEQ ID NO: 2 - protein, 8 aa, Artificial Sequence
  nS-wt
VTELTREG

SEQ ID NO: 3 - protein, 8 aa, Artificial Sequence
  nS-F
VTELFREG

SEQ ID NO: 4 - protein, 8 aa, Artificial Sequence
  nS-V
VTELVREG

SEQ ID NO: 5 - protein, 8 aa, Artificial Sequence
  nS-X
LETVEGTR

SEQ ID NO: 6 - protein, 9 aa, Artificial Sequence
  nS-Cyc
  MISC_FEATURE (1)..(1): Binding site for cysteine 9
  MISC_FEATURE (9)..(9): Binding site for cysteine 1
CVELTREGC

SEQ ID NO: 7 - protein, 10 aa, Artificial Sequence
  nS-F-Cyc
  MISC_FEATURE (1)..(1): Binding site for cysteine 10
  MISC_FEATURE (10)..(10): Binding site for cysteine 1
CVTELFREGC

SEQ ID NO: 8 - protein, 10 aa, Artificial Sequence
  nS-X-Cyc
  MISC_FEATURE (1)..(1): Binding site for cysteine 10
  MISC_FEATURE (10)..(10): Binding site for cysteine 1
CLETVEGTRC

SEQ ID NO: 9 - protein, 17 aa, Artificial Sequence
  nS-FF
  MISC_FEATURE (8)..(9): 5x PEG linker between residues
  MISC_FEATURE (9)..(10): 5x PEG linker between residues
VTELFREGKGERFLETV

SEQ ID NO: 10 - protein, 17 aa, Artificial Sequence
  nS-VV
  MISC_FEATURE (8)..(9): 5x PEG linker between residues
  MISC_FEATURE (9)..(10): 5x PEG linker between residues
VTELVREGKGERVLETV

SEQ ID NO: 11 - protein, 17 aa, Artificial Sequence
  nS-XX
  MISC_FEATURE (8)..(9): 5x PEG linker between residues
  MISC_FEATURE (9)..(10): 5x PEG linker between residues
LETVEGTRKRTGEVTEL

SEQ ID NO: 12 - protein, 19 aa, Artificial Sequence
  nS-F4
  MISC_FEATURE (1)..(8): Two VTELFREG-PEGx5 groups attached to lysine 9
  MISC_FEATURE (8)..(9): 5x PEG linker between residues
  MISC_FEATURE (11)..(12): 5x PEG linker between residues
  MISC_FEATURE (12)..(19): Two VTELFREG-PEGx5 groups attached to lysine 11
VTELFREGKKKGERFLETV

SEQ ID NO: 13 - protein, 114 aa, Artificial Sequence
  HumanV2
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVR

SEQ ID NO: 14 - protein, 113 aa, Artificial Sequence
  NOD
TEVKVIQPEKSVSVAAGDSTVLNCTLTSLLPVGPIRWYRGVGQSRQLIYSFTTEHFPRVTNVSDATKRSNLDFSIRISNVTPEDAGTYYCVKFQRGSPDTEIQSGGGTEVYVL

SEQ ID NO: 15 - protein, 116 aa, Artificial Sequence
  BALB/c
GTEVKVTQPEKSVSVAAGDSTILNCTVTSLLPVGPIRWYRGVGQSRLLIYSFTGEHFPRIRNVSDTTKRNNMDFSIRISNVTPEDAGTYYCVKFQRGSSEPDTEIQSGGGTEVYVL

SEQ ID NO: 16 - protein, 116 aa, Artificial Sequence
  C57
GKELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEVYVL

SEQ ID NO: 17 - protein, 10 aa, Artificial Sequence
  XP_027364751.1
CVPELTREGC

SEQ ID NO: 18 - protein, 10 aa, Artificial Sequence
  EF117015.1
CVTELFREVS

SEQ ID NO: 19 - protein, 10 aa, Artificial Sequence
  MBA2374999.1
CLETVDGTRS